SEQUENCE LISTING (121 sequences)

SEQ ID NO: 1 - protein, 254 aa, Homo sapiens
  REGION 1..254: note = misc_feature - human 4-1BBL, full length, Uniprot P41273
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 205 aa, Homo sapiens
  REGION 1..205: note = misc_feature - human 4-1BBL, ectodomain, aa 50-254
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 3 - protein, 203 aa, Homo sapiens
  REGION 1..203: note = misc_feature - human 4-1BBL, aa 52-254
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 4 - protein, 197 aa, Homo sapiens
  REGION 1..197: note = misc_feature - human 4-1BBL, aa 52-248
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 5 - protein, 184 aa, Homo sapiens
  REGION 1..184: note = misc_feature - human 4-1BBL, aa 71-254
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 6 - protein, 178 aa, Homo sapiens
  REGION 1..178: note = misc_feature - human, 4-1BBL, aa 71-248
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 7 - protein, 175 aa, Homo sapiens
  REGION 1..175: note = misc_feature - human, 4-1BBL, aa 80-254
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 8 - protein, 169 aa, Homo sapiens
  REGION 1..169: note = misc_feature - human, 4-1BBL, aa 80-248
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 9 - protein, 170 aa, Homo sapiens
  REGION 1..170: note = misc_feature - human, 4-1BBL, aa 85-254
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 10 - protein, 164 aa, Homo sapiens
  REGION 1..164: note = misc_feature - human, 4-1BBL, aa 85-248
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 11 - protein, 183 aa, Homo sapiens
  REGION 1..183: note = misc_feature - human OX40L (CD252), full length
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 12 - protein, 133 aa, Homo sapiens
  REGION 1..133: note = misc_feature - human OX40L (CD252), ectodomain, aa 51-183
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 13 - protein, 132 aa, Homo sapiens
  REGION 1..132: note = misc_feature - human OX40L (CD252), aa 52-183
VSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 14 - protein, 261 aa, Homo sapiens
  REGION 1..261: note = misc_feature - human CD40L, full length
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 15 - protein, 215 aa, Homo sapiens
  REGION 1..215: note = misc_feature - human, CD40L, ectodomain, aa 47-261
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 16 - protein, 124 aa, Homo sapiens
  REGION 1..124: note = misc_feature - human CD40L, aa 138-261
LQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 17 - protein, 199 aa, Homo sapiens
  REGION 1..199: note = misc_feature - human GITRL, full length
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 18 - protein, 128 aa, Homo sapiens
  REGION 1..128: note = misc_feature - human GITRL, ectodomain, 72-199
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 19 - protein, 193 aa, Homo sapiens
  REGION 1..193: note = misc_feature - human CD70, full length
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 20 - protein, 155 aa, Homo sapiens
  REGION 1..155: note = misc_feature - human CD70, ectodomain, aa 39-193
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 21 - protein, 240 aa, Homo sapiens
  REGION 1..240: note = misc_feature - human LIGHT, from Merrimack, full length
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 22 - protein, 182 aa, Homo sapiens
  REGION 1..182: note = misc_feature - human LIGHT, from Merrimack, ectodomain, aa 59-240
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 23 - protein, 167 aa, Homo sapiens
  REGION 1..167: note = misc_feature - human LIGHT, from Merrimack, ectodomain, aa 74-240
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 24 - protein, 234 aa, Homo sapiens
  REGION 1..234: note = misc_feature - human CD30L, full length
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 25 - protein, 172 aa, Homo sapiens
  REGION 1..172: note = misc_feature - human CD30L, ectodomain, aa 63-234
QRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 26 - protein, 281 aa, Homo sapiens [sequence omitted]

SEQ ID NO: 27 - protein, 243 aa, Homo sapiens
  REGION 1..243: note = misc_feature - human TRAIL, ectodomain, aa 39-281
TNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 28 - protein, 251 aa, Homo sapiens
  REGION 1..251: note = misc_feature - human TL1A, full length
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 29 - protein, 159 aa, Homo sapiens
  REGION 1..159: note = misc_feature - human TL1A, ectodomain, aa 93-251
DKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 30 - protein, 302 aa, Homo sapiens
  REGION 1..302: note = misc_feature - human ICOSL, from Uniprot O75144, full length
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 31 - protein, 238 aa, Homo sapiens [sequence omitted]

SEQ ID NO: 32 - protein, 288 aa, Homo sapiens
  REGION 1..288: note = misc_feature - human CD80, from Uniprot P33681, full length
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 33 - protein, 208 aa, Homo sapiens [sequence omitted]

SEQ ID NO: 34 - protein, 329 aa, Homo sapiens
  REGION 1..329: note = misc_feature - human CD86, from Uniprot P42081, full length
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 35 - protein, 224 aa, Homo sapiens
  REGION 1..224: note = misc_feature - human CD86, ectodomain, aa 24-247
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 36 - protein, 378 aa, Homo sapiens
  REGION 1..378: note = misc_feature - human TIM-4, from Uniprot Q96H15-1, full length
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 37 - protein, 335 aa, Homo sapiens
  REGION 1..335: note = misc_feature - human SLAMF1, from Uniprot Q13291, full length
MDPKGLLSLTFVLFLSLAFGASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 38 - protein, 217 aa, Homo sapiens
  REGION 1..217: note = misc_feature - human SLAMF1, ectodomain, aa 21-237
ASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKP

SEQ ID NO: 39 - protein, 250 aa, Homo sapiens
  REGION 1..250: note = misc_feature - human CD58, from Uniprot P19256, full length
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 40 - protein, 92 aa, Homo sapiens
  REGION 1..92: note = misc_feature - human CD58, ectodomain, aa 29-120
FSQQIYGVVYGNVTFHVPSNVPLKEVLWKKQKDKVAELENSEFRAFSSFKNRVYLDTVSGSLTIYNLTSSDEDEYEMESPNITDTMKFFLYV

SEQ ID NO: 41 - protein, 332 aa, Homo sapiens
  REGION 1..332: note = misc_feature - human SLAMF6, from Uniprot Q96DU3, full length
MLWLFQSLLFVFCFGPGNVVSQSSLTPLMVNGILGESVTLPLEFPAGEKVNFITWLFNETSLAFIVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKMILFMVSGICIVFGFIILLLLVLRKRRDSLSLSTQRTQGPAESARNLEYVSVSPTNNTVYASVTHSNRETEIWTPRENDTITIYSTINHSKESKPTFSRATALDNVV

SEQ ID NO: 42 - protein, 204 aa, Homo sapiens
  REGION 1..204: note = misc_feature - human SLAMF6, ectodomain, aa 22-225
QSSLTPLMVNGILGESVTLPLEFPAGEKVNFITWLFNETSLAFIVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTK

SEQ ID NO: 43 - protein, 285 aa, Homo sapiens
  REGION 1..285: note = misc_feature - human SLAMF8, from Uniprot Q9P0V8
MVMRPLWSLLLWEALLPITVTGAQVLSKVGGSVLLVAARPPGFQVREAIWRSLWPSEELLATFFRGSLETLYHSRFLGRAQLHSNLSLELGPLESGDSGNFSVLMVDTRGQPWTQTLQLKVYDAVPRPVVQVFIAVERDAQPSKTCQVFLSCWAPNISEITYSWRRETTMDFGMEPHSLFTDGQVLSISLGPGDRDVAYSCIVSNPVSWDLATVTPWDSCHHEAAPGKASYKDVLLVVVPVSLLLMLVTLFSAWHWCPCSGKKKKDVHADRVGPETENPLVQDLP

SEQ ID NO: 44 - protein, 212 aa, Homo sapiens
  REGION 1..212: note = misc_feature - human SLAMF8, ectodomain, aa 23-234
AQVLSKVGGSVLLVAARPPGFQVREAIWRSLWPSEELLATFFRGSLETLYHSRFLGRAQLHSNLSLELGPLESGDSGNFSVLMVDTRGQPWTQTLQLKVYDAVPRPVVQVFIAVERDAQPSKTCQVFLSCWAPNISEITYSWRRETTMDFGMEPHSLFTDGQVLSISLGPGDRDVAYSCIVSNPVSWDLATVTPWDSCHHEAAPGKASYKDV

SEQ ID NO: 45 - protein, 8 aa, synthetic construct
  REGION 1..8: note = Synthetic: AB987 (M5) CDR1
GFTFSTYS

SEQ ID NO: 46 - protein, 8 aa, synthetic construct
  REGION 1..8: note = Synthetic: AB988 (M23) CDR1
GYTFTSYD

SEQ ID NO: 47 - protein, 10 aa, synthetic construct
  REGION 1..10: note = Synthetic: AB989 (M11) CDR1
GFSLSTNVMC

SEQ ID NO: 48 - protein, 8 aa, synthetic construct
  REGION 1..8: note = Synthetic: AB990 CDR1
GFTFSNCA

SEQ ID NO: 49 - protein, 8 aa, synthetic construct
  REGION 1..8: note = Synthetic: AB991 (M14) CDR1
RFTFSNYW

SEQ ID NO: 50 - protein, 8 aa, synthetic construct
  REGION 1..8: note = Synthetic: AB987 (M5) CDR2
ISGSSGHI

SEQ ID NO: 51 - protein, 8 aa, synthetic construct
  REGION 1..8: note = Synthetic: AB988 (M23) CDR2
MNPNSGNT

SEQ ID NO: 52 - protein, 7 aa, synthetic construct
  REGION 1..7: note = Synthetic: AB989 (M11) CDR2
IDWDDDK

SEQ ID NO: 53 - protein, 8 aa, synthetic construct
  REGION 1..8: note = Synthetic: AB990 CDR2
ISGSGGGT

SEQ ID NO: 54 - protein, 8 aa, synthetic construct
  REGION 1..8: note = Synthetic: AB991 (M14) CDR2
IKQDGSEK

SEQ ID NO: 55 - protein, 11 aa, synthetic construct
  REGION 1..11: note = Synthetic: AB987 (M5) CDR3
ASMHYYYYLDV

SEQ ID NO: 56 - protein, 12 aa, synthetic construct
  REGION 1..12: note = Synthetic: AB988 (M23) CDR3
AITGTTAGTFDI

SEQ ID NO: 57 - protein, 17 aa, synthetic construct
  REGION 1..17: note = Synthetic: AB989 (M11) CDR3
ARTGITGTHYYYYYMDV

SEQ ID NO: 58 - protein, 12 aa, synthetic construct
  REGION 1..12: note = Synthetic: AB990 CDR3
AKANWNYAYFDF

SEQ ID NO: 59 - protein, 18 aa, synthetic construct
  REGION 1..18: note = Synthetic: AB991 (M14) CDR3
AREGGYCSDTRCFFYMDV

SEQ ID NO: 60 - protein, 9 aa, synthetic construct
  REGION 1..9: note = Synthetic: AB987 CDR1
TGTVTTSNY

SEQ ID NO: 61 - protein, 6 aa, synthetic construct
  REGION 1..6: note = Synthetic: AB988 CDR1
QGISSY

SEQ ID NO: 62 - protein, 6 aa, synthetic construct
  REGION 1..6: note = Synthetic: AB989 CDR1
QGIRND

SEQ ID NO: 63 - protein, 6 aa, synthetic construct
  REGION 1..6: note = Synthetic: AB990 CDR1
QRVSSN

SEQ ID NO: 64 - protein, 6 aa, synthetic construct
  REGION 1..6: note = Synthetic: AB991 CDR1
QSIISW

SEQ ID NO: 65-69 - skipped (5 entries)

SEQ ID NO: 70 - protein, 9 aa, synthetic construct
  REGION 1..9: note = Synthetic: AB987 CDR3
ALWYSNHWV

SEQ ID NO: 71 - protein, 9 aa, synthetic construct
  REGION 1..9: note = Synthetic: AB988 CDR3
QQHNSYPWT

SEQ ID NO: 72 - protein, 9 aa, synthetic construct
  REGION 1..9: note = Synthetic: AB989 CDR3
LQDYNYPWT

SEQ ID NO: 73 - protein, 10 aa, synthetic construct
  REGION 1..10: note = Synthetic: AB990 CDR3
QQYDIWPMYT

SEQ ID NO: 74 - protein, 9 aa, synthetic construct
  REGION 1..9: note = Synthetic: AB991 CDR3
QQYNSYSRT

SEQ ID NO: 75 - protein, 118 aa, synthetic construct
  REGION 1..118: note = Synthetic: AB987
EVQLVESGGGLVTPGGSLRLSCAASGFTFSTYSMNWVRQAPGKGLEWVSSISGSSGHIYYADSVRGRFTISRDNAKNSLYLQMNSLRAEDTAVYYCASMHYYYYLDVWGKGTTVTVSS

SEQ ID NO: 76 - protein, 109 aa, synthetic construct
  REGION 1..109: note = Synthetic: AB987
QAVVTQESALTTSPGETVTLTCRSSTGTVTTSNYVTWVQEKPDHLFTGLIGATNNRAPGVPARFSGSLIGDKAALTITGAQTEDEAIYFCALWYSNHWVFGGGTKLTVL

SEQ ID NO: 77 - protein, 126 aa, synthetic construct
  REGION 1..126: note = Synthetic: AB988
QVQPVQSGAEVKKPGASVKVSCKASGYTFTSYDINWVRQATGQGLEWMGWMNPNSGNTGYAQKFQGRVTMTRNTSISTAYMELSSLRSEDTAVYYCAITGTTAGTFDIWGQGTMVTVSQDISMISS

SEQ ID NO: 78 - protein, 107 aa, synthetic construct
  REGION 1..107: note = Synthetic: AB988
DIQLTQSPSFLSASVGARVTTTCRASQGISSYLAWYEQKPGKAPKLLIYAASTLQSGVPSRFSGSGSGTEFTLTISSLQPEDSATYYCQQHNSYPWTFGQGTKVEIK

SEQ ID NO: 79 - protein, 125 aa, synthetic construct
  REGION 1..125: note = Synthetic: AB989
QVTLRESGPALVKPTQTLTLTCTFSGFSLSTNVMCVSWIRQPPGKALEWLALIDWDDDKYYSTSLKTRLTISKDTSKNQVVLTMTNMDPVDTATYYCARTGITGTHYYYYYMDVWGKGTTVTVSS

SEQ ID NO: 80 - protein, 107 aa, synthetic construct
  REGION 1..107: note = Synthetic: AB989
AIQMTQSPSSLSASVGDRVTITCRASQGIRNDLGWYQQKPGKAPKLLIYAASSLQSGVPSRFSGSGFGTDFTLTISSLQPEDFATYYCLQDYNYPWTFGQGTKVEIK

SEQ ID NO: 81 - protein, 119 aa, synthetic construct
  REGION 1..119: note = Synthetic: AB990
EVQLVESGGGLVQPGGSLRLSCAASGFTFSNCAMNWVRQAPGKGLEWVSVISGSGGGTYYADSVKGRFTISRDNSKNTLYLQMNSLRAEDTAVYYCAKANWNYAYFDFWGQGTLVTVSS

SEQ ID NO: 82 - protein, 108 aa, synthetic construct
  REGION 1..108: note = Synthetic: AB990
EIVMTQSPATLSVSPGERATLSCRASQRVSSNLAWYQQKPGQAPRLLIYGASTRATGIPARFSGSGSGTEFTLTINSLQSADFAVFFCQQYDIWPMYTFGQGTKLEIK

SEQ ID NO: 83 - protein, 125 aa, synthetic construct
  REGION 1..125: note = Synthetic: AB991
EVQVVESGGGLVQPGGSLRLSCAASRFTFSNYWMSWVRQAPGRGLEWVANIKQDGSEKYYVDSVKGRFTISRDNAKNSLYLQMNSLRAEDTAVYYCAREGGYCSDTRCFFYMDVWGKGTTVTVSS

SEQ ID NO: 84 - protein, 107 aa, synthetic construct
  REGION 1..107: note = Synthetic: AB991
DIQMTQSPSTLSASVGDRVTITCRASQSIISWLAWYQQKPGKAPKLLIYKASSLEGGVPSRFSGSGSGTDFTLTISSLQPDDFATYYCQQYNSYSRTFGQGTTVEIK

SEQ ID NO: 85 - protein, 243 aa, Homo sapiens
  REGION 1..243: note = misc_feature - human Collagen triple helix repeat-containing protein 1 Isoform 1 Gene (CTHRC1), Uniprot Accession number Q96CG8-1
MRPQGPAASPQRLRGLLLLLLLQLPAPSSASEIPKGKQKAQLRQREVVDLYNGMCLQGPAGVPGRDGSPGANGIPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPEMNSTINIHRTSSVEGLCEGIGAGLVDVAIWVGTCSDYPKGDASTGWNSVSRIIIEELPK

SEQ ID NO: 86 - protein, 232 aa, Homo sapiens
  REGION 1..232: note = misc_feature - human Collagen triple helix repeat-containing protein 1 Isoform 2 Gene (CTHRC1), Uniprot Accession number Q96CG8-2
MWPPGRSITVKLREKTVSRKLEMNGPSAFQGLICGKYNGMCLQGPAGVPGRDGSPGANGIPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPEMNSTINIHRTSSVEGLCEGIGAGLVDVAIWVGTCSDYPKGDASTGWNSVSRIIIEELPIYML

SEQ ID NO: 87 - protein, 229 aa, Homo sapiens
  REGION 1..229: note = misc_feature - human Collagen triple helix repeat-containing protein 1 Isoform 3 Gene (CTHRC1) Uniprot Accession number Q96CG8-3
MWPPGRSITVKLREKTVSRKLEMNGPSAFQGLICGKYNGMCLQGPAGVPGRDGSPGANGIPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPEMNSTINIHRTSSVEGLCEGIGAGLVDVAIWVGTCSDYPKGDASTGWNSVSRIIIEELPK

SEQ ID NO: 88 - protein, 354 aa, synthetic construct
  REGION 1..354: note = Synthetic: AB987 heavy chain polynucleotide
GAGGTGCAGTTGGTAGAAAGCGGCGGTGGACTCGTGACCCCTGGTGGGTCTCTTCGCTTATCTTGCGCTGCTTCCGGCTTCACTTTTTCTACGTACAGTATGAATTGGGTTCGACAGGCACCCGGCAAGGGCCTGGAATGGGTGTCCAGTATCTCGGGATCCTCAGGACATATTTACTACGCTGATAGCGTGAGGGGGCGGTTCACAATCAGCCGTGATAATGCCAAGAACTCTCTCTATCTACAAATGAACAGTCTGAGAGCAGAGGACACAGCCGTCTACTACTGTGCCTCAATGCACTACTATTATTATCTGGACGTCTGGGGGAAAGGTACTACCGTTACCGTGAGCTCC

SEQ ID NO: 89 - protein, 327 aa, synthetic construct
  REGION 1..327: note = Synthetic: AB987 light chain polynucleotide
CAGGCAGTGGTGACTCAGGAATCCGCTCTTACAACTTCCCCCGGCGAAACCGTAACATTAACCTGCAGATCAAGTACCGGAACCGTGACTACATCTAACTATGTCACCTGGGTCCAAGAGAAGCCTGATCACCTGTTCACCGGCTTGATTGGAGCCACAAATAACCGGGCCCCGGGTGTTCCAGCGAGGTTCAGCGGATCTCTCATCGGTGACAAAGCTGCCCTCACAATCACCGGGGCTCAGACTGAGGACGAGGCAATATACTTTTGTGCCCTGTGGTACAGCAATCATTGGGTTTTTGGTGGGGGCACTAAGCTGACGGTGCTA

SEQ ID NO: 90 - protein, 378 aa, synthetic construct
  REGION 1..378: note = Synthetic: AB988 heavy chain polynucleotide
CAGGTCCAGCCTGTGCAATCAGGAGCCGAAGTTAAGAAGCCCGGGGCTAGCGTTAAAGTGAGTTGTAAAGCTTCCGGTTACACTTTTACATCTTATGATATCAACTGGGTGCGCCAGGCGACTGGCCAGGGACTCGAGTGGATGGGCTGGATGAATCCAAATTCCGGTAACACCGGCTACGCCCAAAAGTTCCAGGGACGGGTGACCATGACCAGGAACACATCCATTTCGACCGCATACATGGAGCTGAGCAGTTTGAGATCCGAAGATACCGCTGTATATTATTGCGCAATTACAGGCACCACTGCCGGGACGTTCGACATCTGGGGGCAGGGTACTATGGTGACAGTCTCTCAGGACATATCAATGATCAGCTCT

SEQ ID NO: 91 - protein, 321 aa, synthetic construct
  REGION 1..321: note = Synthetic: AB988 light chain polynucleotide
GACATCCAGCTCACTCAATCGCCTTCCTTTCTGTCAGCTTCTGTCGGAGCCAGGGTGACTACCACCTGTCGGGCTAGTCAGGGAATAAGCTCTTACTTGGCATGGTACGAGCAGAAACCAGGGAAGGCACCAAAGCTACTGATCTATGCGGCCTCCACTCTGCAGTCAGGTGTGCCTAGCAGATTCTCTGGCAGCGGGTCTGGCACGGAGTTTACACTTACAATTTCCAGCCTCCAACCCGAAGATAGTGCCACATACTATTGCCAGCAGCACAACTCCTATCCCTGGACCTTCGGCCAGGGTACCAAGGTTGAAATTAAA

SEQ ID NO: 92 - protein, 375 aa, synthetic construct
  REGION 1..375: note = Synthetic: AB989 heavy chain polynucleotide
CAGGTGACATTGAGAGAATCCGGCCCCGCACTGGTCAAGCCCACCCAGACTCTAACATTAACCTGCACTTTTTCTGGGTTCAGTCTGTCGACCAACGTGATGTGCGTCTCATGGATAAGGCAACCTCCAGGTAAAGCTCTGGAGTGGCTCGCCCTTATTGACTGGGATGATGACAAATATTACAGCACCAGCCTGAAGACGCGGCTCACCATCTCAAAGGACACTAGTAAAAACCAGGTGGTTTTGACGATGACCAATATGGATCCTGTAGACACAGCCACTTACTATTGTGCTCGCACAGGAATCACAGGGACTCACTACTACTATTACTATATGGATGTGTGGGGCAAGGGAACAACCGTGACCGTTTCTTCC

SEQ ID NO: 93 - protein, 321 aa, synthetic construct
  REGION 1..321: note = Synthetic: AB989 light chain polynucleotide
GCCATCCAGATGACACAAAGCCCATCCAGTCTGTCTGCCAGCGTTGGAGACCGGGTGACTATAACCTGTAGAGCATCACAGGGCATCCGCAACGATCTGGGGTGGTATCAGCAGAAGCCAGGTAAAGCACCCAAGTTGCTCATTTATGCCGCTTCGTCTCTTCAGTCTGGAGTCCCTTCCAGGTTTTCCGGCAGTGGTTTCGGCACAGACTTTACTCTAACCATTTCAAGCCTGCAACCCGAAGATTTCGCTACCTACTACTGCCTCCAGGACTATAATTACCCTTGGACGTTCGGACAGGGGACAAAGGTGGAGATCAAA

SEQ ID NO: 94 - protein, 357 aa, synthetic construct
  REGION 1..357: note = Synthetic: AB990 heavy chain polynucleotide
GAGGTGCAGCTGGTTGAATCTGGCGGAGGGCTGGTCCAACCCGGAGGCTCCCTGCGCCTCAGTTGCGCTGCATCCGGCTTTACTTTCTCTAACTGTGCTATGAACTGGGTGAGACAGGCCCCTGGTAAAGGTCTCGAATGGGTGAGCGTGATTAGCGGCAGCGGCGGAGGTACATATTATGCCGATTCCGTAAAAGGGCGATTCACCATCTCACGGGACAATTCAAAGAATACCTTGTACCTTCAGATGAACTCTCTAAGGGCAGAGGACACAGCCGTGTACTATTGCGCCAAGGCTAATTGGAACTACGCGTACTTTGATTTCTGGGGACAGGGGACTTTAGTCACCGTTAGTTCG

SEQ ID NO: 95 - protein, 324 aa, synthetic construct
  REGION 1..324: note = Synthetic: AB990 light chain polynucleotide
GAAATCGTGATGACTCAGTCTCCAGCCACTCTCTCTGTTTCACCCGGAGAGCGTGCTACACTGTCCTGCAGAGCATCCCAACGAGTGTCCAGCAATCTAGCCTGGTACCAGCAGAAGCCAGGGCAGGCGCCTCGCCTGCTTATCTATGGGGCTTCTACACGGGCTACCGGTATTCCCGCAAGGTTCAGCGGCTCAGGAAGTGGCACCGAGTTTACACTGACCATAAACAGTTTGCAGAGCGCCGACTTTGCCGTCTTCTTTTGTCAGCAGTATGATATCTGGCCTATGTACACGTTCGGCCAAGGTACCAAACTCGAAATTAAG

SEQ ID NO: 96 - protein, 375 aa, synthetic construct
  REGION 1..375: note = Synthetic: AB991 heavy chain polynucleotide
GAGGTGCAGGTTGTCGAATCGGGAGGCGGGCTGGTGCAGCCCGGTGGGAGTCTGAGACTCTCCTGCGCTGCATCAAGGTTCACCTTTAGCAATTACTGGATGTCCTGGGTGCGTCAAGCTCCTGGTAGGGGGCTGGAATGGGTCGCCAATATTAAGCAGGATGGTAGCGAGAAGTACTATGTCGACTCTGTAAAAGGCCGGTTTACAATCTCCCGAGACAACGCCAAAAACAGTCTTTATCTACAGATGAACAGCTTGCGCGCAGAGGACACTGCCGTGTACTACTGTGCGCGGGAGGGAGGCTATTGCTCTGATACCAGATGTTTCTTCTATATGGATGTGTGGGGAAAGGGCACCACTGTGACAGTTTCTTCA

SEQ ID NO: 97 - protein, 321 aa, synthetic construct
  REGION 1..321: note = Synthetic: AB991 light chain polynucleotide
GACATTCAAATGACTCAATCTCCATCCACTCTATCCGCATCAGTGGGCGATCGGGTGACCATCACATGTAGAGCTTCCCAGAGCATTATATCTTGGTTGGCGTGGTACCAGCAGAAACCTGGCAAGGCTCCCAAGCTTCTGATCTATAAGGCCTCATCTCTCGAAGGTGGAGTCCCCAGCCGCTTTAGCGGGTCCGGGAGTGGTACCGACTTTACGCTGACAATCAGTTCGCTGCAGCCTGATGACTTCGCCACCTATTACTGCCAGCAGTACAACAGCTATTCTAGGACCTTCGGACAGGGCACAACTGTTGAGATCAAA

SEQ ID NO: 98 - protein, 5 aa, synthetic construct
  REGION 1..5: note = Synthetic: Linker
GGGGS

SEQ ID NO: 99 - skipped

SEQ ID NO: 100 - protein, 15 aa, synthetic construct
  REGION 1..15: note = Synthetic: Linker, ours
GGGGSGGGGSGGGGS

SEQ ID NO: 101 - protein, 440 aa, synthetic construct
  REGION 1..440: note = Synthetic: M23-41BBL
QVQPVQSGAEVKKPGASVKVSCKASGYTFTSYDINWVRQATGQGLEWMGWMNPNSGNTGYAQKFQGRVTMTRNTSISTAYMELSSLRSEDTAVYYCAITGTTAGTFDIWGQGTMVTVSSGGGGSGGGGSGGGGSDIQLTQSPSFLSASVGARVTTTCRASQGISSYLAWYEQKPGKAPKLLIYAASTLQSGVPSRFSGSGSGTEFTLTISSLQPEDSATYYCQQHNSYPWTFGQGTKVEIKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 102 - protein, 241 aa, synthetic construct
  REGION 1..241: note = Synthetic: M23 scFv
QVQPVQSGAEVKKPGASVKVSCKASGYTFTSYDINWVRQATGQGLEWMGWMNPNSGNTGYAQKFQGRVTMTRNTSISTAYMELSSLRSEDTAVYYCAITGTTAGTFDIWGQGTMVTVSSGGGGSGGGGSGGGGSDIQLTQSPSFLSASVGARVTTTCRASQGISSYLAWYEQKPGKAPKLLIYAASTLQSGVPSRFSGSGSGTEFTLTISSLQPEDSATYYCQQHNSYPWTFGQGTKVEIK

SEQ ID NO: 103 - protein, 440 aa, synthetic construct
  REGION 1..440: note = Synthetic: M23-41BBL, alternate, VH and VL reversed
DIQLTQSPSFLSASVGARVTTTCRASQGISSYLAWYEQKPGKAPKLLIYAASTLQSGVPSRFSGSGSGTEFTLTISSLQPEDSATYYCQQHNSYPWTFGQGTKVEIKGGGGSGGGGSGGGGSQVQPVQSGAEVKKPGASVKVSCKASGYTFTSYDINWVRQATGQGLEWMGWMNPNSGNTGYAQKFQGRVTMTRNTSISTAYMELSSLRSEDTAVYYCAITGTTAGTFDIWGQGTMVTVSSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 104 - protein, 241 aa, synthetic construct
  REGION 1..241: note = Synthetic: M23 alternate scFv
DIQLTQSPSFLSASVGARVTTTCRASQGISSYLAWYEQKPGKAPKLLIYAASTLQSGVPSRFSGSGSGTEFTLTISSLQPEDSATYYCQQHNSYPWTFGQGTKVEIKGGGGSGGGGSGGGGSQVQPVQSGAEVKKPGASVKVSCKASGYTFTSYDINWVRQATGQGLEWMGWMNPNSGNTGYAQKFQGRVTMTRNTSISTAYMELSSLRSEDTAVYYCAITGTTAGTFDIWGQGTMVTVSS

SEQ ID NO: 105 - protein, 441 aa, synthetic construct
  REGION 1..441: note = Synthetic: M5-41BBL
EVQLVESGGGLVTPGGSLRLSCAASGFTFSTYSMNWVRQAPGKGLEWVSSISGSSGHIYYADSVRGRFTISRDNAKNSLYLQMNSLRAEDTAVYYCASMHYYYYLDVWGKGTTVTVSSGGGGSGGGGSGGGGSQAVVTQESALTTSPGETVTLTCRSSTGTVTTSNYVTWVQEKPDHLFTGLIGATNNRAPGVPARFSGSLIGDKAALTITGAQTEDEAIYFCALWYSNHWVFGGGTKLTVLGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 106 - protein, 242 aa, synthetic construct
  REGION 1..242: note = Synthetic: M5 scFv
EVQLVESGGGLVTPGGSLRLSCAASGFTFSTYSMNWVRQAPGKGLEWVSSISGSSGHIYYADSVRGRFTISRDNAKNSLYLQMNSLRAEDTAVYYCASMHYYYYLDVWGKGTTVTVSSGGGGSGGGGSGGGGSQAVVTQESALTTSPGETVTLTCRSSTGTVTTSNYVTWVQEKPDHLFTGLIGATNNRAPGVPARFSGSLIGDKAALTITGAQTEDEAIYFCALWYSNHWVFGGGTKLTVL

SEQ ID NO: 107 - protein, 441 aa, synthetic construct
  REGION 1..441: note = Synthetic: M5-41BBL alternate construct
QAVVTQESALTTSPGETVTLTCRSSTGTVTTSNYVTWVQEKPDHLFTGLIGATNNRAPGVPARFSGSLIGDKAALTITGAQTEDEAIYFCALWYSNHWVFGGGTKLTVLGGGGSGGGGSGGGGSEVQLVESGGGLVTPGGSLRLSCAASGFTFSTYSMNWVRQAPGKGLEWVSSISGSSGHIYYADSVRGRFTISRDNAKNSLYLQMNSLRAEDTAVYYCASMHYYYYLDVWGKGTTVTVSSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 108 - protein, 242 aa, synthetic construct
  REGION 1..242: note = Synthetic: M5 alternate scFv
QAVVTQESALTTSPGETVTLTCRSSTGTVTTSNYVTWVQEKPDHLFTGLIGATNNRAPGVPARFSGSLIGDKAALTITGAQTEDEAIYFCALWYSNHWVFGGGTKLTVLGGGGSGGGGSGGGGSEVQLVESGGGLVTPGGSLRLSCAASGFTFSTYSMNWVRQAPGKGLEWVSSISGSSGHIYYADSVRGRFTISRDNAKNSLYLQMNSLRAEDTAVYYCASMHYYYYLDVWGKGTTVTVSS

SEQ ID NO: 109 - protein, 10 aa, synthetic construct
  REGION 1..10: note = Synthetic: linker
GGGGSGGGGS

SEQ ID NO: 110 - protein, 10 aa, synthetic construct
  REGION 1..10: note = Synthetic: linker
SGGGGSGGGG

SEQ ID NO: 111 - protein, 14 aa, synthetic construct
  REGION 1..14: note = Synthetic: linker
GGGGSGGGGSGGGG

SEQ ID NO: 112 - protein, 20 aa, synthetic construct
  REGION 1..20: note = Synthetic: linker
GGGGSGGGGSGGGGSGGGGS

SEQ ID NO: 113 - protein, 10 aa, synthetic construct
  REGION 1..10: note = Synthetic: linker
GSPGSSSSGS

SEQ ID NO: 114 - protein, 8 aa, synthetic construct
  REGION 1..8: note = Synthetic: linker
GSGSGSGS

SEQ ID NO: 115 - protein, 8 aa, synthetic construct
  REGION 1..8: note = Synthetic: linker
GSGSGNGS

SEQ ID NO: 116 - protein, 8 aa, synthetic construct
  REGION 1..8: note = Synthetic: linker
GGSGSGSG

SEQ ID NO: 117 - protein, 6 aa, synthetic construct
  REGION 1..6: note = Synthetic: linker
GGSGSG

SEQ ID NO: 118 - protein, 4 aa, synthetic construct
  REGION 1..4: note = Synthetic: linker
GGSG

SEQ ID NO: 119 - protein, 8 aa, synthetic construct
  REGION 1..8: note = Synthetic: linker
GGSGNGSG

SEQ ID NO: 120 - protein, 8 aa, synthetic construct
  REGION 1..8: note = Synthetic: linker
GGNGSGSG

SEQ ID NO: 121 - protein, 6 aa, synthetic construct
  REGION 1..6: note = Synthetic: linker
GGNGSG

(19) United States
(12) Patent Application Publication
(10) Pub. No.: US 2025/0215070 A1
(43) Pub. Date: Jul. 3, 2025

(54) ANTI-CTHRC1 FUSION PROTEINS AND METHODS OF USING THE SAME

(71) Applicant: PHENOMIC AI, Toronto (CA)

(72) Inventors: Sam Cooper, Toronto (CA); Christopher Harvey, Boston, MA (US); Michael Briskin, Boston, MA (US)

(21) Appl. No.: 18/728,058

(22) PCT Filed: Jan. 10, 2023
(86) PCT No.: PCT/US2023/010528

§ 371 Date: Jul. 10, 2024

Related U.S. Application Data
(60) Provisional application No. 63/298,194, filed on Jan. 10, 2022.

(51) Int. Cl. C07K 16/18; A61K 39/00; A61P 35/00; C07K 14/705

(52) U.S. Cl. CPC C07K 16/18; A61P 35/00; C07K 14/70578; A61K 2039/505; C07K 2317/622; C07K 2317/732; C07K 2319/00

(57) ABSTRACT

The present invention is directed to anti-CTHRC1 fusion proteins, compositions comprising the same, and methods of using such anti-CTHRC1 fusion proteins and compositions for the prevention and/or treatment of cancer. Wherein anti-CTHRC1 fusion proteins comprise a first domain comprising a CTHRC1 binding moiety; and a second domain comprising a T cell engager; preferably wherein the T cell engager comprises a ligand for a receptor expressed on a T cell.

### CROSS-REFERENCE TO RELATED APPLICATIONS

This application claims the benefit of U.S. Provisional Application No. 63/298,194, filed Jan. 10, 2022, which is incorporated herein by reference in its entirety.

### BACKGROUND

Lymphocyte activation typically requires the convergence of two signals. In T cells the first emerges from activation of the antigen specific T-cell receptors caused by engagement of TCR with MHC receptors displaying neoantigen; in the context of cancer these are mutated proteins. (Allison, McIntyre and Block, J. Immunology 129(5):2293-2300 (1982)). Following initial T cell receptor ligation and activation or priming, lymphocytes upregulate a series of surface receptors deemed costimulatory molecules. These surface proteins bind ligands on accessory cells to further enhance and sustain T cell function. CD28 was the first T-cell costimulatory molecule to be discovered and remains the prototypical immune activator. (June et al., Molecular and Cellular Biology 7(12):4472-81 (1987)). CD28 is an activating receptor and engages B7.1 and B7.2 ligands on antigen presenting cells (APC's). (Hatchock, et al., J. Exp. Med. https://doi.org/10.1084/jem.180.2.631 (1994)). This signal combined with T-cell receptor activation via neoantigen loaded MHC-class 2 receptors on the APC then triggers differentiation of the naive T-cell into proliferating effector populations, and release of pro-inflammatory cytokines that potentiate the response.

A conceptually similar series of events occurs to drive NK cell activation. In NK-cells, MHC class-1 receptors inhibit activation of NK-cell receptors independently of antigen. Thus, cells expressing low MHC class-1 trigger the first activation signal in NK-cells. (Ljunggren and Karre, Immunology Today 11(7): 237-44 (1990)). Alternatively, the first signal may come from activation of the NKG2D receptors through the MHC-like molecules, MICA/B and ULBP1-6 protein classes. (Schmiedel and Mandelboim, Frontiers in Immunology 9 (September):2040 (2018)). The second signal emerges from the convergence of signaling from NK-cell co-stimulatory receptors that can be either inhibitory or activating, for example KIRS or NKP30/NKP46 respectively. (Chen and Flies, Nature Reviews. Immunology 13(4):227-42 (2013)). Collectively, with both signals an anti-tumor immune response is triggered resulting in immune-mediated clearance of the tumor, though sufficient signal from T/NK-cell costimulatory receptors alone can trigger response. (Chen and Flies (2013)).

Costimulatory pathways are also conserved across cell types, including, for example, expression of the IgG receptor FcGRIIIA where engagement leads to a strong inflammatory response. (Bryceson et al., Immunological Reviews 214 (December): 73-91 (2006)). Commonly viewed as a receptor on NK cells, this receptor is also expressed on macrophages, B cells, and CD8 T cells. (Mellor, et al., Journal of Hematology & Oncology 6 (January): 1 (2013)). Additional examples of costimulatory receptors with wide cell specificity are the TNF family receptors such as GITR and 4-1-BB. These receptors induce activation of both T-cells and NK-cells. (Barao, Frontiers in Immunology 3: 402 (2012)). While the TNF family receptor CD27 is expressed by NK-cells, T-cells and B-cells. (Buchan, Rogel, and Al-Shamkhani, Blood 131 (1): 39-48 (2018)). Delivery of costimulatory agents is therefore capable of affecting activity of a variety of cells of the immune system.

To prevent over-activation, innate feedback mechanisms trigger the up-regulation of inhibitory T-cell costimulatory receptors including PD1 and CTLA4 that suppress the co-stimulatory signals and reduce the cell-killing capacity of the T-cell population (Chen and Flies (2013)), but may also facilitate the evasion of transformed tumor cells to immune-mediated destruction. Tumor cells also shed ligands to NK cell receptors, which have been suggested to down-regulate costimulatory molecules and reduce cytotoxic function. Accordingly, while the mutations leading to tumorigenesis have the potential to induce neoantigens which are capable of triggering immune responses, tumors as a whole have been shown to downregulate costimulatory molecule ligand expression at either the transcript or protein level to avoid immune surveillance. The prototypical T cell costimulatory receptor CD28 has largely been shown to be the target of CTLA-4 as well as PD-1 mediated T cell exhaustion, either by direct competition for its ligands CD80/86 by CTLA-4 or via PD-1 mediated suppression of signal transduction to limit activation. (Hui, et al., Science 355 (6332): 1428-33 (2017)). Signaling via other T cell costimulatory molecules, particularly 4-1BB and ICOS has been suggested to remain intact even in the context of PD-1-mediated exhaustion. (Habib-Agahi, Jaberipour, and Searle, Cellular Immunology 256 (1-2): 39-46 (2009)).

Providing additional costimulation has long been viewed as a promising therapeutic strategy to treat cancer as well as a number of other indications. Across costimulatory molecules, 4-1BB activation has been suggested as a viable therapeutic strategy as 4-1BBL treatment has been shown to restore CD28 expression in in vitro systems. (Habib-Agahi, Jaberipour, and Searle (2009)). Agonist antibodies to 4-1BB as well as other TNFa family members (OX40, GITR, CD40) have been developed and tested preclinically, with several entering clinical development. Additional preclinical and clinical development programs have targeted the costimulatory molecules ICOS, CD27, and CD28. Unfortunately, however, preclinical success has not translated clinically for these antibodies, which may be attributable to a narrow window for maximizing therapeutic efficacy while limiting systemic toxicity associated with global immune activation. A range of promising clinical studies have failed, many of which were due to treatment-associated AEs occurring. For example, in a study of healthy volunteers being treated with a super-agonist to the B7.1 and 2 receptor CD28 demonstrated severe toxicity, with cytokine release syndrome being seen in all patients administered this therapy. (Hünig, The FEBS Journal 283 (18): 3325-34 (2016)). Agonists to OX40, CD40, and 4-1BB have also been met with high incidence of treatment-related adverse events, which negatively impacted their clinical development. (Segal, et al., Clinical Cancer Research: An Official Journal of the American Association for Cancer Research 23 (8): 1929-36 (2017)). CD40 agonists, for example, observed high rates of AEs in Phase 1 safety studies at a number of sub-efficacious doses. (Calvo, et al., Journal of Clinical Orthodontics: JCO 37 (15_suppl): 2527-2527 (2019)).

Collectively, this indicates that the natural window for lymphocyte specific activation in the tumor may be higher than that to induce systemic effects of costimulatory molecule activation, suggesting targeted delivery may be a necessary route forward to deliver accumulation of drug and stimulation at tumor sites. Tethering costimulatory molecules via tumor antigens may also provide added cross-linking necessary to drive costimulatory activity that may be limited when relying on Fc interactions in the context of an agonist mAb.

Tumor stroma represents a primary barrier to anti-tumor immunity and therapeutic targeting, with stroma largely considered a major immunosuppressive component that facilitates resistance to immune checkpoint inhibitors. Tumor targeted ligands offer an innovative solution to relieving stromal mediated immune-suppression and may provide several advantages. For example, while cancer cells can often quickly evolve to down/up-regulate many specific targets, fibroblast/macrophage genetic information is stable. As a result, the highly selective stromal target Fibroblast Activating Protein has been developed as a means of targeting T-cell and NK cell co-stimulatory receptor agonists to the tumor microenvironment.

Yet, readouts from FAP targeted IO therapy studies to date have been mixed, with simlukafusp alfa (Fap-targeted IL-2) failing to show clear tumor regressions as a single agent or provide much additive activity to anti-PD-L1 alone. (“ASCO 2021: Randomized Phase Ib Study To Evaluate Safety, Pharmacokinetics and Therapeutic Activity of Simlukafusp α in Combination With Atezolizumab±Bevacizumab in Patients With Unresectable Advanced/Metastatic Renal Cell Carcinoma (Rcc) (NCT03063762)” n.d.). Future studies exploring alternative combination treatments may improve results, but limitations with the activity of FAP blockade alone, coupled to the fact that FAP targeted agents likely only bind to CAFs, indicate that new inventions are required to effectively relieve stroma mediated immune suppression, and simultaneously target cancer cells. Successful relief of stromal mediated immune suppression in concert with the activation of immune cells to directly attack cancer cells offers an opportunity to address challenges in targeting solid tumors, and thus improve outcomes for patients with these diseases. The present invention addresses this and other unmet needs.

### SUMMARY

As demonstrated herein for the first time, antibodies developed against the stromal target CTHRC1 display activity in reprogramming the tumor microenvironment as well as combination efficacy with PD1 inhibitors. CTHRC1 mRNA expression is also highly selective to the tumor, with expression on both CAFs, and to a lesser extent by cancer cells. Given the selective localization of CTHRC1 to the tumor, the use of CTHRC1-based agents for targeting costimulatory ligands to the tumor microenvironment provides a unique and effective solution for the foregoing challenges in the prior art.

To that end, the present disclosure provides anti-CTHRC1 fusion proteins and compositions thereof, where the anti-CTHRC1 fusion protein comprises a collagen triple helix repeat containing 1 (CTHRC) binding moiety and a T cell engager, preferably wherein the T cell engager comprises a ligand for a receptor expressed on a T cell. In embodiments, the T cell engager comprises a ligand for a costimulatory receptor, or a functional fragment, variant or mutant thereof. In embodiments, the costimulatory receptor is a tumor necrosis factor superfamily (TNFSF) member, or an Immunoglobulin superfamily (IgSF) member. In embodiments, the costimulatory receptor is selected from the group consisting of 4-1BB (CD137), OX40 (CD134), CD40, CD27, CD28, herpesvirus entry mediator (HVEM), ICOS, CD2, and GITR The present disclosure further provides methods for making the same and the use of the same in the treatment of cancer.

In one aspect, an anti-collagen triple helix repeat containing 1 (anti-CTHRC1) fusion protein, comprises: (a) a first domain comprising a CTHRC1 binding moiety; and (b) a second domain comprising a T cell engager; preferably wherein the T cell engager comprises a ligand for a receptor expressed on a T cell.

In embodiments, the T cell engager comprises a ligand for a costimulatory receptor expressed on a T cell, or a functional fragment, variant or mutant thereof, preferably wherein the costimulatory receptor is a member of the tumor necrosis factor superfamily (TNFSF).

In embodiments, the costimulatory receptor is selected from the group consisting of 4-1BB (CD137), OX40 (CD134), CD40, CD27, CD28, herpesvirus entry mediator (HVEM), ICOS, CD2, and GITR.

In embodiments, where the costimulatory receptor is 4-1BB (CD137); preferably wherein the ligand comprises an amino acid sequence having at least 80% identity to any one of SEQ ID NOs: 1-10, or a functional fragment thereof.

In embodiments, where the costimulatory receptor is OX40 (CD134); preferably wherein the ligand comprises an amino acid sequence having at least 80% identity to any one of SEQ ID NOs: 11-13, or a functional fragment thereof.

In embodiments, where the costimulatory receptor is CD40; preferably wherein the ligand comprises an amino acid sequence having at least 80% identity to any one of SEQ ID NOs: 14-16, or a functional fragment thereof.

In embodiments, where the costimulatory receptor is GITR; preferably wherein the ligand comprises an amino acid sequence having at least 80% identity to any one of SEQ ID NOs: 17-18 or a functional fragment thereof.

In embodiments, where the costimulatory receptor is CD27; preferably wherein the ligand comprises an amino acid sequence having at least 80% identity to any one of SEQ ID NOs: 19-20, or a functional fragment thereof.

In embodiments, where the costimulatory receptor is HVEM; preferably wherein the ligand comprises an amino acid sequence having at least 80% identity to any one of SEQ ID NOs: 21-23, or a functional fragment thereof.

In embodiments, where the costimulatory receptor is ICOS; preferably wherein the ligand comprises an amino acid sequence having at least 80% identity to any one of SEQ ID NOs: 30-31, or a functional fragment thereof.

In embodiments, where the costimulatory receptor is CD28; preferably wherein the ligand comprises an amino acid sequence having at least 80% identity to any one of SEQ ID NOs: 32-35, or a functional fragment thereof.

In embodiments, where the costimulatory receptor is CD2; preferably wherein the ligand comprises an amino acid sequence having at least 80% identity to any one of SEQ ID NOs: 39-40, or a functional fragment thereof.

In embodiments, the anti-CTHRC1 fusion protein further comprises a linker positioned between the CTHRC1 binding moiety and the ligand; preferably wherein the linker is selected from the group consisting of (G₄S)ₙ or (GSG)ₙ, where n is 1 to 4.

In embodiments, the anti-CTHRC1 binding moiety comprises an anti-CTHRC1 antibody which binds to CTHRC1; preferably wherein the CTHRC1 antibody binds to CTHRC1 with a binding affinity of less than 10 nM.

In embodiments, the anti-CTHRC1 antibody comprises a heavy chain variable region comprising a CDR1 sequence comprising the sequence of SEQ ID NO: 46; a CDR2 sequence comprising the sequence of SEQ ID NO: 51; and a CDR3 sequence comprising the sequence of SEQ ID NO: 56; and a light chain variable region comprising a CDR1 sequence comprising the sequence of SEQ ID NO: 61; a CDR2 sequence comprising the sequence of SEQ ID NO: 66; and a CDR3 sequence comprising the sequence of SEQ ID NO: 71.

In embodiments, the anti-CTHRC1 antibody comprises a heavy chain variable region comprising SEQ ID NO: 77 and a light chain variable region comprising SEQ ID NO: 78.

In embodiments, the anti-CTHRC1 antibody comprises a heavy chain variable region comprising a CDR1 sequence comprising the sequence of SEQ ID NO: 45; a CDR2 sequence comprising the sequence of SEQ ID NO: 50; and a CDR3 sequence comprising the sequence of SEQ ID NO: 55; and a light chain variable region comprising a CDR1 sequence comprising the sequence of SEQ ID NO: 60; a CDR2 sequence comprising the sequence of SEQ ID NO: 65; and a CDR3 sequence comprising the sequence of SEQ ID NO: 70.

In embodiments, the anti-CTHRC1 antibody comprises a heavy chain variable region comprising SEQ ID NO: 75 and a light chain variable region comprising SEQ ID NO: 76.

In embodiments, the anti-CTHRC1 antibody comprises a heavy chain variable region comprising an amino acid sequence selected from the group consisting of: SEQ ID NOs: 75, 77, 79, 81, and 83.

In embodiments, the anti-CTHRC1 antibody comprises a heavy chain variable region comprising an amino acid sequence selected from Table 4.

In embodiments, the anti-CTHRC1 antibody comprises a light chain variable region comprising an amino acid sequence selected from the group consisting of: SEQ ID NOs: 76, 78, 80, 82, and 84.

In embodiments, the anti-CTHRC1 antibody comprises a light chain variable region comprising an amino acid sequence selected from Table 5.

In embodiments, the anti-CTHRC1 antibody comprises a heavy chain variable region comprising a CDR1 sequence selected from the group consisting of SEQ ID NOs: 45-49; a CDR2 sequence selected from the group consisting of SEQ ID NOs: 50-54; and a CDR3 sequence selected from the group consisting of SEQ ID NOs: 55-59.

In embodiments, the anti-CTHRC1 antibody comprises a light chain variable region comprising a CDR1 sequence selected from the group consisting of SEQ ID NOs: 60-64; a CDR2 sequence selected from the group consisting of SEQ ID NOs: 65-69; and a CDR3 sequence selected from the group consisting of SEQ ID NOs: 70-74.

In embodiments, the anti-CTHRC1 antibody comprises a heavy chain variable region comprising SEQ ID NO: 75 and a light chain variable region comprising SEQ ID NO: 76.

In embodiments, the anti-CTHRC1 antibody comprises a heavy chain variable region comprising SEQ ID NO: 79 and a light chain variable region comprising SEQ ID NO: 80.

In embodiments, the anti-CTHRC1 antibody comprises a heavy chain variable region comprising SEQ ID NO: 81 and a light chain variable region comprising SEQ ID NO: 82.

In embodiments, the anti-CTHRC1 antibody comprises a heavy chain variable region comprising SEQ ID NO: 83 and a light chain variable region comprising SEQ ID NO: 84.

In embodiments, the anti-CTHRC1 antibody is a chimeric, humanized, or human antibody.

In embodiments, the anti-CTHRC1 antibody is a monoclonal antibody.

In embodiments, the anti-CTHRC1 antibody is an antibody fragment.

In embodiments, the anti-CTHRC1 antibody comprises a single-chain antibody.

In embodiments, the anti-CTHRC1 antibody is a heavy-chain only antibody (single domain antibody).

In embodiments, a method for activating T cells in a tumor microenvironment comprises contacting a tumor with the anti-CTHRC1 fusion protein or any of the foregoing embodiments.

In embodiments, a method of inhibiting the growth of a cell displaying a CTHRC1 tumor epitope comprises contacting said cell with the anti-CTHRC1 fusion protein of any of the foregoing embodiments.

In embodiments, a method of treating a subject having cancer comprises contacting the cell with the anti-CTHRC1 fusion protein of any of the foregoing embodiments.

In embodiments, the cancer is selected from the group consisting of breast cancer, lung cancer, colon cancer, ovarian cancer, melanoma cancer, bladder cancer, renal cancer, kidney cancer, liver cancer, head and neck cancer, colorectal cancer, melanoma, pancreatic cancer, gastric carcinoma cancer, esophageal cancer, mesothelioma, prostate cancer, leukemia, lymphomas, and myelomas.

In embodiments, the foregoing methods can further comprise administering an allogenic or autologous T cell therapy in combination with the anti-CTHRC1 fusion protein of any of the foregoing embodiments.

In embodiments, a pharmaceutical composition comprises the anti-CTHRC1 fusion protein of any of the foregoing embodiments and a pharmaceutically acceptable carrier.

In embodiments, a use of the pharmaceutical composition of the foregoing embodiment can be in the preparation of a medicament for the treatment of a cell proliferative disorder, preferably cancer.

### BRIEF DESCRIPTION OF THE DRAWINGS

FIG. 1A depicts a sample from a colorectal cancer patient stained with FAP showing tumor and stroma.

FIG. 1B depicts a sample from a colorectal cancer patient stained with anti-CTHRC1 antibody showing tumor and stroma.

FIG. 2A depicts FACS data for staining of cancer cell lines with anti-CTHRC1 antibodies.

FIG. 2B depicts FACS data for staining of cancer cell lines with anti-CTHRC1 antibodies.

FIG. 3A depicts degree of proliferation for Jurkat T cells stained with CFSE. 2-way ANOVA *p<0.5 ****p<0.001. All h41BBL vs M23-h41BBL NS.

FIG. 3B depicts flow cytometry data for Jurkat T cells.

FIG. 4 depicts tumor volume over time for mice treated with control (Dengue-41BBL) or an anti-CTHRC1 fusion protein (M23-41BBL).

FIG. 5 depicts the survival curve for mice treated with control (Dengue-41BBL), an anti-CTHRC1 fusion protein (M23-41BBL) or an anti-CTHRC1 fusion protein (M5-41BBL (low dose)).

FIG. 6 depicts tumor volume over time for mice treated with control (Dengue-41BBL), an anti-CTHRC1 fusion protein (M23-41BBL) or an anti-CTHRC1 fusion protein (M5-41BBL).

FIG. 7 depicts tumor volume over time for mice treated with control (Isotype Control) or an anti-CTHRC1 fusion protein (M23-41BBL) at varying doses.

FIGS. 8A-8B illustrate that CTHRC1 mRNA is a top ranked marker of cancer-associated fibroblasts (CAFs) in cancer-rich, immune-cold, tumor samples. Aggregated tumor samples were profiled across cancer scRNA studies, to create a large scRNA atlas. Samples were then grouped into T-cell rich, cancer poor samples (immune-hot), and cancer-rich, T-cell poor samples (immune-cold) (FIG. 8A). Genes expressed by CAFs were compared between the two groups, to determine which associate most with cancer rich samples (Wilcoxon Rank). These genes were then filtered down to those specifically expressed by CAFs in all samples (top 500; Wilcoxon rank). CTHRC1 was the 1₁ₕ highest ranked gene in the analysis (FIG. 8B).

FIG. 9 illustrates that CTHRC1 is highly upregulated in cancer vs. normal adjacent tissues, as determined in bulk RNA measurements analyzed from The Cancer Genome Atlas. Across the set of indications profiled, the highest levels of CTHRC1 expression was seen in solid cancers, notably breast, lung, ovarian, pancreatic, sarcoma, melanoma, and uterine carcinosarcomas (P<0.001 in all cases tested). Collectively this indicates that CTHRC1 expression is highest in more desmoplastic, stromal rich cancers, in line with the notion that it is principally a CAF secreted target.

FIG. 10 depicts a series of survival plots showing CTHRC1 survival curves for a number of solid tumors where CTHRC1 is associated with poor survival. Values were derived from The Cancer Genome Atlas. An online tool, GEPIA, was used to calculate and plot the survival curves.

FIG. 11 illustrates that CTHRC1 levels increase with stage in liver cancer (left) and colorectal cancer (right) alongside other indications (not shown). In both cases, p<0.05; Student's T test between Stage I and Stage IV. Stage II and Stage III display intermediate CTHRC1 expression levels.

FIGS. 12A-12C are histograms showing bulk-RNA expression level of known stromal targets FAP (FIG. 12A) and LRRC15 (FIG. 12B) alongside CTHRC1 (FIG. 12C) in pancreatic cancer samples (The Cancer Genome Atlas) vs. all normal tissue samples (GTEX). The histograms highlight that a significant therapeutic window exists for targeting CTHRC1 in pancreatic cancer, based on bulk-RNA measurements. This window is similar, if not larger, than that of the known/developed stromal targets FAP and LRRC15.

FIG. 13 is a dataset illustrating that very high levels of CTHRC1 expression is seen in CAFs across many solid cancers, as well as cancer epithelial expression in breast, pancreatic, lung, ovarian, and skin cancers. In contrast, minimal CTHRC1 expression is seen in normal tissue. The data was obtained based on previously generated large integrated single-cell RNA-sequence Atlas to enable probing of gene-expression at the single cell level across 11 ontoxics cancer and normal tissue samples (Swechha, 2021). The data highlights a potentially large therapeutic window for blocking CTHRC1 and value in using mAbs against CTHRC1 for targeting payloads, e.g., ADCs, to the tumor microenvironment.

FIG. 14 is a dataset using the same atlas discussed with regard to FIG. 10, performed for LRRC15, a known non-toxic stromal target, where antibodies ADCs have been engineered and shown to be safe in the clinic. Low levels of LRRC15 were seen in normal tissue as compared to CTHRC1. In cancerous single-cell RNA (scRNA) datasets it was observed that LRRC15 expression, unlike CTHRC1, is more selectively localized to specific CAFs in specific breast cancers, and low levels of expression are also seen on sarcoma cancer cells.

FIG. 15 is a graph illustrating quantitative ELISA of CTHRC1 in mono-culture and co-culture. Supernatants from a set of different mono-cultures (fibroblast or cancer cells) and co-cultures (fibroblasts and cancer cells) were profiled for CTHRC1 levels. CTHRC1 is expressed at low levels in fibroblast mono-cultures (BJ, CCD18-Co) and upregulated in co-culture, indicating that interactions between fibroblasts and cancer cells drives CTHRC1 expression.

FIG. 16 shows a series of images of tissue from three mouse models probed with CTHRC1 specific mAbs. Broad staining was observed within tumor regions, indicating CTHRC1 protein localizes to cancer regions in vivo.

FIG. 17 shows a series of images of tissue from three human cancers, showing variation in CTHRC1 expression patterns at the protein level, indicative of different expression dynamics. In Head and Neck and Melanoma cancer samples, CTHRC1 localizes to interfaces between cancer and stromal tissue. In pancreatic cancer, CTHRC1 is seen broadly expressed on regions dense in CAFs/stromal tissue.

FIGS. 18A-18C illustrate efficacy of CTHRC1 tested in the syngeneic mouse breast tumor model, EMT6. Results are shown for three different anti-CTHRC1 antibodies, specifically M5 (FIG. 18A), M14 (FIG. 18B), and M14 (FIG. 18C).

FIGS. 19A-19B illustrate efficacy of anti-CTHRC1 (clone M5) in the PD-1 resistant Pan02 pancreatic cancer model.

FIG. 20 illustrates the effect of preconditioning of cells with anti-CTHRC1 antibody on infiltration of CD8 T cells.

### DETAILED DESCRIPTION

The present disclosure provides anti-CTHRC1 fusion proteins and compositions thereof, where the anti-CTHRC1 fusion protein comprises a collagen triple helix repeat containing 1 (CTHRC) binding moiety and a T cell engager, e.g., a ligand for a receptor expressed on a T cell. In embodiments, the T cell engager comprises a ligand for a costimulatory receptor, or a functional fragment, variant or mutant thereof. In embodiments, the T cell engager comprises a cytokine, e.g. IL-2, or a functional fragment, variant or mutant thereof. The present disclosure further provides methods for making the same and the use of the same in the treatment of cancer.

The practice of the present invention will employ, unless otherwise indicated, conventional techniques of molecular biology (including recombinant techniques), microbiology, cell biology, biochemistry, and immunology, which are within the skill of the art.

Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### Definitions

For purposes of interpreting this specification, the following definitions will apply and whenever appropriate, terms used in the singular will also include the plural and vice versa. In the event that any definition set forth conflicts with any document incorporated herein by reference, the definition set forth below shall control.

As used herein, the singular forms of “a,” “an,” and “the” include plural referents unless the context clearly dictates otherwise.

The use of the term “or” in the claims and the present disclosure is intended to mean “and/or” unless explicitly indicated to refer to alternatives only or the alternatives are mutually exclusive.

Use of the term “about,” when used with a numerical value, is intended to include +/−10%. By way of example, but not limitation, if a number of amino acids is identified as “about 100,” this would include 90 to 110 (plus or minus 10%).

As used herein “comprising,” “including,” “containing,” “having” and the like are intended to be open-ended and inclusive such that they do not exclude additional, unrecited elements unless otherwise noted.

The term “Collagen Triple Helix Repeat Containing 1 (CTHRC1)”, as used herein, refers to any native CTHRC1 from any vertebrate source, including mammals such as primates (e.g., humans, primates, and rodents (e.g., mice and rats), unless otherwise indicated. The term encompasses several isoforms (see, e.g., SEQ ID NOs: 85-87). Human CTHRC1 is encoded by the nucleotide sequence corresponding to GenBank Accession No. NG031985.

The term “Collagen Triple Helix Repeat Containing 1” encompasses “full-length,” unprocessed CTHRC1 as well as any form of CTHRC1 that results from processing in the cell. The term encompasses naturally occurring variants of CTHRC1, e.g., splice variants, allelic variants and isoforms. The CTHRC1 polypeptides described herein may be isolated from a variety of sources, such as from human tissue types or from another source, or prepared by recombinant or synthetic methods. A “native sequence CTHRC1 polypeptide” comprises a polypeptide having the same amino acid sequence as the corresponding CTHRC1 polypeptide derived from nature. Such native sequence CTHRC1 polypeptides can be isolated from nature or can be produced by recombinant or synthetic means. The term “native sequence CTHRC1 polypeptide” specifically encompasses naturally-occurring truncated or secreted forms of the specific CTHRC1 polypeptide (e.g., an extracellular domain sequence), naturally-occurring variant forms (e.g., alternatively spliced forms) and naturally-occurring allelic variants of the polypeptide. In certain embodiments of the invention, the native sequence CTHRC1 polypeptides disclosed herein are mature or full-length native sequence polypeptides comprising the full-length amino acid sequences shown in the accompanying disclosure.

As used herein, “costimulatory receptor” refers to the cognate binding partner on an immune cell that specifically binds with a costimulatory ligand, thereby mediating a costimulatory response by the cell, such as, but not limited to, proliferation. Costimulatory receptors are cell surface molecules other than antigen receptors or their ligands that are required for an efficient immune response. Costimulatory receptors include, but are not limited to, an MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1 Id, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD48, STING, and a ligand that specifically binds with CD83.

As used herein, “ligand” refers to an agent that binds to a natural receptor under normal physiological conditions (e.g. 4-1BBL).

A “fragment” is a portion of a polypeptide that includes a region that contains at least 20%, 30%, 40%, 50%, 60%, 70%, 80%, 90%, 95%, 98%, 99% or any range therebetween of a reference sequence and retains at least a portion the binding properties of the reference sequence. For example, a fragment of 4-1BB ligand (4-1BBL) can still bind to the 4-1BB receptor with at least some affinity, the same affinity, or more affinity than the original 4-1BBL sequence.

A “mutant” is a polypeptide that includes substitutions, insertions or deletions relative to a reference sequence and retains at least a portion of the binding properties of the reference sequence. For example, a mutant of 4-1BBL can still bind to 4-1BB receptor with at least some affinity, the same affinity, or more affinity than the original 4-1BBL sequence.

Percent “identity” between an amino acid sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared. In certain embodiments, default parameters are used.

A “modification” of an amino acid residue/position, as used herein, refers to a change of a primary amino acid sequence as compared to a starting amino acid sequence, wherein the change results from a sequence alteration involving said amino acid residue/positions. For example, typical modifications include substitution of the residue (or at said position) with another amino acid (e.g., a conservative or non-conservative substitution), insertion of one or more (generally fewer than 5 or 3) amino acids adjacent to said residue/position, and deletion of said residue/position. An “amino acid substitution”, or variation thereof, refers to the replacement of an existing amino acid residue in a predetermined (starting) amino acid sequence with a different amino acid residue. Generally, the modification results in alteration in at least one physicobiochemical activity of the variant polypeptide compared to a polypeptide comprising the starting (or “wild type”) amino acid sequence. For example, in the case of an antibody, a physicobiochemical activity that is altered can be binding affinity, binding capability and/or binding effect upon a target molecule.

The term “antibody” is used in the broadest sense and specifically covers, for example, single anti-CTHRC1 monoclonal antibodies (including agonist, antagonist, neutralizing antibodies, full length or intact monoclonal antibodies), anti-CTHRC1 antibody compositions with polyepitopic specificity, polyclonal antibodies, multivalent antibodies, multispecific antibodies (e.g., bispecific antibodies so long as they exhibit the desired biological activity), formed from at least two intact antibodies, single chain anti-CTHRC1 antibodies, and fragments of anti-CTHRC1 antibodies (see below), including Fab, Fab′, F(ab′)2 and Fv fragments, diabodies, single domain antibodies (sdAbs), as long as they exhibit the desired biological or immunological activity. Also included among anti-CTHRC1 antibodies, and among fragments in particular, are portions of anti-CTHRC1 antibodies (and combinations of portions of anti-CTHRC1 antibodies, for example, scFv) that may be used as targeting arms, directed to e.g., a CTHRC1 tumor epitope, in chimeric antigenic receptors of CAR-T cells, CAR-NK cells, or CAR-macrophages. Such fragments are not necessarily proteolytic fragments but rather portions of polypeptide sequences that can confer affinity for target. The term “immunoglobulin” (Ig) is used interchangeably with antibody herein. An antibody can be, for example, human, humanized and/or affinity matured.

The terms “anti-CTHRC1 antibody”, “CTHRC1 antibody”, and “an antibody that binds to CTHRC1” are used interchangeably. Anti-CTHRC1 antibodies are preferably capable of binding with sufficient affinity such that the antibody is useful as a diagnostic and/or therapeutic agent, whether in isolation or as part of fusion protein, cell, or cell composition.

In one embodiment, CTHRC1 antibody is used herein to specifically refer to an anti-CTHRC1 monoclonal antibody that (i) comprises the heavy chain variable domain of any one of SEQ ID NOs: 75, 77, 79, 81, and 83; and/or the light chain variable domain of any one of SEQ ID NOs: 76, 78, 80, 82, and 84, or (ii) comprises one, two, three, four, five, or six of the CDRs shown in Table 2 or Table 3.

An “isolated antibody” is one which has been identified and separated and/or recovered from a component of its natural environment. Contaminant components of its natural environment are materials which would interfere with therapeutic uses for the antibody, and may include enzymes, hormones, and other proteinaceous or nonproteinaceous solutes.

An “intact” antibody is one which comprises an antigen-binding site as well as a CL and at least heavy chain constant domains, CH1, CH2 and CH3. The constant domains may be native sequence constant domains (e.g., human native sequence constant domains) or amino acid sequence variant thereof. Preferably, the intact antibody has one or more effector functions.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256: 495 (1975), or may be made using recombinant DNA methods in bacterial, eukaryotic animal or plant cells (e.g., 4,816,567). The “monoclonal antibodies” may also be isolated from phage antibody libraries using the techniques described in Clackson et al., Nature, 352: 624-8 (1991) and Marks et al., J. Mol. Biol., 222: 581-97 (1991), for example.

The basic 4-chain antibody unit is a heterotetrameric glycoprotein composed of two identical light (L) chains and two identical heavy (H) chains. In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for and F isotypes.

Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, CT, 1994, at page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in CH sequence and function, e.g., humans express the following subclasses: IgG1, IgG2, IgG3, IgG4, IgA1, and IgA2.

The “variable region” or “variable domain” of an antibody refers to the amino-terminal domains of the heavy or light chain of the antibody. The variable domain of the heavy chain may be referred to as “VH” or “V_{H}” The variable domain of the light chain may be referred to as “VL” or “VL”. These domains are generally the most variable parts of an antibody and contain the antigen-binding sites.

The term “variable” refers to the fact that certain segments of the variable domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)).

“Antibody fragments” comprise a portion of an intact antibody, preferably the antigen binding or one or more variable regions of the intact antibody. Examples of antibody fragments include Fab, Fab′, F(ab′)2, and Fv fragments; diabodies; linear antibodies (see U.S. Pat. No. 5,641,870, Example 2; Zapata et al., Protein Eng. 8(10): 1057-62 (1995)); single-chain antibody molecules; and multispecific antibodies formed from antibody fragments. In one embodiment, an antibody fragment comprises an antigen binding site of the intact antibody and thus retains the ability to bind antigen. Also included among anti-CTHRC1 antibody fragments are portions of anti-CTHRC1 antibodies (and combinations of portions of anti-CTHRC1 antibodies, for example, scFv) that may be used as targeting arms, directed to e.g., a CTHRC1 tumor epitope, in chimeric antigenic receptors of CAR-T cells or CAR-NK cells, or CAR macrophages. Such fragments are not necessarily proeteolytic fragments but rather portions of polypeptide sequences that can confer affinity for target.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. In some embodiments, the sFv polypeptide further comprises a polypeptide linker between the VH and VL domains which enables the sFv to form a desired structure for antigen binding. For a review of sFv, see, e.g., Pluckthun in The Pharmacology of Monoclonal Antibodies, vol. 113, Rosenburg and Moore eds., Springer-Verlag, New York, pp. 269-315 (1994); Borrebaeck 1995, infra. In one embodiment, an anti-CTHRC1 antibody derived scFv is used as the targeting arm of a CAR-T cell, a CAR-NK cell, or a CAR-macrophage disclosed herein.

The term “hypervariable region”, “HVR”, or “HV”, when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, MD. (1991)). Chothia refers instead to the location of the structural loops (Chothia and Lesk J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures. The residues from each of these hypervariable regions are noted below.

| Loop | Kabat | AbM | Chothia | Contact |
|---|---|---|---|---|
| L1 | L24-L34 | L24-L34 | L24-L34 | L30-L36 |
| L2 | L50-L56 | L50-L56 | L50-L56 | L46-L55 |
| L3 | L89-L97 | L89-L97 | L89-L97 | L89-L96 |
| H1 | H31-H35B | H26-H35B | H26-H32 . . . 34 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H52-H56 | H47-H58 |
| H3 | H95-H102 | H95-H102 | H95-H102 | H93-H101 |

Hypervariable regions may comprise “extended hypervariable regions” as follows: 24-36 or 24-34 (L1), 46-56 or 50-56 (L2) and 89-97 (L3) in the VL and 26-35B (H1), 50-65, 47-65 or 49-65 (H2) and 93-102, 94-102 or 95-102 (H3) in the VH. The variable domain residues are numbered according to Kabat et al., supra, for each of these definitions.

“Framework” or “FR” residues are those variable domain residues other than the hypervariable region residues herein defined.

The term “variable domain residue numbering as in Kabat” or “amino acid position numbering as in Kabat”, and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g., residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g, Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system.

An antibody that “binds” an antigen or epitope of interest is one that binds the antigen or epitope with sufficient affinity that is measurably different from a non-specific interaction. Specific binding can be measured, for example, by determining binding of a molecule compared to binding of a control molecule, which generally is a molecule of similar structure that does not have binding activity.

An antibody that inhibits the growth of tumor cells is one that results in measurable growth inhibition of cancer cells. In one embodiment, an anti-CTHRC1 antibody is capable of inhibiting the growth of cancer cells displaying a CTHRC1 tumor epitope. As referred to herein, a CTHRC1 tumor epitope comprises a CTHRC1 epitope capable of being bound by an anti-CTHRC1 antibody, or fragment thereof, as herein disclosed, or capable of being at least partially bound by an antibody or other molecule that competes with an anti-CTHRC1 antibody as herein disclosed for binding to said epitope. Preferred growth inhibitory anti-CTHRC1 antibodies inhibit growth of CTHRC1-displaying tumor cells by greater than 20%, preferably from about 20% to about 50%, and even more preferably, by greater than 50% (e.g., from about 50% to about 100%) as compared to the appropriate control, the control typically being tumor cells not treated with the antibody being tested.

Anti-CTHRC1 antibodies may (i) inhibit tumor metastasis in vivo; (ii) inhibit tumor growth in vivo; (iii) decrease tumor size in vivo; (iv) inhibit tumor vascularization in vivo; (v) exhibit cycotoxic activity activity on tumor cells and cancer associated fibroblasts displaying CTHRC1 in vivo; (vi) exhibit cytostatic activity on tumor cells or cancer associated fibroblasts displaying CTHRC1 in vivo; (vii) enhance infiltration of anti-tumor immune cells in vivo or (viii) prevent suppression of immune-cells in the tumor microenvironment in vivo.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), skin cancer, melanoma, lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer (e.g., pancreatic ductal adenocarcinoma), glioblastoma, cervical cancer, ovarian cancer (e.g., high grade serous ovarian carcinoma), liver cancer (e.g., hepatocellular carcinoma (HCC)), bladder cancer (e.g., urothelial bladder cancer), testicular (germ cell tumor) cancer, hepatoma, breast cancer, brain cancer (e.g., astrocytoma), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, hepatic carcinoma, anal carcinoma, penile carcinoma, as well as head and neck cancer. Additional examples of cancer include, without limitation, adrenocortical cancer, cholangiocarcinoma, colon adenocarcinoma, B-cell lymphoma, esophageal carcinoma, glioblastoma multiforme, kidney clear cell cancer, kidney papillary cell cancer, myeloid leukemia, lung adenocarcinoma, lung squamous cancer, prostate adenocarcinoma, rectal adenocarcinoma, sarcoma, stomach adenocarcinoma, thymoma, uterine corpus, and uterine carcinosarcoma.

The term “metastatic cancer” means the state of cancer where the cancer cells of a tissue of origin are transmitted from the original site to one or more sites elsewhere in the body, by the blood vessels or lymphatics, to form one or more secondary tumors in one or more organs besides the tissue of origin. A prominent example is metastatic breast cancer.

As used herein, an “CTHRC1-associated cancer” is a cancer that is associated with over-expression of a CTHRC1 gene or gene product and/or is associated with display of a CTHRC1 epitope. Suitable control cells can be, for example, cells from an individual who is not affected with cancer or non-cancerous cells from the subject who has cancer.

Anti-CTHRC1 fusion proteins may (i) inhibit tumor metastasis in vivo; (ii) inhibit tumor growth in vivo; (iii) decrease tumor size in vivo; (iv) inhibit tumor vascularization in vivo; (v) exhibit cycotoxic activity activity on tumor cells and cancer associated fibroblasts displaying CTHRC1 in vivo; (vi) exhibit cytostatic activity on tumor cells or cancer associated fibroblasts displaying CTHRC1 in vivo; (vii) enhance infiltration of anti-tumor immune cells in vivo; or (viii) prevent suppression of immune-cells in the tumor microenvironment in vivo.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues.

The terms “patient,” “subject,” “individual,” and the like are used interchangeably herein, and refer to any animal amenable to the methods described herein. In certain non-limiting embodiments, the patient, subject or individual is a human.

The term “therapeutically effective amount”, or simply “effective amount” refers to the amount of an agent or composition (e.g., composition comprising an agent) that will elicit a biological or medical response of a tissue, system, or subject that is being sought by the researcher, veterinarian, medical doctor or other clinician. The term “therapeutically effective amount” includes that amount of an agent, or a composition comprising an agent, that, when administered, is sufficient to prevent development of, or alleviate to some extent, one or more of the signs or symptoms of the disorder or disease (e.g., hematological or solid tumor) being treated. The therapeutically effective amount will vary depending on the composition, the disease and its severity and the age, weight, etc., of the subject to be treated.

As used herein, the term “administration” means to provide or give a subject one or more agents, such as an agent that treats one or more signs or symptoms associated with a condition/disorder or disease including but not limited to cancer (e.g., lymphoma), viral infection, bacterial infection, etc., by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes. Administration “in combination with” one or more further therapeutic agents includes simultaneous (concurrent) and sequential administration in any order.

The term “pharmaceutically acceptable”, as used herein, refers to a material, including but not limited, to a salt, carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained. The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more agents, such as one or more modulatory agents. In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical agents to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

In one aspect of the invention, the anti-CTHRC1 fusion protein provides an activating signal to T-cells and NK-cells present in the tumor microenvironment.

In another aspect of the invention, the T-cell and NK-cell activating signal causes T and/or NK cell dependent cytotoxicity directed at cancer cells.

In another aspect, the anti-CTHRC1 fusion protein brings lymphocytes including T-cells and NK-cells into close proximity to CAFs or cancer cells in a CTHRC1 dependent manner.

In another aspect this enhances T and NK cell-mediated killing of CAFs and cancer cells.

In one aspect of the invention, the anti-CTHRC1 fusion protein provides a pro-inflammatory signal to the broader set of immune-cells expressing the receptor that may include, macrophages, dendritic cells, monocytes, B-cells, plasma cells, neutrophils, mast cells, and other blood derived immune cells, and antigen presenting cells.

In another aspect of the invention, the immune activating signal induces a proinflammatory immune response in the tumor microenvironment, that will result in tumor cell killing.

In another aspect of the invention the anti-CTHRC1 fusion protein brings inflammatory macrophages into close proximity to the CAF or cancer cells and induces direct macrophage mediated cell killing, for example, via complement system and complement receptor-mediated phagocytosis.

In another aspect of the invention the anti-CTHRC1 fusion protein brings inflammatory macrophages, dendritic cells or other antigen presenting cells into close proximity to CAFs and cancer cells inducing enhanced display of antigens associated with the CAF or cancer cells and associated indirect adaptive immune response.

In another aspect of the invention the proximity of immune cells triggers a broad immune response that includes release of proinflammatory cytokines.

In embodiments, an anti-CTHRC1 fusion protein is provided that can include a first domain comprising a CTHRC1 binding moiety and a second domain comprising a ligand for a receptor expressed on a T cell, preferably a human T cell. In embodiments, the second domain comprises a ligand for a costimulatory receptor. In embodiments, the second domain comprises a ligand for a cytokine receptor. In embodiments, the anti-CTHRC1 fusion protein further comprises a peptide linker positioned between the first domain and the second domain.

The presently disclosed subject matter encompasses antibody fusions. For example, proteins can be linked together either through chemical or genetic manipulation using methods known in the art. See, for example, Gillies et al., Proc. Nat'l Acad. Sci. USA 89: 1428-1432 (1992) and U.S. Pat. No. 5,650,150.

In one example, the present disclosure encompasses an anti-CTHRC1 antibody-cytokine fusion protein. In principle, an anti-CTHRC1 antibody as herein disclosed can be fused to any cytokine via the use of recombinant molecular biological techniques. In a preferred embodiment, the anti-CTHRC1 antibody is fused to IL-2 (Gillies, S., Protein Engineering, Design and Selection 26(10): 561-569 (2013); Klein, C. et al., OncoImmunology 6:3 (2017).

In another example, the present disclosure encompasses an anti-CTHRC1 antibody-T-cell engager fusion protein. Discussed herein, anti-CTHRC1 antibody-T-cell engager fusion proteins comprise fusions between an anti-CTHRC1 antibody and a ligand for a receptor expressed on a T-cell. Examples of such ligands include but are not limited to CD40L, OX40L, 4-1BBL, CD80/86, ICOSL, and the like. In embodiments, the ligand is fused to an Fc portion of an anti-CTHRC1 antibody. In embodiments, the ligand is fused to a C-terminus of a light chain of an anti-CTHRC1 antibody. Such an approach is described with regard to 4-1BBL (Dafne M. et al., Journal of Immunotherapy 38(8): 714-722 (2008), and a similar approach can be used for generation of other antibody-T-cell engager fusion proteins.

### Costimulatory Receptor Ligands

In any of the foregoing embodiments, the second domain comprises a ligand for a costimulatory receptor. In certain aspects, the costimulatory receptor is a member of the immunoglobulin superfamily (IgSF). In other aspects, the costimulatory receptor is a member of the tumor necrosis factor superfamily (TNFSF). In certain aspects, the costimulatory receptor is on an immune cell. By way of example, but not limitation, the immune cell can be a T cell, an NK cell, B cell, dendritic cell or antigen presenting cell.

In any of the foregoing embodiments, the costimulatory receptor can be selected from the group consisting of 4-1BB (CD137), OX40 (CD134), CD40, GITR, CD27, herpesvirus entry mediator (HVEM), CD30, DR3, DR5, ICOS, CD28, TIM1, SLAMF1 (CDw150), CD2, SLAMF6, SLAMF8, CD48, and STING. Preferably, the costimulatory receptor can be selected from the group consisting of 4-1BB (CD137), OX40 (CD134), CD40, CD27, CD28, herpesvirus entry mediator (HVEM), ICOS, CD2, and GITR.

In any of the foregoing embodiments, the ligand can be a natural ligand for the costimulatory receptor, a fragment thereof, a mutant thereof, or a conservatively-substituted variant of the natural ligand or fragment thereof. Alternatively, the ligand can be an engineered ligand, e.g. a non-natural ligand capable of binding to and agonizing the costimulatory receptor. Exemplary non-natural ligands are disclosed in Suo, et al., Pharmaceutics 14(1):181 (2022), Zwolak, et al., Scientific Reports 12:20538 (2022), and U.S. Patent Application Publication No. 2021/0188995, each of which is incorporated herein by reference in its entirety.

An exemplary list of costimulatory receptors and their natural ligands are provided in Table 1.

TABLE 1
Exemplary costimulatory receptors and
exemplary natural ligands thereof.

| Co-stimulatory Receptor | Exemplary Natural Ligand |
|---|---|
| 4-1BB (CD137) | 4-1BBL |
| OX40 | OX40L |
| CD40 | CD40L |
| GITR | GITRL |
| HVEM | LIGHT |
| CD28 | CD80 or CD86 |
| ICOS | ICOSL |
| TIM1 | TIM-4 |
| DR3 | TL1A |
| DR5 | TRAIL |
| SLAM | SLAM |
| CD2 | CD58 |
| SLAMF6 | SLAMF6 |
| SLAMF8 | SLAMF8 |
| CD30 | CD30L |
| CD48 | CD244 |
| STING | Cyclic GMP-AMP |

### 4-1BB Receptor

In embodiments, the costimulatory receptor can be 4-1BB (CD137). In some embodiments, the ligand can be a natural ligand of 4-1BB, e.g. 4-1BBL, a fragment thereof, a conservatively-substituted variant of the full-length or fragmentary sequence, or a mutant thereof of the full-length or fragmentary sequence. In other embodiments, the ligand can be a non-natural ligand of 4-1BB.

In embodiments, the ligand can include the sequence of SEQ ID NO: 1 or a fragment thereof, a conservatively-substituted variant of the full-length or fragmentary sequence, or a mutant thereof of the full-length or fragmentary sequence. By way of example, but not limitation, the fragment can include the sequence of any one of SEQ ID NOs: 2-10. In some embodiments, the ligand can include a sequence having at least 80%, 85%, 90%, 95%, 98%, or 99% sequence identity to any one of SEQ ID NOs: 1-10.

### OX40 Receptor

In embodiments, the costimulatory receptor can be OX40. In some embodiments, the ligand can be a natural ligand of OX40, e.g. OX40L, a fragment thereof, a conservatively-substituted variant of the full-length or fragmentary sequence, or a mutant thereof of the full-length or fragmentary sequence. In other embodiments, the ligand can be a non-natural ligand of OX40.

In embodiments, the ligand can include the sequence of SEQ ID NO: 11 or a fragment thereof, a conservatively-substituted variant of the full-length or fragmentary sequence, or a mutant thereof of the full-length or fragmentary sequence. By way of example, but not limitation, the fragment can include the sequence of any one of SEQ ID NOs: 12-13. In some embodiments, the ligand can include a sequence having at least 80%, 85%, 90%, 95%, 98%, or 99% sequence identity to any one of SEQ ID NOs: 11-13.

### CD40 Receptor

In embodiments, the costimulatory receptor can be CD40. In some embodiments, the ligand can be a natural ligand of CD40, e.g. CD40L, a fragment thereof, a conservatively-substituted variant of the full-length or fragmentary sequence, or a mutant thereof of the full-length or fragmentary sequence. In other embodiments, the ligand can be a non-natural ligand of CD40.

In embodiments, the ligand can include the sequence of SEQ ID NO: 14 or a fragment thereof, a conservatively-substituted variant of the full-length or fragmentary sequence, or a mutant thereof of the full-length or fragmentary sequence. By way of example, but not limitation, the fragment can include the sequence of any one of SEQ ID NOs: 15-16. In some embodiments, the ligand can include a sequence having at least 80%, 85%, 90%, 95%, 98%, or 99% sequence identity to any one of SEQ ID NOs: 14-16.

### GITR Receptor

In embodiments, the costimulatory receptor can be GITR. In some embodiments, the ligand a be a natural ligand of GITR, e.g. GITRL, a fragment thereof, a conservatively-substituted variant of the full-length or fragmentary sequence, or a mutant thereof of the full-length or fragmentary sequence. In other embodiments, the ligand can be a non-natural ligand of GITR.

In embodiments, the ligand can include the sequence of SEQ ID NO: 17 or a fragment thereof, a conservatively-substituted variant of the full-length or fragmentary sequence, or a mutant thereof of the full-length or fragmentary sequence. By way of example, but not limitation, the fragment can include the sequence of SEQ ID NO: 18. In some embodiments, the ligand can include a sequence having at least 80%, 85%, 90%, 95%, 98%, or 99% sequence identity to any one of SEQ ID NOs: 17-18.

### CD27 Receptor

In embodiments, the costimulatory receptor can be CD27. In some embodiments, the ligand can be a natural ligand of CD27, e.g. CD70, a fragment thereof, a conservatively-substituted variant of the full-length or fragmentary sequence, or a mutant thereof of the full-length or fragmentary sequence. In other embodiments, the ligand can be a non-natural ligand of CD27.

In embodiments, the ligand can include the sequence of SEQ ID NO: 19 or a fragment thereof, a conservatively-substituted variant of the full-length or fragmentary sequence, or a mutant thereof of the full-length or fragmentary sequence. By way of example, but not limitation, the fragment can include the sequence of SEQ ID NO: 20. In some embodiments, the ligand can include a sequence having at least 80%, 85%, 90%, 95%, 98%, or 99% sequence identity to any one of SEQ ID NOs: 19-20.

### HVEM Receptor

In embodiments, the costimulatory receptor can be HVEM. In some embodiments, the ligand a be a natural ligand of HVEM, e.g. LIGHT, a fragment thereof, a conservatively-substituted variant of the full-length or fragmentary sequence, or a mutant thereof of the full-length or fragmentary sequence. In other embodiments, the ligand can be a non-natural ligand of LIGHT.

In embodiments, the ligand can include the sequence of SEQ ID NO: 21 or a fragment thereof, a conservatively-substituted variant of the full-length or fragmentary sequence, or a mutant thereof of the full-length or fragmentary sequence. By way of example, but not limitation, the fragment can include the sequence of any one of SEQ ID NOs: 22-23. In some embodiments, the ligand can include a sequence having at least 80%, 85%, 90%, 95%, 98%, or 99% sequence identity to any one of SEQ ID NOs: 21-23.

### CD30 Receptor

In embodiments, the costimulatory receptor can be CD30. In some embodiments, the ligand can be a natural ligand of CD30, e.g. CD30L, a fragment thereof, a conservatively-substituted variant of the full-length or fragmentary sequence, or a mutant thereof of the full-length or fragmentary sequence. In other embodiments, the ligand can be a non-natural ligand of CD30.

In embodiments, the ligand can include the sequence of SEQ ID NO: 24 or a fragment thereof, a conservatively-substituted variant of the full-length or fragmentary sequence, or a mutant thereof of the full-length or fragmentary sequence. By way of example, but not limitation, the fragment can include the sequence of SEQ ID NO: 25. In some embodiments, the ligand can include a sequence having at least 80%, 85%, 90%, 95%, 98%, or 99% sequence identity to any one of SEQ ID NOs: 24-25.

### DR3 Receptor

In embodiments, the costimulatory receptor can be DR3. In some embodiments, the ligand can be a natural ligand of DR3, e.g. TRAIL, a fragment thereof, a conservatively-substituted variant of the full-length or fragmentary sequence, or a mutant thereof of the full-length or fragmentary sequence. In other embodiments, the ligand can be a non-natural ligand of DR3.

In embodiments, the ligand can include the sequence of SEQ ID NO: 26 or a fragment thereof, a conservatively-substituted variant of the full-length or fragmentary sequence, or a mutant thereof of the full-length or fragmentary sequence. By way of example, but not limitation, the fragment can include the sequence of SEQ ID NO: 27. In some embodiments, the ligand can include a sequence having at least 80%, 85%, 90%, 95%, 98%, or 99% sequence identity to any one of SEQ ID NOs: 26-27.

### DR5 Receptor

In embodiments, the costimulatory receptor can be DR5. In some embodiments, the ligand can be a natural ligand of DR5, e.g. TL1A, a fragment thereof, a conservatively-substituted variant of the full-length or fragmentary sequence, or a mutant thereof of the full-length or fragmentary sequence. In other embodiments, the ligand can be a non-natural ligand of DR5.

In embodiments, the ligand can include the sequence of SEQ ID NO: 28 or a fragment thereof, a conservatively-substituted variant of the full-length or fragmentary sequence, or a mutant thereof of the full-length or fragmentary sequence. By way of example, but not limitation, the fragment can include the sequence of SEQ ID NO: 29. In some embodiments, the ligand can include a sequence having at least 80%, 85%, 90%, 95%, 98%, or 99% sequence identity to any one of SEQ ID NOs: 28-29.

### ICOS Receptor

In embodiments, the costimulatory receptor can be ICOS. In some embodiments, the ligand can be a natural ligand of ICOS, e.g. ICOSL, a fragment thereof, a conservatively-substituted variant of the full-length or fragmentary sequence, or a mutant thereof of the full-length or fragmentary sequence. In other embodiments, the ligand can be a non-natural ligand of ICOS.

In embodiments, the ligand can include the sequence of SEQ ID NO: 30 or a fragment thereof, a conservatively-substituted variant of the full-length or fragmentary sequence, or a mutant thereof of the full-length or fragmentary sequence. By way of example, but not limitation, the fragment can include the sequence of SEQ ID NO: 31. In some embodiments, the ligand can include a sequence having at least 80%, 85%, 90%, 95%, 98%, or 99% sequence identity to any one of SEQ ID NOs: 30-31.

### CD28 Receptor

In embodiments, the costimulatory receptor can be CD28. In some embodiments, the ligand can be a natural ligand of CD28, e.g. CD80 or CD86, a fragment thereof, a conservatively-substituted variant of the full-length or fragmentary sequence, or a mutant thereof of the full-length or fragmentary sequence. In other embodiments, the ligand can be a non-natural ligand of CD28.

In embodiments, the ligand can include the sequence of SEQ ID NO: 32 or SEQ ID NO: 34 or a fragment thereof, a conservatively-substituted variant of the full-length or fragmentary sequence, or a mutant thereof of the full-length or fragmentary sequence. By way of example, but not limitation, the fragment can include the sequence of SEQ ID NO: 33 or SEQ ID NO: 35. In some embodiments, the ligand can include a sequence having at least 80%, 85%, 90%, 95%, 98%, or 99% sequence identity to any one of SEQ ID NOs: 32-35.

### TIM-1 Receptor

In embodiments, the costimulatory receptor can be TIM-1. In some embodiments, the ligand can be a natural ligand of TIM-1, e.g. TIM-4, a fragment thereof, a conservatively-substituted variant of the full-length or fragmentary sequence, or a mutant thereof of the full-length or fragmentary sequence. In other embodiments, the ligand can be a non-natural ligand of TIM-1.

In embodiments, the ligand can include the sequence of SEQ ID NO: 36 or a fragment thereof, a conservatively-substituted variant of the full-length or fragmentary sequence, or a mutant thereof of the full-length or fragmentary sequence. In some embodiments, the ligand can include a sequence having at least 80%, 85%, 90%, 95%, 98%, or 99% sequence identity to SEQ ID NO: 36 or a fragment thereof.

### SLAM Receptor

In embodiments, the costimulatory receptor can be SLAM. In some embodiments, the ligand can be a natural ligand of SLAM, e.g. SLAM, a fragment thereof, a conservatively-substituted variant of the full-length or fragmentary sequence, or a mutant thereof of the full-length or fragmentary sequence. In other embodiments, the ligand can be a non-natural ligand of SLAM.

In embodiments, the ligand can include the sequence of SEQ ID NO: 37 or a fragment thereof, a conservatively-substituted variant of the full-length or fragmentary sequence, or a mutant thereof of the full-length or fragmentary sequence. By way of example, but not limitation, the fragment can include the sequence of SEQ ID NO: 38. In some embodiments, the ligand can include a sequence having at least 80%, 85%, 90%, 95%, 98%, or 99% sequence identity to any one of SEQ ID NOs: 37-38 or a fragment thereof.

### CD2 Receptor

In embodiments, the costimulatory receptor can be CD2. In some embodiments, the ligand can be a natural ligand of CD2, e.g. CD58, a fragment thereof, a conservatively-substituted variant of the full-length or fragmentary sequence, or a mutant thereof of the full-length or fragmentary sequence. In other embodiments, the ligand can be a non-natural ligand of CD2.

In embodiments, the ligand can include the sequence of SEQ ID NO: 39 or a fragment thereof, a conservatively-substituted variant of the full-length or fragmentary sequence, or a mutant thereof of the full-length or fragmentary sequence. By way of example, but not limitation, the fragment can include the sequence of SEQ ID NO: 40. In some embodiments, the ligand can include a sequence having at least 80%, 85%, 90%, 95%, 98%, or 99% sequence identity to any one of SEQ ID NOs: 39-40.

### SLAMF6 Receptor

In embodiments, the costimulatory receptor can be SLAMF6. In some embodiments, the ligand can be a natural ligand of SLAMF6, e.g. SLAMF6, a fragment thereof, a conservatively-substituted variant of the full-length or fragmentary sequence, or a mutant thereof of the full-length or fragmentary sequence. In other embodiments, the ligand can be a non-natural ligand of SLAMF6.

In embodiments, the ligand can include the sequence of SEQ ID NO: 41 or a fragment thereof, a conservatively-substituted variant of the full-length or fragmentary sequence, or a mutant thereof of the full-length or fragmentary sequence. By way of example, but not limitation, the fragment can include the sequence of SEQ ID NO: 42. In some embodiments, the ligand can include a sequence having at least 80%, 85%, 90%, 95%, 98%, or 99% sequence identity to any one of SEQ ID NOs: 41-42 or a fragment thereof.

### SLAMF8 Receptor

In embodiments, the costimulatory receptor can be SLAMF8. In some embodiments, the ligand can be a natural ligand of SLAMF8, e.g. SLAMF8, a fragment thereof, a conservatively-substituted variant of the full-length or fragmentary sequence, or a mutant thereof of the full-length or fragmentary sequence. In other embodiments, the ligand can be a non-natural ligand of SLAMF8.

In embodiments, the ligand can include the sequence of SEQ ID NO: 43 or a fragment thereof, a conservatively-substituted variant of the full-length or fragmentary sequence, or a mutant thereof of the full-length or fragmentary sequence. By way of example, but not limitation, the fragment can include the sequence of SEQ ID NO: 44. In some embodiments, the ligand can include a sequence having at least 80%, 85%, 90%, 95%, 98%, or 99% sequence identity to any one of SEQ ID NOs: 43-44 or a fragment thereof.

### CD48

In embodiments, the costimulatory receptor can be CD48. In some embodiments, the ligand be a natural ligand of CD48, e.g. CD244, a fragment thereof, a conservatively-substituted variant of the full-length or fragmentary sequence, or a mutant thereof or of the full-length or fragmentary sequence. In other embodiments, the ligand can be a non-natural ligand of CD48.

### STING

In embodiments, the costimulatory receptor can be STING. In some embodiments, the ligand can be a natural ligand of STING, e.g. cyclic GAMP-AMP or other small molecule ligand that binds to STING. In other embodiments, the ligand can be a non-natural ligand of STING. Exemplary ligands of STING useful in the present invention are disclosed in Amouzegar, et al., Cancers 2021, 13, 2695 which is incorporated herein by reference in its entirety.

### Other Receptors

Additional costimulatory receptors, ligands (including mutants thereof) and sequences thereof are disclosed in WO 2018/144514, U.S. Patent Application Publication No. 2019/0016771, U.S. Patent Application Publication No. 2021/0169932, WO 2019/089921, WO 2017/211321, U.S. Pat. No. 9,534,056, U.S. Patent Application Publication No. 2021/0253706, U.S. Pat. No. 8,440,192, U.S. Patent Application Publication No. 2020/0758901, WO 2020/172189, WO 2017/060144, U.S. Pat. No. 11,053,293, Chen, Lieping and Flies, Dallas B., “Molecular mechanisms of T cell co-stimulation and co-inhibition,” Nat. Rev. Immunol. 13(4):227-242 (2013), Dostert, C., et al., “The TNF Family of Ligands and Receptors: Communication Modules in the Immune System and Beyond,” Physiol. Rev. 99:115-60 (2018), Li, et al., “A novel tumor-homing TRAIL variant eradicates tumor xenografts of refractory colorectal cancer cells in combination with tumor cell-targeted photodynamic therapy,” Drug Delivery 29(1):1698-1711 (2022), Meylan, et al., “TL1A and DR3, a TNF-family ligand-receptor pair that promotes lymphocyte costimulation, mycosal hyperplasia and autoimmune inflammation,” Immunol Rev. 244(1) (2011), McArdel, et al., “Roles of CD48 in regulating immunity and tolerance,” Clin. Immunol. 164:10-20 (2016), Mavaddat, et al., “Signaling Lymphocytic Activation Molecule (CDw150) is Homophilic but Self-Associates with Very Low Affinity,” J. Biol. Chem. 275(36):28100-28109 (2000), Suo, et al., Pharmaceutics 14(1):181 (2022), Zwolak, et al., Scientific Reports 12:20538 (2022), and U.S. Patent Application Publication No. 2021/0188995, each of which is incorporated by reference herein in its entirety.

It should be understood that the ligands of the present disclosure can be directed to any costimulatory receptor on any type of cell. Also contemplated herein are cognate ligands for costimulatory receptors found on NK cells, e.g. NKG2D ligands such as MICA/MICB, and ULBPs.

### CTHRC1 Binding Moiety

In embodiments of the present disclosure, the anti-CTHRC1 fusion proteins of the present disclosure include a CTHRC1 binding moiety.

In any of the foregoing embodiments, the CTHRC1 binding moiety can be an antibody, as defined herein, capable of binding to CTHRC1. In some embodiments, anti-CTHRC1 antibodies of the invention further comprise a human subgroup III heavy chain framework consensus sequence. In one embodiments of these antibodies, these antibodies further comprise a human κI light chain framework consensus sequence.

It should be understood that the CTHRC1 binding moiety can further include a peptide linker as described herein. By way of example, but not limitation, where the CTHRC1 binding moiety comprises a scFv, the VH and VL portions can be linked by any suitable linker, such as those described in the present disclosure.

Exemplary CDRs and antibody sequences are provided in the following tables:

TABLE 2
Complementarity Determining Regions, Heavy Variable Region (IMGT)

| | Heavy Chain Variable Region CDRs | | |
|---|---|---|---|
| Name | CDR1 | CDR2 | CDR3 |
| AB987 | GFTFSTYS | ISGSSGHI | ASMHYYYYLDV |
| (M5) | (SEQ ID NO: 45) | (SEQ ID NO: 50) | (SEQ ID NO: 55) |
| AB988 | GYTFTSYD | MNPNSGNT | AITGTTAGTFDI |
| (M23) | (SEQ ID NO: 46) | (SEQ ID NO: 51) | (SEQ ID NO: 56) |
| AB989 | GFSLSTNVMC | IDWDDDK | ARTGITGTHYYYYYMDV |
| (M11) | (SEQ ID NO: 47) | (SEQ ID NO: 52) | (SEQ ID NO: 57) |
| AB990 | GFTFSNCA | ISGSGGGT | AKANWNYAYFDF |
| | (SEQ ID NO: 48) | (SEQ ID NO: 53) | (SEQ ID NO: 58) |
| AB991 | RFTFSNYW | IKQDGSEK | AREGGYCSDTRCFFYMDV |
| (M14) | (SEQ ID NO: 49) | (SEQ ID NO: 54) | (SEQ ID NO: 59) |

TABLE 3
Complementarity Determining Regions, Light Variable Region (IMGT)

| | Light Chain Variable Region CDRs | | |
|---|---|---|---|
| Name | CDR1 | CDR2 | CDR3 |
| AB987 | TGTVTTSNY | ATN | ALWYSNHWV |
| | (SEQ ID NO: 60) | (SEQ ID NO: 65) | (SEQ ID NO: 70) |
| AB988 | QGISSY | AAS | QQHNSYPWT |
| | (SEQ ID NO: 61) | (SEQ ID NO: 66) | (SEQ ID NO: 71) |
| AB989 | QGIRND | AAS | LQDYNYPWT |
| | (SEQ ID NO: 62) | (SEQ ID NO: 67) | SEQ ID NO: 72) |
| AB990 | QRVSSN | GAS | QQYDIWPMYT |
| | (SEQ ID NO: 63) | SEQ ID NO: 68 | (SEQ ID NO: 73) |
| AB991 | QSIISW | KAS | QQYNSYSRT |
| | (SEQ ID NO: 64) | (SEQ ID NO: 69) | (SEQ ID NO: 74) |

TABLE 4
Illustrative Immunoglobulin Sequences-Heavy Chain Variable Region

| Name | SEQ ID | Heavy Chain Variable Region |
|---|---|---|
| AB987 | 75 | EVQLVESGGGLVTPGGSLRLSCAASGFTFSTYSMNWVRQAPGK |
| | | GLEWVSSISGSSGHIYYADSVRGRFTISRDNAKNSLYLQMNSLR |
| | | AEDTAVYYCASMHYYYYLDVWGKGTTVTVSS |
| AB988 | 77 | QVQPVQSGAEVKKPGASVKVSCKASGYTFTSYDINWVRQATG |
| | | QGLEWMGWMNPNSGNTGYAQKFQGRVTMTRNTSISTAYMEL |
| | | SSLRSEDTAVYYCAITGTTAGTFDIWGQGTMVTVSQDISMISS |
| AB989 | 79 | QVTLRESGPALVKPTQTLTLTCTFSGFSLSTNVMCVSWIRQPPG |
| | | KALEWLALIDWDDDKYYSTSLKTRLTISKDTSKNQVVLTMTN |
| | | MDPVDTATYYCARTGITGTHYYYYYMDVWGKGTTVTVSS |
| AB990 | 81 | EVQLVESGGGLVQPGGSLRLSCAASGFTFSNCAMNWVRQAPG |
| | | KGLEWVSVISGSGGGTYYADSVKGRFTISRDNSKNTLYLQMNS |
| | | LRAEDTAVYYCAKANWNYAYFDFWGQGTLVTVSS |
| AB991 | 83 | EVQVVESGGGLVQPGGSLRLSCAASRFTFSNYWMSWVRQAPG |
| | | RGLEWVANIKQDGSEKYYVDSVKGRFTISRDNAKNSLYLQMN |
| | | SLRAEDTAVYYCAREGGYCSDTRCFFYMDVWGKGTTVTVSS |

TABLE 5
Illustrative Immunoglobulin Sequences-Light Chain Variable Region

| Name | SEQ ID | Light Chain Variable Region |
|---|---|---|
| AB987 | 76 | QAVVTQESALTTSPGETVTLTCRSSTGTVTTSNYVTWVQEKPD |
| | | HLFTGLIGATNNRAPGVPARFSGSLIGDKAALTITGAQTEDEAIY |
| | | FCALWYSNHWVFGGGTKLTVL |
| AB988 | 78 | DIQLTQSPSFLSASVGARVTTTCRASQGISSYLAWYEQKPGKAP |
| | | KLLIYAASTLQSGVPSRFSGSGSGTEFTLTISSLQPEDSATYYCQ |
| | | QHNSYPWTFGQGTKVEIK |
| AB989 | 80 | AIQMTQSPSSLSASVGDRVTITCRASQGIRNDLGWYQQKPGKA |
| | | PKLLIYAASSLQSGVPSRFSGSGFGTDFTLTISSLOPEDFATYYCL |
| | | QDYNYPWTFGQGTKVEIK |
| AB990 | 82 | EIVMTQSPATLSVSPGERATLSCRASQRVSSNLAWYQQKPGQA |
| | | PRLLIYGASTRATGIPARFSGSGSGTEFTLTINSLQSADFAVFFCQ |
| | | QYDIWPMYTFGQGTKLEIK |
| AB991 | 84 | DIQMTQSPSTLSASVGDRVTITCRASQSIISWLAWYQQKPGKAP |
| | | KLLIYKASSLEGGVPSRFSGSGSGTDFTLTISSLQPDDFATYYCQ |
| | | QYNSYSRTFGQGTTVEIK |

Thus, in any of the foregoing embodiments, the CTHRC1 binding moiety can comprise an antibody portion that comprises HCDR1, HCDR2, and HCDR3 of AB987, AB3988, AB3989, AB 990 or AB991 and/or LCDR1, LCDR2, and LCDR3 of A1987, AB988, AB989, AB990 or AB991 (from Tables 2 and 3). In any of the foregoing embodiments, the CTHRC1 binding moiety can comprise an antibody portion that comprises the VH and/or VL of any of AB987, AB988, AB989, AB990 or AB991 (from Tables 4 and 5).

Preferably, the CTHRC1 binding moiety of the invention is scFv, or scFab wherein the nucleic acid sequence of the scFv comprises the nucleic acid sequence(s) that encode for one or more light chain CDRs and one or more heavy chain CDRs disclosed herein for anti-CTHRC1 antibodies, and wherein the nucleic acid sequence of the scFab comprises the nucleic acid sequence(s) that encode for one or more light chain CDRs and one or more heavy chain CDRs disclosed herein for anti-CTHRC1 antibodies.

Preferably, the CTHRC1 binding moiety of the invention is an scFv, or scFab comprising an amino acid sequence selected from the group consisting of any one of SEQ ID Nos: 75-84.

Preferably, the CTHRC1 binding moiety of the invention is an scFv, or scFab comprising an amino acid sequence selected from the group consisting of any one of SEQ ID Nos: 75, 77, 79, 81, and 83, more preferably an scFv or scFab comprising an amino acid sequence selected from the group consisting of SEQ ID Nos: 77 and 83. Still more preferably, the scFv or scFab comprises an amino acid sequence selected from the group consisting of SEQ ID Nos: 75 and 77, even more preferably SEQ ID NO: 77.

Preferably, the CTHRC1 binding moiety of the invention is an scFv, or scFab comprising an amino acid sequence selected from the group consisting of any one of SEQ ID Nos: 76, 78, 80, 82, and 84, more preferably an scFv or scFab comprising an amino acid sequence selected from the group consisting of SEQ ID Nos: 87 and 84. Still more preferably, the scFv or scFab comprises an amino acid sequence selected from the group consisting of SEQ ID Nos: 76 and 78, even more preferably SEQ ID NO: 78.

Preferably, the CTHRC1 binding moiety of the invention is an scFv, or scFab comprising an amino acid sequence selected from the group consisting of any one of SEQ ID Nos: 75, 77, 79, 81, and 83, and any one of SEQ ID Nos: 76, 78, 80, 82, and 84. More preferably, the antigen binding moiety portion in the CAR of the invention is an scFv, or scFab comprising an amino acid sequence selected from the group consisting of any one of SEQ ID Nos: 77 and 83, and any one of SEQ ID Nos: 78 and 84. Still more preferably, the scFv or scFab comprises an amino acid sequence selected from the group consisting of SEQ ID Nos: 75 and 77 and an any one of SEQ ID Nos: 76 and 78, even more preferably SEQ ID NO: 77 and SEQ ID NO: 78.

In embodiments, the CTHRC1 binding moiety of the invention is an scFv, or scFab comprising an amino acid that is encoded by a nucleotide sequence selected from the group consisting of SEQ ID Nos: 88-97. In embodiments, the CTHRC1 binding moiety of the invention is an scFv, or scFab comprising an amino acid that is encoded by a nucleotide sequence selected from the group consisting of any one of SEQ ID Nos: 88, 90, 92, 94, and 96.

In embodiments, the CTHRC1 binding moiety of the invention is an scFv, or scFab comprising an amino acid that is encoded by a nucleotide sequence selected from the group consisting of any one of SEQ ID Nos: 89, 91, 93, 95, and 97.

In embodiments, the CTHRC1 binding moiety of the invention is an scFv, or scFab comprising an amino acid that is encoded by a nucleotide sequence selected from the group consisting of any one of SEQ ID Nos: 100, 102, 104, 106, and 108; and from the group consisting of any one of SEQ ID Nos: 101, 103, 105, 107, and 109.

In embodiments, the CTHRC1 binding moiety of the invention is an scFv, or scFab comprising an amino acid sequence selected from the group consisting of any CDR sequence in Table 4 and Table 5.

Preferably, the CTHRC1 binding moiety of the invention is an scFv, or scFab comprising an amino acid sequence selected from the group consisting of any CDR sequence in Table 4 and Table 5; and further comprises an amino acid sequence selected from the group consisting of any one of SEQ ID Nos: 75-84. More preferably, the CTHRC1 binding moiety of the invention is an scFv, or scFab comprising a heavy chain variable region comprising a CDR1 sequence selected from the group consisting of SEQ ID Nos: 45-49; a CDR2 sequence selected from the group consisting of SEQ ID Nos: 50-54; and a CDR3 sequence selected from the group consisting of SEQ ID Nos: 55-59, and a light chain variable region comprising a CDR1 sequence selected from the group consisting of SEQ ID Nos: 60-64; a CDR2 sequence selected from the group consisting of SEQ ID Nos: 65-69; and a CDR3 sequence selected from the group consisting of SEQ ID Nos: 70-74; and further comprises an amino acid sequence selected from the group consisting of any one of SEQ ID NOs: 75-84.

Preferably, the CTHRC1 binding moiety of the invention is an scFv or scFab comprising an amino acid sequence selected from the group consisting of SEQ ID Nos: 102, 104, 106, and 108, more preferably from the group consisting of SEQ ID Nos: 102 and 106, and most preferably SEQ ID NO: 102.

In a preferred embodiment, the CTHRC1 binding moiety comprises the a HCDR1 of SEQ ID NO: 46, a HCDR2 of SEQ ID NO: 51, a HCDR 3 of SEQ ID NO: 56, a LCDR1 of SEQ ID NO: 61, a LCDR2 of SEQ ID NO: 66, and a LCDR3 of SEQ ID NO: 71.

In another preferred embodiment, the CTHRC1 binding moiety comprises the a HCDR1 of SEQ ID NO: 45, a HCDR2 of SEQ ID NO: 50, a HCDR 3 of SEQ ID NO: 55, a LCDR1 of SEQ ID NO: 60, a LCDR2 of SEQ ID NO: 65, and a LCDR3 of SEQ ID NO: 70.

In some embodiments, the CTHRC1 binding moiety comprises the a HCDR1 of SEQ ID NO: 47, a HCDR2 of SEQ ID NO: 52, a HCDR 3 of SEQ ID NO: 57, a LCDR1 of SEQ ID NO: 62, a LCDR2 of SEQ ID NO: 67, and a LCDR3 of SEQ ID NO: 72.

In some embodiments, the CTHRC1 binding moiety comprises the a HCDR1 of SEQ ID NO: 48, a HCDR2 of SEQ ID NO: 53, a HCDR 3 of SEQ ID NO: 58, a LCDR1 of SEQ ID NO: 63, a LCDR2 of SEQ ID NO: 68, and a LCDR3 of SEQ ID NO: 73.

In some embodiments, the CTHRC1 binding moiety comprises the a HCDR1 of SEQ ID NO: 49, a HCDR2 of SEQ ID NO: 54, a HCDR 3 of SEQ ID NO: 59, a LCDR1 of SEQ ID NO: 64, a LCDR2 of SEQ ID NO: 69, and a LCDR3 of SEQ ID NO: 74.

In a preferred embodiment, the CTHRC1 binding moiety comprises a heavy chain variable domain comprising the sequence of SEQ ID NO: 77 and a light chain variable domain comprising the sequence of SEQ ID NO: 78.

In another preferred embodiment, the CTHRC1 binding moiety comprises a heavy chain variable domain comprising the sequence of SEQ ID NO: 75 and a light chain variable domain comprising the sequence of SEQ ID NO: 76.

In some embodiments, the CTHRC1 binding moiety comprises a heavy chain variable domain comprising the sequence of SEQ ID NO: 79 and a light chain variable domain comprising the sequence of SEQ ID NO: 80.

In some embodiments, the CTHRC1 binding moiety comprises a heavy chain variable domain comprising the sequence of SEQ ID NO: 81 and a light chain variable domain comprising the sequence of SEQ ID NO: 82.

In some embodiments, the CTHRC1 binding moiety comprises a heavy chain variable domain comprising the sequence of SEQ ID NO: 83 and a light chain variable domain comprising the sequence of SEQ ID NO: 84.

In any of the foregoing embodiments, the anti-CTHRC1 fusion protein can have a binding affinity for CTHRC1 of less than 10 nM, preferably less than 5 nM, more preferably less than 1 nM. The binding affinity of the anti-CTHRC1 fusion protein can, for example, be determined by the Scatchard analysis described in Munson et al., Anal. Biochem. 107: 220 (1980).

In embodiments, where the CTHRC1 binding moiety is an antibody, it can be a chimeric, humanized or human antibody. In embodiments, where the CTHRC1 binding moiety is an antibody, it can be a fragment of a full-length antibody, single-chain antibody, a single domain antibody (e.g. a heavy chain only antibody), a single-chain variable fragment (scFv), or any other type of “antibody” defined herein. Exemplary antibodies include monoclonal, chimeric, humanized, and human antibodies.

In embodiments, the CTHRC1 binding moiety can be a CTHRC1 binding moiety as described in any of WO 2010/047448, U.S. Patent Application Publication No. 2005/0147602, U.S. Patent Application Publication No. 2016/0000866, WO 2014/200134, U.S. Pat. No. 9,050,296, U.S. Patent Application Publication No. 2013/0190357, U.S. Patent Application Publication No. 2018/0313846, U.S. Pat. No. 9,718,878, U.S. Patent Application Publication No. 2022/0204599, WO 2021/063972, U.S. Patent Application Publication No. 2018/0221442, WO 2010/047448, CN110257389A, and JPWO2007123010, each of which is incorporated herein by reference in its entirety. Additional CTHRC1 binding moieties can include, by way of example but not limitation, 10G07 (Duarte et al., 2014 PLOS ONE, 9(6): e100449, incorporated herein by reference), 13D11, and 19C07, clone 13E09, anti-CTHRC1 antibody, H-213, incorporated herein by reference, anti-CTHRC1 antibody, T-19, which is incorporated herein by reference (Santa Cruz Biotechnology, Inc., Dallas, Tex.), anti-CTHRC1 antibodies: SAB1102667, HPA059806, SAB2107469, and SAB1402656, each of which is incorporated herein by reference (Sigma-Aldrich®, St. Louis, Mo.), and anti-CTHRC1 antibody PA5-38054, incorporated herein by reference (Thermo Scientific, Waltham, Mass.), Vli-55, an antibody or fragment thereof comprising HCDR1, HCDR2, and HCDR3 and LCDR1, LCDR2, and LCDR3 of SEQ ID Nos: 9-11 and 13-15, respectively of WO 2014/200134, and an antibody or fragment thereof comprising HCDR1, HCDR2, and HCDR2 and LCDR1, LCDR2, and LCDR3 of SEQ ID Nos: 1-3 or 11-13 (HCDRs) and 4-6 or 14-16 (LCDRs), respectively, and/or a heavy chain variable region of any of SEQ ID Nos: 7, 17, 21, or 25 and/or a light chain variable region of any of SEQ ID Nos: 9, 19, 23, or 27, including clones cCMAb45, cCMAb96, hCMAb45, and hCMab96, 6C1 and 1D1 off U.S. Patent Application Publication No. 2022/0204599.

### Monoclonal Antibodies

A monoclonal antibody (mAb) to an antigen-of-interest can be prepared by using any technique known in the art. These include, but are not limited to, the hybridoma technique originally described by Kohler and Milstein (1975, Nature 256, 495-497), the human B cell hybridoma technique (Kozbor et al., 1983, Immunology Today 4: 72), and the EBV-hybridoma technique (Cole et al., 1985, Monoclonal Antibodies and Cancer Therapy, Alan R. Liss, Inc., pp. 77-96). The Selected Lymphocyte Antibody Method (SLAM) (Babcook, J. S., et al., A novel strategy for generating monoclonal antibodies from single, isolated lymphocytes producing antibodies of defined specificities. Proc Natl Acad Sci USA, 1996. 93 (15): p. 7843-8.) and (McLean G et al., 2005, J. Immunol. 174(8): 4768-78. Such antibodies may be of any immunoglobulin class including IgG, IgM, IgE, IgA, and IgD and any subclass thereof. The hybridoma producing the mAbs of use in this invention may be cultivated in vitro or in vivo.

Monoclonal antibodies may be made using the hybridoma method first described by Kohler et al., Nature, 256: 495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which may contain one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133: 3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem. 107: 220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal, e.g., by intraperitoneal injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells.

Review articles on recombinant expression in bacteria of DNA encoding the antibody include Skerra et al., Curr. Opinion in Immunol. 5: 256-62 (1993) and Pluckthun, Immunol. Rev. 130: 151-88 (1992).

In a further embodiment, monoclonal antibodies or antibody fragments can be isolated from antibody phage libraries generated using the techniques described in McCafferty et al., Nature, 348: 552-54 (1990). Clackson et al., Nature, 352: 624-28 (1991) and Marks et al., J. Mol. Biol., 222: 581-97 (1991) describe the isolation of murine and human antibodies, respectively, using phage libraries. Subsequent publications describe the production of high affinity (nM range) human antibodies by chain shuffling (Marks et al., Bio/Technology, 10: 779-83 (1992)), as well as combinatorial infection and in vivo recombination as a strategy for constructing very large phage libraries (Waterhouse et al., Nuc. Acids. Res. 21: 2265-6 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (CH and CO sequences for the homologous murine sequences (U.S. Pat. No. 4,816,567; and Morrison, et al., Proc. Natl. Acad. Sci. USA, 81: 6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

### Chimeric, Humanized and Human Antibodies

In some embodiments, the CTHRC1 binding moiety is a chimeric antibody. Certain chimeric antibodies are described, e.g., in U.S. Pat. No. 4,816,567; and Morrison et al., Proc. Natl. Acad. Sci. USA, 81: 6851-5 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the CDR residues are derived), e.g., to restore or improve antibody specificity or affinity.

The anti-CTHRC1 antibodies of the invention may comprise humanized antibodies or human antibodies. Humanized forms of non-human (e.g., murine or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321: 522-5 (1986); Riechmann et al., Nature, 332: 323-9 (1988); and Presta, Curr. Op. Struct. Biol., 2: 593-6 (1992)).

A humanized antibody as a CTHRC1 binding moiety of the invention may comprise one or more human and/or human consensus non-hypervariable region (e.g., framework) sequences in its heavy and/or light chain variable domain. In some embodiments, one or more additional modifications are present within the human and/or human consensus non-hypervariable region sequences. In one embodiment, the heavy chain variable domain of an antibody of the invention comprises a human consensus framework sequence, which in one embodiment is the subgroup III consensus framework sequence. In one embodiment, an antibody of the invention comprises a variant subgroup III consensus framework sequence modified at at least one amino acid position.

As is known in the art, and as described in greater detail herein, the amino acid position/boundary delineating a hypervariable region of an antibody can vary, depending on the context and the various definitions known in the art (as described below). Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions (as further defined below). The invention provides antibodies comprising modifications in these hybrid hypervariable positions. In one embodiment, these hypervariable positions include one or more positions 26-30, 33-35B, 47-49, 57-65, 93, 94 and 101-102 in a heavy chain variable domain. In one embodiment, these hybrid hypervariable positions include one or more of positions 24-29, 35-36, 46-49, 56 and 97 in a light chain variable domain. In one embodiment, an antibody of the invention comprises a human variant human subgroup consensus framework sequence modified at one or more hybrid hypervariable positions.

A CTHRC1 binding moiety of the invention can comprise any suitable human or human consensus light chain framework sequences, provided the antibody exhibits the desired biological characteristics (e.g., a desired binding affinity). In one embodiment, an antibody of the invention comprises at least a portion (or all) of the framework sequence of human κ light chain. In one embodiment, a CTHRC1 binding moiety of the invention comprises at least a portion (or all) of human κ subgroup I framework consensus sequence.

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. Reduction or elimination of a HAMA response is a significant aspect of clinical development of suitable therapeutic agents (see, e.g., Khaxzaeli et al., J. Natl. Cancer Inst. (1988), 80:937; Jaffers et al., Transplantation (1986), 41:572; Shawler et al., J. Immunol. (1985), 135:1530; Sears et al., J. Biol. Response Mod. (1984), 3:138; Miller et al., Blood (1983), 62:988; Hakimi et al., J. Immunol. (1991), 147:1352; Reichmann et al., Nature (1988), 332:323; Junghans et al., Cancer Res. (1990), 50: 1495). As described herein, the invention provides antibodies that are humanized such that HAMA response is reduced or eliminated. Variants of these antibodies can further be obtained using routine methods known in the art, some of which are further described below. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151: 2296 (1993); Chothia et al., J. Mol. Biol., 196: 901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89: 4285 (1992); Presta et al., J. Immunol. 151: 2623 (1993)).

For example, an amino acid sequence from an antibody as described herein can serve as a starting (parent) sequence for diversification of the framework and/or hypervariable sequence(s). A selected framework sequence to which a starting hypervariable sequence is linked is referred to herein as an acceptor human framework. While the acceptor human frameworks may be from, or derived from, a human immunoglobulin (the VL and/or VH regions thereof), preferably the acceptor human frameworks are from, or derived from, a human consensus framework sequence as such frameworks that have been demonstrated to have minimal, or no, immunogenicity in human patients.

Where the acceptor is derived from a human immunoglobulin, one may optionally select a human framework sequence that is selected based on its homology to the donor framework sequence by aligning the donor framework sequence with various human framework sequences in a collection of human framework sequences, and select the most homologous framework sequence as the acceptor.

In one embodiment, human consensus frameworks herein are from, or derived from, VH subgroup III and/or VL kappa subgroup I consensus framework sequences.

While the acceptor may be identical in sequence to the human framework sequence selected, whether that be from a human immunoglobulin or a human consensus framework, the present invention contemplates that the acceptor sequence may comprise pre-existing amino acid substitutions relative to the human immunoglobulin sequence or human consensus framework sequence. These pre-existing substitutions are preferably minimal; usually four, three, two or one amino acid differences only relative to the human immunoglobulin sequence or consensus framework sequence.

Hypervariable region residues of the non-human antibody are incorporated into the VL and/or VH acceptor human frameworks. For example, one may incorporate residues corresponding to the Kabat CDR residues, the Chothia hypervariable loop residues, the Abm residues, and/or contact residues. Optionally, the extended hypervariable region residues as follows are incorporated: 24-34 (L1), 50-56 (L2) and 89-97 (L3), 26-35B (H1), 50-65, 47-65 or 49-65 (H2) and 93-102, 94-102, or 95-102 (H3).

While “incorporation” of hypervariable region residues is discussed herein, it will be appreciated that this can be achieved in various ways, for example, nucleic acid encoding the desired amino acid sequence can be generated by mutating nucleic acid encoding the mouse variable domain sequence so that the framework residues thereof are changed to acceptor human framework residues, or by mutating nucleic acid encoding the human variable domain sequence so that the hypervariable domain residues are changed to non-human residues, or by synthesizing nucleic acid encoding the desired sequence, etc.

As described herein, hypervariable region-grafted variants may be generated by Kunkel mutagenesis of nucleic acid encoding the human acceptor sequences, using a separate oligonucleotide for each hypervariable region. Kunkel et al., Methods Enzymol. 154:367-382 (1987). Appropriate changes can be introduced within the framework and/or hypervariable region, using routine techniques, to correct and re-establish proper hypervariable region-antigen interactions.

Phage(mid) display (also referred to herein as phage display in some contexts) can be used as a convenient and fast method for generating and screening many different potential variant antibodies in a library generated by sequence randomization. However, other methods for making and screening altered antibodies are available to the skilled person.

Phage(mid) display technology has provided a powerful tool for generating and selecting novel proteins which bind to a ligand, such as an antigen. Using the techniques of phage(mid) display allows the generation of large libraries of protein variants which can be rapidly sorted for those sequences that bind to a target molecule with high affinity. Nucleic acids encoding variant polypeptides are generally fused to a nucleic acid sequence encoding a viral coat protein, such as the gene III protein or the gene VIII protein. Monovalent phagemid display systems where the nucleic acid sequence encoding the protein or polypeptide is fused to a nucleic acid sequence encoding a portion of the gene III protein have been developed. (Bass, S., Proteins, 8:309 (1990); Lowman and Wells, Methods: A Companion to Methods in Enzymology, 3:205 (1991)). In a monovalent phagemid display system, the gene fusion is expressed at low levels and wild type gene III proteins are also expressed so that infectivity of the particles is retained.

Methods of generating peptide libraries and screening those libraries have been disclosed in many patents (e.g., U.S. Pat. Nos. 5,723,286, 5,432,018, 5,580,717, 5,427,908 and 5,498,530).

Libraries of antibodies or antigen binding polypeptides have been prepared in a number of ways including by altering a single gene by inserting random DNA sequences or by cloning a family of related genes. Methods for displaying antibodies or antigen binding fragments using phage(mid) display have been described in U.S. Pat. Nos. 5,750,373, 5,733,743, 5,837,242, 5,969,108, 6,172,197, 5,580,717, and 5,658,727. The library is then screened for expression of antibodies or antigen binding proteins with the desired characteristics.

Methods of substituting an amino acid of choice into a template nucleic acid are well established in the art, some of which are described herein. For example, hypervariable region residues can be substituted using the Kunkel method (e.g., Kunkel et al., Methods Enzymol. 154:367-382 (1987)).

The sequence of oligonucleotides includes one or more of the designed codon sets for the hypervariable region residues to be altered. A codon set is a set of different nucleotide triplet sequences used to encode desired variant amino acids. Codon sets can be represented using symbols to designate particular nucleotides or equimolar mixtures of nucleotides as shown in below according to the IUB code.

### IUB Codes

G Guanine
A Adenine
T Thymine
C Cytosine
R (A or G)
Y (C or T)
M (A or C)
K (G or T)
S (C or G)
W (A or T)
H (A or C or T)
B (C or G or T)
V (A or C or G)
D (A or G or T) H
N (A or C or G or T)

For example, in the codon set DVK, D can be nucleotides A or G or T; V can be A or G or C; and K can be G or T. This codon set can present 18 different codons and can encode amino acids Ala, Trp, Tyr, Lys, Thr, Asn, Lys, Ser, Arg, Asp, Glu, Gly, and Cys.

Oligonucleotide or primer sets can be synthesized using standard methods. A set of oligonucleotides can be synthesized, for example, by solid phase synthesis, containing sequences that represent all possible combinations of nucleotide triplets provided by the codon set and that will encode the desired group of amino acids. Synthesis of oligonucleotides with selected nucleotide “degeneracy” at certain positions is well known in that art. Such sets of nucleotides having certain codon sets can be synthesized using commercial nucleic acid synthesizers (available from, for example, Applied Biosystems, Foster City, Calif), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain nucleic acid template and also can include restriction enzyme sites for cloning purposes.

In one method, nucleic acid sequences encoding variant amino acids can be created by oligonucleotide-mediated mutagenesis. This technique is well known in the art as described by Zoller et al. Nucleic Acids Res. 10:6487-6504 (1987). Briefly, nucleic acid sequences encoding variant amino acids are created by hybridizing an oligonucleotide set encoding the desired codon sets to a DNA template, where the template is the single-stranded form of the plasmid containing a variable region nucleic acid template sequence. After hybridization, DNA polymerase is used to synthesize an entire second complementary strand of the template that will thus incorporate the oligonucleotide primer and will contain the codon sets as provided by the oligonucleotide set.

Generally, oligonucleotides of at least 25 nucleotides in length are used. An optimal oligonucleotide will have 12 to 15 nucleotides that are completely complementary to the template on either side of the nucleotide(s) coding for the mutation(s). This ensures that the oligonucleotide will hybridize properly to the single-stranded DNA template molecule. The oligonucleotides are readily synthesized using techniques known in the art such as that described by Crea et al., Proc. Nat'l. Acad. Sci. USA, 75:5765 (1978).

The DNA template is generated by those vectors that are either derived from bacteriophage M13 vectors (the commercially available M13 mp 18 and M13 mp 19 vectors are suitable), or those vectors that contain a single-stranded phage origin of replication as described by Viera et al., Meth. Enzymol., 153:3 (1987). Thus, the DNA that is to be mutated can be inserted into one of these vectors in order to generate single-stranded template. Production of the single-stranded template is described in sections 4.21-4.41 of Sambrook et al., above.

To alter the native DNA sequence, the oligonucleotide is hybridized to the single stranded template under suitable hybridization conditions. A DNA polymerizing enzyme, usually T7 DNA polymerase or the Klenow fragment of DNA polymerase I, is then added to synthesize the complementary strand of the template using the oligonucleotide as a primer for synthesis. A heteroduplex molecule is thus formed such that one strand of DNA encodes the mutated form of gene 1, and the other strand (the original template) encodes the native, unaltered sequence of gene 1. This heteroduplex molecule is then transformed into a suitable host cell, usually a prokaryote such as E. coli JM101. After growing the cells, they are plated onto agarose plates and screened using the oligonucleotide primer radiolabelled with a 32-Phosphate to identify the bacterial colonies that contain the mutated DNA.

The method described immediately above may be modified such that a homoduplex molecule is created wherein both strands of the plasmid contain the mutation(s). The modifications are as follows: The single stranded oligonucleotide is annealed to the single-stranded template as described above. A mixture of three deoxyribonucleotides, deoxyriboadenosine (dATP), deoxyriboguanosine (dGTP), and deoxyribothymidine (dTT), is combined with a modified thiodeoxyribocytosine called dCTP-(aS) (which can be obtained from Amersham). This mixture is added to the template-oligonucleotide complex. Upon addition of DNA polymerase to this mixture, a strand of DNA identical to the template except for the mutated bases is generated. In addition, this new strand of DNA will contain dCTP-(aS) instead of dCTP, which serves to protect it from restriction endonuclease digestion. After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell.

As indicated previously the sequence of the oligonucleotide set is of sufficient length to hybridize to the template nucleic acid and may also, but does not necessarily, contain restriction sites. The DNA template can be generated by those vectors that are either derived from bacteriophage M13 vectors or vectors that contain a single-stranded phage origin of replication as described by Viera et al. Meth. Enzymol., 153:3 (1987). Thus, the DNA that is to be mutated must be inserted into one of these vectors in order to generate single-stranded template. Production of the single-stranded template is described in sections 4.21-4.41 of Sambrook et al., supra.

According to another method, antigen binding may be restored during humanization of antibodies through the selection of repaired hypervariable regions (see, e.g., U.S. application Ser. No. 11/061,841, filed Feb. 18, 2005). The method includes incorporating non-human hypervariable regions onto an acceptor framework and further introducing one or more amino acid substitutions in one or more hypervariable regions without modifying the acceptor framework sequence. Alternatively, the introduction of one or more amino acid substitutions may be accompanied by modifications in the acceptor framework sequence.

According to another method, a library can be generated by providing upstream and downstream oligonucleotide sets, each set having a plurality of oligonucleotides with different sequences, the different sequences established by the codon sets provided within the sequence of the oligonucleotides. The upstream and downstream oligonucleotide sets, along with a variable domain template nucleic acid sequence, can be used in a polymerase chain reaction to generate a “library” of PCR products. The PCR products can be referred to as “nucleic acid cassettes”, as they can be fused with other related or unrelated nucleic acid sequences, for example, viral coat proteins and dimerization domains, using established molecular biology techniques.

The sequence of the PCR primers includes one or more of the designed codon sets for the solvent accessible and highly diverse positions in a hypervariable region. As described above, a codon set is a set of different nucleotide triplet sequences used to encode desired variant amino acids.

Antibody selectants that meet the desired criteria, as selected through appropriate screening/selection steps can be isolated and cloned using standard recombinant techniques.

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of a humanized anti-CTHRC1 antibody are contemplated. For example, the humanized antibody may be an antibody fragment, such as a Fab. Alternatively, the humanized antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge (see, e.g., Jakobovits et al., Proc. Natl. Acad. Sci. USA, 90: 2551 (1993); Jakobovits et al., Nature, 362: 255-8 (1993); Bruggemann et al., Year in Immuno. 7: 33 (1993); U.S. Pat. Nos. 5,545,806, 5,569,825, 5,591,669; 5,545,807; and WO 97/17852).

Alternatively, phage display technology (McCafferty et al., Nature 348: 552-53 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-97 (1991), or Griffith et al., EMBO J. 12: 725-34 (1993) (see also, U.S. Pat. Nos. 5,565,332 and 5,573,905).

As discussed above, human antibodies may also be generated by in vitro activated B cells (see, e.g., U.S. Pat. Nos. 5,567,610 and 5,229,275).

In another embodiment, the anti-CTHRC1 fusion proteins of this disclosure can include human monoclonal antibodies as the CTHRC1 binding moiety. Such human monoclonal antibodies directed against CTHRC1 can be generated using transgenic or transchromosomic mice carrying parts of the human immune system rather than the mouse system. These transgenic and transchromosomic mice include mice referred to herein as the HuMAb Mouse™ and KM Mouse™, respectively, and are collectively referred to herein as “human Ig mice.”

The HuMAb Mouse™ (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368(6474): 856-9). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N. et al. (1994), supra; reviewed in Lonberg, N. (1994) Handbook of Experimental Pharmacology 113: 49-101; Lonberg, N. and Huszar, D. (1995) Intern. Rev. Immunol. 13: 65-93, and Harding, F. and Lonberg, N. (1995) Ann. N.Y. Acad. Sci. 764: 536-46). Preparation and use of the HuMAb Mouse™, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90: 3720-4; Choi et al. (1993) Nature Genetics 4:117-23; Chen, J. et al. (1993) EMBO J. 12: 21-830; Tuaillon et al., (1994) J. Immunol. 152: 2912-20; Taylor, L. et al. (1994) International Immunology 6: 579-91; and Fishwild, D. et al. (1996) Nature Biotechnology 14: 845-51, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; 5,545,807; PCT Publication Nos. WO 92/03918, WO 93/12227, WO 94/25585, WO 97/13852, WO 98/24884 and WO 99/45962; and PCT Publication No. WO 01/14424.

In another embodiment, human antibodies of this disclosure can be raised using a mouse that carries human immunoglobulin sequences on transgenes and transchomosomes, such as a mouse that carries a human heavy chain transgene and a human light chain transchromosome. This mouse is referred to herein as a “KM Mouse™” and is described in detail in PCT Publication WO 02/43478.

Still further, alternative transgenic animal systems expressing human immunoglobulin genes are available in the art and can be used to raise anti-CTHRC1 antibodies of this disclosure. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963.

Moreover, alternative transchromosomic animal systems expressing human immunoglobulin genes are available in the art and can be used to raise anti-CTHRC1 antibodies of this disclosure. For example, mice carrying both a human heavy chain transchromosome and a human light chain tranchromosome, referred to as “TC mice” can be used; such mice are described in Tomizuka et al. (2000) Proc. Natl. Acad. Sci. USA 97: 722-7. As another example, cows carrying human heavy and light chain transchromosomes have been described in the art (e.g., Kuroiwa et al. (2002) Nature Biotechnology 20: 889-94 and PCT application No. WO 2002/092812) and can be used to raise anti-CTHRC1 antibodies of this disclosure. Additional examples of transgenic animals that can be used to produce anti-CTHCR1 antibodies include OmniRat™ and OmniMouse™ (see e.g., Osborn M., et al. (2013) Journal of Immunology 190: 1481-90; Ma B., et al. (2013) Journal of Immunological Methods 400-401: 78-86; Geurts A., et al. (2009) Science 325: 433, U.S. Pat. No. 8,907,157; European Pat. No. 2152880B1; European Pat. No. 2336329B1). Yet another example includes the use of VELOCIMMUNE® Technology (see, for example, U.S. Pat. No. 6,596,541, Regeneron Pharmaceuticals, VELOCIMMUNE®. Briefly, the VELOCIMMUNE® technology involves generation of a transgenic mouse having a genome comprising human heavy and light chain variable regions operably linked to endogenous mouse constant region loci such that the mouse produces an antigen-binding protein, e.g., antibody, comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

### Antibody Fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-7 (1992); and Brennan et al., Science, 229: 81 (1985)). However, these fragments can now be produced directly by recombinant host cells. Fab, Fv and scFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-7 (1992)). According to another approach, F(ab′)2 fragments can be isolated directly from recombinant host cell culture. Fab and F(ab′)2 fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv) (see WO 93/16185; U.S. Pat. Nos. 5,571,894; and 5,587,458). Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv (see Antibody Engineering, ed. Borrebaeck, supra. The antibody fragment may also be a “linear antibody”, e.g., as described in U.S. Pat. No. 5,641,870 for example.

### Antibody Variants and Modifications

### Substitution, Insertion and Deletion Variants

In addition to the anti-CTHRC1 antibodies described herein, it is contemplated that anti-CTHRC1 antibody variants can be prepared. Anti-CTHRC1 antibody variants can be prepared by introducing appropriate nucleotide changes into the encoding DNA, and/or by synthesis of the desired antibody or polypeptide. Those skilled in the art will appreciate that amino acid changes may alter post-translational processes of the anti-CTHRC1 antibody, such as changing the number or position of glycosylation sites or altering the membrane anchoring characteristics.

Variations in the anti-CTHRC1 antibodies described herein, can be made, for example, using any of the techniques and guidelines for conservative and non-conservative mutations set forth, for instance, in U.S. Pat. No. 5,364,934. Variations may be a substitution, deletion or insertion of one or more codons encoding the antibody or polypeptide that results in a change in the amino acid sequence as compared with the native sequence antibody or polypeptide. Optionally the variation is by substitution of at least one amino acid with any other amino acid in one or more of the domains of the anti-CTHRC1 antibody. Guidance in determining which amino acid residue may be inserted, substituted or deleted without adversely affecting the desired activity may be found by comparing the sequence of the anti-CTHRC1 antibody with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

Anti-CTHRC1 antibody fragments are provided herein. Such fragments may be truncated at the N-terminus or C-terminus, or may lack internal residues, for example, when compared with a full-length native antibody or protein. Certain fragments lack amino acid residues that are not essential for a desired biological activity of the anti-CTHRC1 antibody.

Anti-CTHRC1 antibody fragments may be prepared by any of a number of conventional techniques. Desired peptide fragments may be chemically synthesized. An alternative approach involves generating antibody or polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired antibody or polypeptide fragment, by polymerase chain reaction (PCR). Oligonucleotides that define the desired termini of the DNA fragment are employed at the 5′ and 3′ primers in the PCR. Preferably, anti-CTHRC1 antibody fragments share at least one biological and/or immunological activity with the native anti-CTHRC1 antibody disclosed herein.

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened.

TABLE 6

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in function or immunological identity of the anti-CTHRC1 antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

It should be understood that conservatively substituted versions of the anti-CTHRC1 fusion proteins or the first domain or second domain of the anti-CTHRC1 fusion proteins of the present disclosure are included within the scope of this disclosure.

The variations can be made using methods known in the art such as oligonucleotide-mediated (site-directed) mutagenesis, alanine scanning, and PCR mutagenesis. Site-directed mutagenesis (Carter et al., Nucl. Acids Res., 13: 4331 (1986); Zoller et al., Nucl. Acids Res., 10: 6487 (1987)), cassette mutagenesis (Wells et al., Gene, 34: 315 (1985)), restriction selection mutagenesis (Wells et al., Philos. Trans. R. Soc. London SerA, 317: 415 (1986)) or other known techniques can be performed on the cloned DNA to produce the anti-CTHRC1 antibody variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant (Cunningham and Wells, Science, 244: 1081-5 (1989)). Alanine is also typically preferred because it is the most common amino acid. Further, it is frequently found in both buried and exposed positions (Creighton, The Proteins, (W.H. N.Y.); Chothia, J. Mol. Biol., 150: 1 (1976)). If alanine substitution does not yield adequate amounts of variant, an isoteric amino acid can be used.

Any cysteine residue not involved in maintaining the proper conformation of the anti-CTHRC1 antibody also may be substituted, generally with serine, to improve the oxidative stability of the molecule and prevent aberrant crosslinking. Conversely, cysteine bond(s) may be added to the anti-CTHRC1 antibody to improve its stability (particularly where the antibody is an antibody fragment such as an Fv fragment).

A particularly preferred type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and CTHRC1 polypeptide. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the anti-CTHRC1 antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-CTHRC1 antibody.

### Modifications

Covalent modifications of anti-CTHRC1 antibodies are included within the scope of this invention. One type of covalent modification includes reacting targeted amino acid residues of an anti-CTHRC1 antibody with an organic derivatizing agent that is capable of reacting with selected side chains or the N- or C-terminal residues of the anti-CTHRC1 antibody. Derivatization with bifunctional agents is useful, for instance, for crosslinking anti-CTHRC1 antibody to a water-insoluble support matrix or surface for use in the method for purifying anti-CTHRC1 antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification of the anti-CTHRC1 antibody included within the scope of this invention comprises altering the native glycosylation pattern of the antibody or polypeptide. “Altering the native glycosylation pattern” is intended for purposes herein to mean deleting one or more carbohydrate moieties found in native sequence anti-CTHRC1 antibody (either by removing the underlying glycosylation site or by deleting the glycosylation by chemical and/or enzymatic means), and/or adding one or more glycosylation sites that are not present in the native sequence anti-CTHRC1 antibody. In addition, the phrase includes qualitative changes in the glycosylation of the native proteins, involving a change in the nature and proportions of the various carbohydrate moieties present.

Glycosylation of antibodies and other polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the anti-CTHRC1 antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original anti-CTHRC1 antibody (for O-linked glycosylation sites). The anti-CTHRC1 antibody amino acid sequence may optionally be altered through changes at the DNA level, particularly by mutating the DNA encoding the anti-CTHRC1 antibody at preselected bases such that codons are generated that will translate into the desired amino acids.

Another means of increasing the number of carbohydrate moieties on the anti-CTHRC1 antibody is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the anti-CTHRC1 antibody may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

### Fc Region Variants

It may be desirable to modify the antibody of the invention with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC) (see Caron et al., J. Exp Med. 176: 1191-5 (1992); Shopes, B. J. Immunol. 148: 2918-22 (1992). Homodimeric antibodies with enhanced anti-tumor activity may also be prepared using heterobifunctional cross-linkers as described in Wolff et al., Cancer Research 53: 2560-5 (1993). Alternatively, an antibody can be engineered which has dual Fc regions and may thereby have enhanced complement lysis and ADCC capabilities. See Stevenson et al., Anti-Cancer Drug Design 3: 219-30 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

### Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies can be generated as described, e.g, in U.S. Pat. No. 7,521,541.

### Certain Methods of Making Antibodies

Screening for Anti-CTHRC1 Antibodies with Desired Properties

Techniques for generating antibodies that bind to CTHRC1 polypeptides have been described above. One may further select antibodies with certain biological characteristics, as desired.

The growth inhibitory effects of an anti-CTHRC1 antibody of the invention may be assessed by methods known in the art, e.g., using cells which express a CTHRC1 polypeptide either endogenously or following transfection with the CTHRC1 gene. For example, appropriate tumor cell lines and CTHRC1-transfected cells may be treated with an anti-CTHRC1 monoclonal antibody of the invention at various concentrations for a few days (e.g., 2-7) days and stained with crystal violet or MTT or analyzed by some other colorimetric assay. Another method of measuring proliferation would be by comparing 3H-thymidine uptake by the cells treated in the presence or absence an anti-CTHRC1 antibody of the invention. After treatment, the cells are harvested and the amount of radioactivity incorporated into the DNA quantitated in a scintillation counter. Appropriate positive controls include treatment of a selected cell line with a growth inhibitory antibody known to inhibit growth of that cell line. Growth inhibition of tumor cells in vivo can be determined in various ways known in the art. The tumor cell may be one that overexpresses and/or displays a CTHRC1 polypeptide. The anti-CTHRC1 antibody will inhibit cell proliferation of a CTHRC1-displaying tumor cell in vitro or in vivo by about 25-100% compared to the untreated tumor cell, more preferably, by about 30-100%, and even more preferably by about 50-100% or 70-100%, in one embodiment, at an antibody concentration of about 0.5 to 30 g/mL. Growth inhibition can be measured at an antibody concentration of about 0.5 to 30 g/mL or about 0.5 nM to 200 nM in cell culture, where the growth inhibition is determined 1-10 days after exposure of the tumor cells to the antibody. The antibody is growth inhibitory in vivo if administration of the anti-CTHRC1 antibody at about 1 μg/kg to about 100 mg/kg body weight results in reduction in tumor size or reduction of tumor cell proliferation within about 5 days to 3 months from the first administration of the antibody, preferably within about 5 to 30 days.

To select for an anti-CTHRC1 antibody which induces cell death, loss of membrane integrity as indicated by, e.g., propidium iodide (PI), trypan blue or 7AAD uptake may be assessed relative to control. A PI uptake assay can be performed in the absence of complement and immune effector cells. CTHRC1 polypeptide-displaying tumor cells are incubated with medium alone or medium containing the appropriate anti-CTHRC1 antibody (e.g, at about 10 g/mL). The cells are incubated for a 3 day time period. Following each treatment, cells are washed and aliquoted into 35 mm strainer-capped 12×75 tubes (1 mL per tube, 3 tubes per treatment group) for removal of cell clumps. Tubes then receive PI (10 g/mL). Samples may be analyzed using a FACSCAN® flow cytometer and FACSCONVERT® CellQuest software (Becton Dickinson). Those anti-CTHRC1 antibodies that induce statistically significant levels of cell death as determined by PI uptake may be selected as cell death-inducing anti-CTHRC1 antibodies.

To screen for antibodies which bind to an epitope on a CTHRC1 polypeptide bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody binds the same site or epitope as a known anti-CTHRC1 antibody. Alternatively, or additionally, epitope mapping can be performed by methods known in the art. For example, the antibody sequence can be mutagenized such as by alanine scanning, to identify contact residues. The mutant antibody is initially tested for binding with polyclonal antibody to ensure proper folding. In a different method, peptides corresponding to different regions of a CTHRC1 polypeptide can be used in competition assays with the test antibodies or with a test antibody and an antibody with a characterized or known epitope.

In addition, candidate antibodies may also be screened for function using one or more of the following: in vivo screening for inhibition of metastasis, inhibition of chemotaxis by an in vitro method (e.g., U.S. 2010/0061978, incorporated herein by reference in its entirety), inhibition of vascularization, inhibition of tumor growth, and decrease in tumor size.

### Certain Library Screening Methods

Anti-CTHRC1 antibodies of the invention can be made by using combinatorial libraries to screen for antibodies with the desired activity or activities. For example, a variety of methods are known in the art for generating phage display libraries and screening such libraries for antibodies possessing the desired binding characteristics. Such methods are described generally in Hoogenboom et al. (2001) in Methods in Molecular Biology 178: 1-37 (O'Brien et al., ed., Human Press, Totowa, NJ), and in certain embodiments, in Lee et al. (2004) J. Mol. Biol. 340: 1073-93.

In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the anti-CTHRC1 antibodies of the invention can be obtained by designing a suitable antigen screening procedure to select for the phage clone of interest followed by construction of a full length anti-CTHRC1 antibody clone using the Fv sequences from the phage clone of interest and suitable constant region (Fc) sequences described in Kabat et al., Sequences of Proteins of Immunological Interest, Fifth Edition, NIH Publication 91-3242, Bethesda MD (1991), vols. 1-3.

In certain embodiments, the antigen-binding domain of an antibody is formed from two variable (V) regions of about 110 amino acids, one each from the light (VL) and heavy (VH) chains, that both present three hypervariable loops (HVRs) or complementarity-determining regions (CDRs). Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described in Winter et al., Ann. Rev. Immunol., 12: 433-55 (1994). As used herein, scFv encoding phage clones and Fab encoding phage clones are collectively referred to as “Fv phage clones” or “Fv clones.”

Repertoires of VH and VL genes can be separately cloned by polymerase chain reaction (PCR) and recombined randomly in phage libraries, which can then be searched for antigen-binding clones as described in Winter et al., Ann. Rev. Immunol., 12: 433-55 (1994). Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12: 725-34 (1993). Finally, naive libraries can also be made synthetically by cloning the unrearranged V-gene segments from stem cells and using PCR primers containing random sequence to encode the highly variable CDR3 regions and to accomplish rearrangement in vitro as described by Hoogenboom and Winter, J. Mol. Biol., 227: 381-8 (1992).

In certain embodiments, filamentous phage is used to display antibody fragments by fusion to the minor coat protein pIII. The antibody fragments can be displayed as single chain Fv fragments, in which VH and VL domains are connected on the same polypeptide chain by a flexible polypeptide spacer, e.g., as described by Marks et al., J. Mol. Biol., 222: 581-97 (1991), or as Fab fragments, in which one chain is fused to pIII and the other is secreted into the bacterial host cell periplasm where assembly of a Fab-coat protein structure which becomes displayed on the phage surface by displacing some of the wild type coat proteins, e.g., as described in Hoogenboom et al., Nucl. Acids Res., 19: 4133-7 (1991).

In general, nucleic acids encoding antibody gene fragments are obtained from immune cells harvested from humans or animals. If a library biased in favor of anti-CTHRC1 clones is desired, the subject is immunized with CTHRC1 to generate an antibody response, and spleen cells and/or circulating B cells other peripheral blood lymphocytes (PBLs) are recovered for library construction. In some embodiments, a human antibody gene fragment library biased in favor of anti-CTHRC1 clones is obtained by generating an anti-CTHRC1 antibody response in transgenic mice carrying a functional human immunoglobulin gene array (and lacking a functional endogenous antibody production system) such that CTHRC1 immunization gives rise to B cells producing human antibodies against CTHRC1. The generation of human antibody-producing transgenic mice is described below.

Additional enrichment for anti-CTHRC1 reactive cell populations can be obtained by using a suitable screening procedure to isolate B cells expressing CTHRC1-specific membrane bound antibody, e.g., by cell separation using CTHRC1 affinity chromatography or adsorption of cells to fluorochrome-labeled CTHRC1 followed by flow-activated cell sorting (FACS).

Alternatively, the use of spleen cells and/or B cells or other PBLs from an unimmunized donor provides a better representation of the possible antibody repertoire, and also permits the construction of an antibody library using any animal (human or non-human) species in which CTHRC1 is not antigenic. For libraries incorporating in vitro antibody gene construction, stem cells are harvested from the subject to provide nucleic acids encoding unrearranged antibody gene segments. The immune cells of interest can be obtained from a variety of animal species, such as human, mouse, rat, lagomorpha, luprine, canine, feline, porcine, bovine, equine, and avian species, etc.

Nucleic acid encoding antibody variable gene segments (including VH and VL segments) are recovered from the cells of interest and amplified. In the case of rearranged VH and VL gene libraries, the desired DNA can be obtained by isolating genomic DNA or mRNA from lymphocytes followed by polymerase chain reaction (PCR) with primers matching the 5′ and 3′ ends of rearranged VH and VL genes as described in Orlandi et al., Proc. Natl. Acad. Sci. (USA), 86: 3833-7 (1989), thereby making diverse V gene repertoires for expression.

The V genes can be amplified from cDNA and genomic DNA, with back primers at the 5′ end of the exon encoding the mature V-domain and forward primers based within the J-segment as described in Orlandi et al. (1989) and in Ward et al., Nature, 341: 544-6 (1989). However, for amplifying from cDNA, back primers can also be based in the leader exon as described in Jones et al., Biotechnol., 9: 88-9 (1991), and forward primers within the constant region as described in Sastry et al., Proc. Natl. Acad. Sci. (USA), 86: 5728-32 (1989). To maximize complementarity, degeneracy can be incorporated in the primers as described in Orlandi et al. (1989) or Sastry et al. (1989). In certain embodiments, library diversity is maximized by using PCR primers targeted to each V-gene family in order to amplify all available VH and VL arrangements present in the immune cell nucleic acid sample, e.g., as described in the method of Marks et al., J. Mol. Biol., 222: 581-97 (1991) or as described in the method of Orum et al., Nucleic Acids Res., 21: 4491-98 (1993). For cloning of the amplified DNA into expression vectors, rare restriction sites can be introduced within the PCR primer as a tag at one end as described in Orlandi et al. (1989), or by further PCR amplification with a tagged primer as described in Clackson et al., Nature, 352: 624-628 (1991).

Repertoires of synthetically rearranged V genes can be derived in vitro from V gene segments. Most of the human VH-gene segments have been cloned and sequenced (reported in Tomlinson et al., J. Mol. Biol., 227: 776-98 (1992)), and mapped (reported in Matsuda et al., Nature Genet., 3: 88-94 (1993); these cloned segments (including all the major conformations of the H1 and H2 loop) can be used to generate diverse VH gene repertoires with PCR primers encoding H3 loops of diverse sequence and length as described in Hoogenboom and Winter, J. Mol. Biol., 227: 381-388 (1992). VH repertoires can also be made with all the sequence diversity focused in a long H3 loop of a single length as described in Barbas et al., Proc. Natl. Acad. Sci. USA, 89: 4457-61 (1992). Human Vκ and Vλ segments have been cloned and sequenced (reported in Williams and Winter, Eur. J. Immunol., 23: 1456-61 (1993)) and can be used to make synthetic light chain repertoires. Synthetic V gene repertoires, based on a range of VH and VL folds, and L3 and H3 lengths, will encode antibodies of considerable structural diversity. Following amplification of V-gene encoding DNAs, germline V-gene segments can be rearranged in vitro according to the methods of Hoogenboom and Winter, J. Mol. Biol., 227: 381-8 (1992).

Repertoires of antibody fragments can be constructed by combining VH and VL gene repertoires together in several ways. Each repertoire can be created in different vectors, and the vectors recombined in vitro, e.g., as described in Hogrefe et al., Gene, 128: 119-26 (1993), or in vivo by combinatorial infection, e.g., the loxP system described in Waterhouse et al., Nucl. Acids Res., 21: 2265-66 (1993). The in vivo recombination approach exploits the two-chain nature of Fab fragments to overcome the limit on library size imposed by E. coli transformation efficiency. Naive VH and VL repertoires are cloned separately, one into a phagemid and the other into a phage vector. The two libraries are then combined by phage infection of phagemid-containing bacteria so that each cell contains a different combination and the library size is limited only by the number of cells present (about 1012 clones). Both vectors contain in vivo recombination signals so that the VH and VL genes are recombined onto a single replicon and are co-packaged into phage virions. These huge libraries provide large numbers of diverse antibodies of good affinity (Kd-1 of about 10-8 M).

Alternatively, the repertoires may be cloned sequentially into the same vector, e.g., as described in Barbas et al., Proc. Natl. Acad. Sci. USA, 88: 7978-7982 (1991), or assembled together by PCR and then cloned, e.g., as described in Clackson et al., Nature, 352: 624-628 (1991). PCR assembly can also be used to join VH and VL DNAs with DNA encoding a flexible peptide spacer to form single chain Fv (scFv) repertoires. In yet another technique, “in cell PCR assembly” is used to combine VH and VL genes within lymphocytes by PCR and then clone repertoires of linked genes as described in Embleton et al., Nucl. Acids Res., 20: 3831-3837 (1992).

The antibodies produced by naive libraries (either natural or synthetic) can be of moderate affinity (Kd-1 of about 10⁶ to 10⁷ M-1), but affinity maturation can also be mimicked in vitro by constructing and reselecting from secondary libraries as described in Winter et al. (1994), supra. For example, mutation can be introduced at random in vitro by using error-prone polymerase (reported in Leung et al., Technique, 1: 11-5 (1989)) in the method of Hawkins et al., J. Mol. Biol., 226: 889-96 (1992) or in the method of Gram et al., Proc. Natl. Acad. Sci USA, 89: 3576-80 (1992). Additionally, affinity maturation can be performed by randomly mutating one or more CDRs, e.g., using PCR with primers carrying random sequence spanning the CDR of interest, in selected individual Fv clones and screening for higher affinity clones. WO 9607754 described a method for inducing mutagenesis in a complementarity determining region of an immunoglobulin light chain to create a library of light chain genes. Another effective approach is to recombine the VH or VL domains selected by phage display with repertoires of naturally occurring V domain variants obtained from unimmunized donors and screen for higher affinity in several rounds of chain reshuffling as described in Marks et al., Biotechnol., 10: 779-83 (1992). This technique allows the production of antibodies and antibody fragments with affinities of about 10-9 M or less.

Screening of the libraries can be accomplished by various techniques known in the art. For example, CTHRC1 can be used to coat the wells of adsorption plates, expressed on host cells affixed to adsorption plates or used in cell sorting, or conjugated to biotin for capture with streptavidin-coated beads, or used in any other method for panning phage display libraries.

The phage library samples are contacted with immobilized CTHRC1 under conditions suitable for binding at least a portion of the phage particles with the adsorbent. Normally, the conditions, including pH, ionic strength, temperature and the like are selected to mimic physiological conditions. The phages bound to the solid phase are washed and then eluted by acid, e.g., as described in Barbas et al., Proc. Natl. Acad. Sci USA, 88: 7978-82 (1991), or by alkali, e.g., as described in Marks et al., J. Mol. Biol., 222: 581-97 (1991), or by CTHRC1 antigen competition, e.g., in a procedure similar to the antigen competition method of Clackson et al., Nature, 352: 624-8 (1991). Phages can be enriched 20 to 1,000-fold in a single round of selection. Moreover, the enriched phages can be grown in bacterial culture and subjected to further rounds of selection.

The efficiency of selection depends on many factors, including the kinetics of dissociation during washing, and whether multiple antibody fragments on a single phage can simultaneously engage with antigen. Antibodies with fast dissociation kinetics (and weak binding affinities) can be retained by use of short washes, multivalent phage display and high coating density of antigen in solid phase. The high density not only stabilizes the phage through multivalent interactions but favors rebinding of phage that has dissociated. The selection of antibodies with slow dissociation kinetics (and good binding affinities) can be promoted by use of long washes and monovalent phage display as described in Bass et al., Proteins, 8: 309-314 (1990) and in WO 92/09690, and a low coating density of antigen as described in Marks et al., Biotechnol., 10: 779-783 (1992).

It is possible to select between phage antibodies of different affinities, even with affinities that differ slightly, for CTHRC1. However, random mutation of a selected antibody (e.g., as performed in some affinity maturation techniques) is likely to give rise to many mutants, most binding to antigen, and a few with higher affinity. With limiting CTHRC1, rare high affinity phage could be competed out. To retain all higher affinity mutants, phages can be incubated with excess biotinylated CTHRC1, but with the biotinylated CTHRC1 at a concentration of lower molarity than the target molar affinity constant for CTHRC1. The high affinity-binding phages can then be captured by streptavidin-coated paramagnetic beads. Such “equilibrium capture” allows the antibodies to be selected according to their affinities of binding, with sensitivity that permits isolation of mutant clones with as little as two-fold higher affinity from a great excess of phages with lower affinity. Conditions used in washing phages bound to a solid phase can also be manipulated to discriminate on the basis of dissociation kinetics.

Anti-CTHRC1 clones may be selected based on activity. In certain embodiments, the invention provides anti-CTHRC1 antibodies that bind to living cells that naturally express CTHRC1. In one embodiment, the invention provides anti-CTHRC1 antibodies that block the binding between a CTHRC1 ligand and CTHRC1, but do not block the binding between a CTHRC1 ligand and a second protein. Fv clones corresponding to such anti-CTHRC1 antibodies can be selected by (1) isolating anti-CTHRC1 clones from a phage library as described above, and optionally amplifying the isolated population of phage clones by growing up the population in a suitable bacterial host; (2) selecting CTHRC1 and a second protein against which blocking and non-blocking activity, respectively, is desired; (3) adsorbing the anti-CTHRC1 phage clones to immobilized CTHRC1; (4) using an excess of the second protein to elute any undesired clones that recognize CTHRC1-binding determinants which overlap or are shared with the binding determinants of the second protein; and (5) eluting the clones which remain adsorbed following step (4). Optionally, clones with the desired blocking/non-blocking properties can be further enriched by repeating the selection procedures described herein one or more times.

DNA encoding hybridoma-derived monoclonal antibodies or phage display Fv clones of the invention is readily isolated and sequenced using conventional procedures (e.g., by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired monoclonal antibodies in the recombinant host cells. Review articles on recombinant expression in bacteria of antibody-encoding DNA include Skerra et al., Curr. Opinion in Immunol. 5: 256 (1993) and Pluckthun, Immunol. Rev. 130: 151 (1992).

DNA encoding the Fv clones of the invention can be combined with known DNA sequences encoding heavy chain and/or light chain constant regions (e.g., the appropriate DNA sequences can be obtained from Kabat et al., supra) to form clones encoding full or partial length heavy and/or light chains. It will be appreciated that constant regions of any isotype can be used for this purpose, including IgG, IgM, IgA, IgD, and IgE constant regions, and that such constant regions can be obtained from any human or animal species. An Fv clone derived from the variable domain DNA of one animal (such as human) species and then fused to constant region DNA of another animal species to form coding sequence(s) for “hybrid,” full length heavy chain and/or light chain is included in the definition of “chimeric” and “hybrid” antibody as used herein. In certain embodiments, an Fv clone derived from human variable DNA is fused to human constant region DNA to form coding sequence(s) for full- or partial-length human heavy and/or light chains.

DNA encoding anti-CTHRC1 antibody derived from a hybridoma can also be modified, for example, by substituting the coding sequence for human heavy- and light-chain constant domains in place of homologous murine sequences derived from the hybridoma clone (e.g., as in the method of Morrison et al., Proc. Natl. Acad. Sci. USA, 81: 6851-5 (1984)). DNA encoding a hybridoma- or Fv clone-derived antibody or fragment can be further modified by covalently joining to the immunoglobulin coding sequence all or part of the coding sequence for a non-immunoglobulin polypeptide. In this manner, “chimeric” or “hybrid” antibodies are prepared that have the binding specificity of the Fv clone or hybridoma clone-derived antibodies of the invention.

### Generation of Antibodies Using CAR-T Cells

Anti-CTHRC1 antibodies of the invention can be made by using CAR T-cell platforms to screen for antibodies with the desired activity or activities. Chimeric antigen receptors (CARs) are composed of an extracellular antigen recognition domain (usually a single-chain variable fragment (scFv) antibody) attached to transmembrane and cytoplasmic signaling domains. Alvarez-Vallina, L, Curr Gene Ther 1: 385-97 (2001). CAR-mediated recognition converts tumor-associated antigens (TAA) expressed on the cell surface into recruitment points of effector functions, addressing the goal of major histocompatibility complex-independent activation of effector cells. First-generation CARs were constructed through the fusion of a scFv-based TAA-binding domain to a cytoplasmic signaling domain typically derived either from the ζ chain of the T cell receptor (TCR)/CD3 complex or from the γ chain associated with some Fc receptors (Gross, G. et al., Proc Natl Acad Sci USA 86: 10024-8 (1989)). Second-generation CARs (CARv2) comprising the signaling region of the TCR ζ in series with the signaling domain derived from the T-cell co-stimulatory receptors CD28, 4-1BB (CD137) or OX40 (CD134) have also been developed (Sanz, L. et al., Trends Immunol 25: 85-91 (2004)). Third-generation CARs further combine the signaling potential of two costimulatory domains (e.g., both CD28 and 4-1BB) (Subklewe, M., et al., Transfus Med Hemother 46(1): 15-24 (2019).

Upon encountering antigen, the interaction of a genetically transferred CAR triggers effector functions and can mediate cytolysis of tumor cells. The utility and effectiveness of the CAR approach have been demonstrated in a variety of animal models, and ongoing clinical trials using CAR-based genetically engineered T lymphocytes for the treatment of cancer patients. Lipowska-Bhalla, G. et al., Cancer Immunol Immunother 61: 953-62 (2012). CARs enable targeting of effector cells toward any native extracellular antigen for which a suitable antibody exists. Engineered cells can be targeted not only to proteins but also to structures such as carbohydrate and glycolipid tumor antigens (Mezzanzanica, D. et al., Cancer Gene Ther 5: 401-7 (1998); Kershaw, M H. et al., Nat Rev Immunol 5: 928-40 (2005)).

Current methods for the generation of recombinant antibodies are mainly based on the use of purified proteins. Hoogenboom, H. R. et al., Nat Biotechnol 23: 1105-1116 (2005). However, a mammalian cell-based antibody display platform has recently been described, which takes advantage of the functional capabilities of T lymphocytes. Alonso-Camino et al, Molecular Therapy Nucleic Acids (2013) 2, e93. The display of antibodies on the surface of T lymphocytes, as a part of a CAR-mediating signaling, may ideally link the antigen-antibody interaction to a demonstrable change in cell phenotype, due to the surface expression of activation markers. Alonso-Camino, V. et al., PLoS ONE 4: e7174 (2009). By using a scFv-based CAR that recognizes a TAA, it has been demonstrated that combining CAR-mediated activation with fluorescence-activated cell sorting (FACS) of CD69+ T cells makes it possible to isolate binders to surface TAA, with an enrichment factor of at least 10³-fold after two rounds, resulting in a homogeneous population of T cells expressing TAA-specific CAR. Alonso-Camino, V, et al., PLoS ONE 4: e7174 (2009).

### Preparation of Anti-CTHRC1 Antibodies

The description below relates primarily to production of anti-CTHRC1 antibodies by culturing cells transformed or transfected with a vector containing anti-CTHRC1 antibody-encoding nucleic acid. It is, of course, contemplated that alternative methods, which are well known in the art, may be employed to prepare anti-CTHRC1 antibodies. For instance, the appropriate amino acid sequence, or portions thereof, may be produced by direct peptide synthesis using solid-phase techniques (e.g., Stewart et al., Solid-Phase Peptide Synthesis, W.H. Freeman Co., San Francisco, CA (1969); Merrifield, J. Am. Chem. Soc., 85:2149-2154 (1963)). In vitro protein synthesis may be performed using manual techniques or by automation. Automated synthesis may be accomplished, for instance, using an Applied Biosystems Peptide Synthesizer (Foster City, CA) using manufacturer's instructions. Various portions of the anti-CTHRC1 antibody may be chemically synthesized separately and combined using chemical or enzymatic methods to produce the desired anti-CTHRC1 antibody.

### Isolation of DNA Encoding Anti-CTHRC1 Antibody

DNA encoding anti-CTHRC1 antibody may be obtained from a cDNA library prepared from tissue believed to possess the anti-CTHRC1 antibody mRNA and to express it at a detectable level. Accordingly, human anti-CTHRC1 antibody DNA can be conveniently obtained from a cDNA library prepared from human tissue. The anti-CTHRC1 antibody-encoding gene may also be obtained from a genomic library or by known synthetic procedures (e.g., automated nucleic acid synthesis).

Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding anti-CTHRC1 antibody is to use PCR methodology (Sambrook et al., supra; Dieffenbach et al., PCR Primer: A Laboratory Manual (Cold Spring Harbor Laboratory Press, 1995)).

Techniques for screening a cDNA library are well known in the art. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like 32P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

### Selection and Transformation of Host Cells

Host cells are transfected or transformed with expression or cloning vectors described herein for anti-CTHRC1 antibody production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (TRL Press, 1991) and Sambrook et al., supra.

### Linkers

In any of the foregoing embodiments, the anti-CTHRC1 fusion protein can further comprise a peptide linker positioned between the first domain and the second domain. In certain aspects, the peptide linker directly links the first domain to the second domain. It should be understood that the first domain and the second domain can be positioned in at either end of the peptide linker. For example, the anti-CTHRC1 fusion protein can comprise (First domain-Linker-Second Domain) or (Second domain-Linker-First Domain).

In any of the foregoing embodiments, the peptide linker can be any suitable linker. By way of example, but not limitation, a peptide linker can include (G₄S)ₙ, (GSG)ₙ, (SG₄)ₙ, G₄(SG₄)ₙ peptide linkers where n is from 1 to 4. Preferably, the linker has the amino acid sequence of SEQ ID NO: 100 ((G₄S)₃).

Additional linkers are also described in U.S. Pat. No. 11,053,293 and U.S. Patent Application Publication No. 2019/0016771, each of which is incorporated herein by reference in its entirety. Further exemplary linkers can include GGGGSGGGGS (SEQ ID NO: 109), SGGGGSGGGG (SEQ ID NO: 110), GGGGSGGGGSGGGG or G₄(SG₄)₂ (SEQ ID NO: 111), and (G₄S)₄ or GGGGSGGGGSGGGGSGGGGS (SEQ ID NO: 112), GSPGSSSSGS (SEQ ID NO: 113), GSGSGSGS (SEQ ID NO: 114), GSGSGNGS (SEQ ID NO: 115), GGSGSGSG (SEQ ID NO: 116), GGSGSG (SEQ ID NO: 117), GGSG (SEQ ID NO: 118), GGSGNGSG (SEQ ID NO: 119), GGNGSGSG (SEQ ID NO: 120) and GGNGSG (SEQ ID NO: 121).

Alternative methods and designs for constructing anti-CTHRC1 fusion proteins of the present disclosure are provided in U.S. Pat. No. 10,392,445, which is incorporated herein by reference.

### Pharmaceutical Compositions

In embodiments, the anti-CTHRC1 fusion protein of the present disclosure can be formulated into a pharmaceutically acceptable composition that can include the anti-CTHRC1 fusion protein and a pharmaceutically acceptable carrier.

The anti-CTHRC1 fusion proteins and compositions thereof of the invention may be administered by any route appropriate to the condition to be treated. The fusion protein will typically be administered parenterally, i.e. infusion, subcutaneous, intramuscular, intravenous, intradermal, intrathecal and epidural.

For treating these cancers, in one embodiment, the antibody is administered via intravenous infusion. The dosage administered via infusion is in the range of about 0.001 mg/kg to about 100 mg/kg per dose by the subject's body weight, generally one dose per week for a total of one, two, three or four doses. Alternatively, the dosage range is of about 0.01 mg/kg to about 100 mg/kg, about 0.1 mg/kg to about 100 mg/kg, about 1 mg/kg to about 100 mg/kg, about 0.001 mg/kg to about 50 mg/kg, about 0.01 mg/kg to about 50 mg/kg, about 0.1 mg/kg to about 50 mg/kg, about 1 mg/kg to about 50 mg/kg, about 0.001 mg/kg to about 10 mg/kg, about 0.01 mg/kg to about 10 mg/kg, about 0.1 mg/kg to about 10 mg/kg, about 1 mg/kg to about 10 mg/kg, about 0.001 mg/kg to about 5 mg/kg, about 0.01 mg/kg to about 5 mg/kg, about 0.1 mg/kg to about 5 mg/kg, about 1 mg/kg to about 5 mg/kg, about 0.001 mg/kg to about 1 mg/kg, about 0.01 mg/kg to about 1 mg/kg, and about 0.1 mg/kg to about 1 mg/kg. The dose may be administered once per day, once per week, multiple times per week, but less than once per day, multiple times per month but less than once per day, multiple times per month but less than once per week, once per month or intermittently to relieve or alleviate symptoms of the disease. Administration may continue at any of the disclosed intervals until remission of the tumor or symptoms of the cancer being treated. Administration may continue after remission or relief of symptoms is achieved where such remission or relief is prolonged by such continued administration.

Therapeutic formulations comprising an anti-CTHRC1 fusion protein used in accordance with the present invention are prepared for storage by mixing the anti-CTHRC1 fusion protein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG). Pharmaceutical formulations to be used for in vivo administration are generally sterile. This is readily accomplished by filtration through sterile filtration membranes.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated immunoglobulins remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

A anti-CTHRC1 fusion protein of the present disclosure may be formulated in any suitable form for delivery to a target cell/tissue. For example, anti-CTHRC1 fusion proteins may be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA 82:3688 (1985); Hwang et al., Proc. Natl Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257: 286-8 (1982) via a disulfide interchange reaction. A chemotherapeutic agent is optionally contained within the liposome (See Gabizon et al., J. National Cancer Inst. 81(19): 1484 (1989)).

The formulations to be used for in vivo administration must be sterile. This is readily accomplished by filtration through sterile filtration membranes.

### Methods of Manufacture

Conventional methods for the production of fusion proteins are known in the art.

Methods of eukaryotic cell transfection and prokaryotic cell transformation, which means introduction of DNA into the host so that the DNA is replicable, either as an extrachromosomal or by chromosomal integrant, are known to the ordinarily skilled artisan, for example, CaCl2), CaPO4, liposome-mediated, polyethylene-glycol/DMSO and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

In some embodiments, a polynucleotide is provided that encodes an anti-CTHRC1 fusion protein of the present disclosure. In some embodiments, the polynucleotide encoding the anti-CTHRC1 fusion protein can be incorporated into a vector suitable for expressing the anti-CTHRC1 fusion protein in host cells.

The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The anti-CTHRC1 fusion protein may be produced recombinantly and can further include heterologous polypeptide, which may be a signal sequence or other polypeptide having a specific cleavage site at the N-terminus of the mature protein or polypeptide, or can be an affinity tag such a 6×His tag. In general, the signal sequence may be a component of the vector, or it may be a part of the anti-CTHRC1 fusion protein-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

For recombinant production of a fusion protein of the invention, the nucleic acid (e.g., cDNA or genomic DNA) encoding it is isolated and inserted into a replicable vector for further cloning (amplification of the DNA) or for expression. DNA encoding the fusion protein is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin. Suitable host cells for cloning or expressing the DNA in the vectors herein include prokaryote, yeast, or higher eukaryote cells.

The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

An anti-CTHRC1 fusion protein may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the anti-CTHRC1 antibody-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders. In embodiments, the anti-CTHRC1 fusion protein can be produced with a tag, such as a 6×His tag.

### Vectors

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In brief summary, the expression of natural or synthetic nucleic acids encoding CARs is typically achieved by operably linking a nucleic acid encoding the anti-CTHRC1 fusion protein or portions thereof to a promoter and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

In addition to the methods described above, the following methods may be used.

The expression constructs of the present invention may also be used for nucleic acid immunization and gene therapy, using standard gene delivery protocols. Methods for gene delivery are known in the art (e.g., U.S. Pat. Nos. 5,399,346, 5,580,859, 5,589,466, incorporated by reference herein in their entireties). In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

In order to assess the expression of an anti-CTHRC1 fusion protein or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the mi'imal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20.degree. C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Prokaryotic Host Cells

Polynucleotide sequences encoding polypeptide components of the antibody of the invention can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts.

Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides.

In general, plasmid vectors containing replicon and control sequences which are derived from species compatible with the host cell are used in connection with these hosts. Both expression and cloning vectors contain a nucleic acid sequence that enables the vector to replicate in one or more selected host cells, as well as marking sequences which are capable of providing phenotypic selection in transformed cells. Such sequences are well known for a variety of bacteria, yeast, and viruses. The origin of replication from the plasmid pBR322, which contains genes encoding ampicillin (Amp) and tetracycline (Tet) resistance and thus provides easy means for identifying transformed cells, is suitable for most Gram-negative bacteria, the 2 plasmid origin is suitable for yeast, and various viral origins (SV40, polyoma, adenovirus, VSV or BPV) are useful for cloning vectors in mammalian cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as λGEM™-11 may be utilized in making a recombinant vector which can be used to transform susceptible host cells such as E. coli LE392.

The expression vector of the invention may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g., the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters recognized by a variety of potential host cells are well known. Promoters suitable for use with prokaryotic hosts include the PhoA promoter, the β-galactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776) and hybrid promoters such as the tac (deBoer et al., Proc. Natl. Acad. Sci. USA, 80:21-25 (1983)) or the tac promoter. Promoters for use in bacterial systems also will contain a Shine-Dalgarno (S.D.) sequence operably linked to the DNA encoding anti-CTHRC1 antibody. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB-strains) provide cytoplasm conditions that are favorable for disulfide bond formation, thereby permitting proper folding and assembly of expressed protein subunits. Proba and Pluckthun Gene, 159:203 (1995).

The present invention provides an expression system in which the quantitative ratio of expressed polypeptide components can be modulated in order to maximize the yield of secreted and properly assembled fusion proteins of the invention. Such modulation is accomplished at least in part by simultaneously modulating translational strengths for the polypeptide components.

One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence, although silent changes in the nucleotide sequence are preferred. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A Companion to Methods in Enzymol. 4:151-158.

Preferably, a set of vectors is generated with a range of TIR strengths for each cistron therein. This limited set provides a comparison of expression levels of each chain as well as the yield of the desired antibody products under various TIR strength combinations. TIR strengths can be determined by quantifying the expression level of a reporter gene as described in detail in Simmons et al. U.S. Pat. No. 5,840,523. Based on the translational strength comparison, the desired individual TIRs are selected to be combined in the expression vector constructs of the invention.

Suitable prokaryotes include but are not limited to archaebacteria and eubacteria, such as Gram-negative or Gram-positive organisms, for example, Enterobacteriaceae such as E. coli. Various E. coli strains are publicly available, such as K12 strain MM294 (ATCC 31,446); X1776 (ATCC 31,537); W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, Rhizobia, Vitreoscilla, Paracoccus and Streptomyces. These examples are illustrative rather than limiting. E. coli strain W3110 is one particularly preferred host or parent host because it is a common host strain for recombinant DNA product fermentations. Preferably, the host cell secretes minimal amounts of proteolytic enzymes. For example, strain W3110 (Bachmann, Cellular and Molecular Biology, vol. 2 (Washington, D.C.: American Society for Microbiology, 1987), pp. 1190-1219; ATCC Deposit No. 27,325) may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac) 169 degP ompT kanr; E. coli W3110 strain 37D6, which has the complete genotype tonA ptr3 phoA E15 (argF-lac) 169 degP ompT rbs7 ilvG kanr; E. coli W3110 strain 40B4, which is strain 37D6 with a non-kanamycin resistant degP deletion mutation; E. coli W3110 strain 33D3 having genotype W3110 ΔfhuA (ΔtonA) ptr3 lac Iq lacL8 ΔompTΔ(nmpc-fepE) degP41 kanR (U.S. Pat. No. 5,639,635) and an E. coli strain having mutant periplasmic protease disclosed in U.S. Pat. No. 4,946,783 issued 7 Aug. 1990. Other strains and derivatives thereof, such as E. coli 294 (ATCC 31,446), E. coli B, E. coli λ 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture. Alternatively, in vitro methods of cloning, e.g., PCR or other nucleic acid polymerase reactions, are suitable.

Antibody fusion proteins can be produced in bacteria, in particular when glycosylation and Fc effector function are not needed. Full length antibodies have greater half life in circulation. Production in E. coli is faster and more cost efficient. For expression of antibody fragments and polypeptides in bacteria, see, e.g., U.S. Pat. Nos. 5,648,237; 5,789,199 and 5,840,523, which describe translation initiation region (TIR) and signal sequences for optimizing expression and secretion, these patents incorporated herein by reference. After expression, the fusion protein is isolated from the E. coli cell paste in a soluble fraction and can be purified through, e.g., a protein A or G column depending on the isotype. Final purification can be carried out similar to the process for purifying antibody expressed e.g., in CHO cells.

### Eukaryotic Host Cells

In addition to prokaryotes, eukaryotic microbes such as filamentous fungi or yeast are suitable cloning or expression hosts for anti-CTHRC1 fusion protein-encoding vectors. Saccharomyces cerevisiae is a commonly used lower eukaryotic host microorganism. Others include Schizosaccharomyces pombe (Beach and Nurse, Nature, 290: 140 (1981); EP 139,383 published 2 May 1985); Kluyveromyces hosts (U.S. Pat. No. 4,943,529; Fleer et al., Bio/Technology, 9: 968-75 (1991)) such as, e.g., K. lactis (MW98-8C, CBS683, CBS4574; Louvencourt et al., J. Bacteriol., 154(2):737-742 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 (1988)); Candida; Trichoderma reesia (EP 244,234); Neurospora crassa (Case et al., Proc. Natl. Acad. Sci. USA, 76:5259-5263 (1979)); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 (1983); Tilburn et al., Gene, 26:205-221 (1983); Yelton et al., Proc. Natl. Acad. Sci. USA, 81: 1470-1474 (1984)) and A. niger (Kelly and Hynes, EMBO J., 4:475-479 (1985)). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated fusion protein are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

The vector components generally include, but are not limited to, one or more of the following: a signal sequence, an origin of replication, one or more marker genes, an enhancer element, a promoter, and a transcription termination sequence.

### Signal Sequence Component

A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the fusion protein.

### Origin of Replication

Generally, an origin of replication component is not needed for mammalian expression vectors. For example, the SV40 origin may typically be used only because it contains the early promoter.

### Selection Gene Component

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

An example of suitable selectable markers for mammalian cells are those that enable the identification of cells competent to take up the anti-CTHRC1 fusion protein-encoding nucleic acid, such as DHFR or thymidine kinase, metallothionein-I and -II, preferably primate metallothionein genes, adenosine deaminase, ornithine decarboxylase, etc. An appropriate host cell when wild-type DHFR is employed is the CHO cell line deficient in DHFR activity (e.g., ATCC CRL-9096), prepared and propagated as described by Urlaub et al., Proc. Natl. Acad. Sci. USA, 77:4216 (1980). For example, cells transformed with the DHFR selection gene are first identified by culturing all of the transformants in a culture medium that contains methotrexate (Mtx), a competitive antagonist of DHFR. Alternatively, host cells (particularly wild-type hosts that contain endogenous DHFR) transformed or co-transformed with DNA sequences encoding a anti-CTHRC1 fusion protein, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979); Kingsman et al., Gene, 7:141 (1979); Tschemper et al., Gene, 10:157 (1980)). The trp1 gene provides a selection marker for a mutant strain of yeast lacking the ability to grow in tryptophan, for example, ATCC No. 44076 or PEP4-1 (Jones, Genetics, 85:12 (1977)).

### Promoter Component

Expression and cloning vectors usually contain a promoter operably linked to the anti-CTHRC1 fusion protein-encoding nucleic acid sequence to direct mRNA synthesis. Promoters recognized by a variety of potential host cells are well known.

Virtually alleukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase (Hitzeman et al., J. Biol. Chem., 255:2073 (1980)) or other glycolytic enzymes (Hess et al., J. Adv. Enzyme Reg., 7:149 (1968); Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Anti-CTHRC1 fusion protein transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

### Enhancer Element Component

Transcription of a DNA encoding the anti-CTHRC1 fusion protein by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the anti-CTHRC1 fusion protein coding sequence, but is preferably located at a site 5′ from the promoter.

### Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding anti-CTHRC1 fusion protein. One useful transcription termination component is the bovine growth hormone polyadenylation region. See WO94/11026 and the expression vector disclosed therein.

Still other methods, vectors, and host cells suitable for adaptation to the synthesis of anti-CTHRC1 fusion protein in recombinant vertebrate cell culture are described in Gething et al., Nature, 293:620-625 (1981); Mantei et al., Nature, 281:40-46 (1979); EP 117,060; and EP 117,058.

Host cells are transformed with the above-described expression or cloning vectors for anti-CTHRC1 fusion protein production and cultured in conventional nutrient media modified as appropriate for inducing promoters, selecting transformants, or amplifying the genes encoding the desired sequences.

### Culturing the Host Cells

The host cells used to produce the anti-CTHRC1 fusion protein of this invention may be cultured in a variety of media.

### Prokaryotic Host Cells

Prokaryotic cells used to produce the polypeptides of the invention are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the invention, protein expression is induced under conditions suitable for the activation of the promoter. In one aspect of the invention, PhoA promoters are used for controlling transcription of the polypeptides. Accordingly, the transformed host cells are cultured in a phosphate-limiting medium for induction. In some embodiments, the phosphate-limiting medium is the C.R.A.P medium (see, e.g., Simmons et al., J. Immunol. Methods (2002), 263: 133-47). A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In one aspect of the invention, fusion protein production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted fusion protein polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274: 19601-5; U.S. Pat. Nos. 6,083,715; 6,027,888; Bothmann and Pluckthun (2000) J. Biol. Chem. 275:17100-5; Ramm and Pluckthun (2000) J. Biol. Chem. 275:17106-13; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; U.S. Pat. Nos. 5,264,365; 5,508,192; Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the invention.

### Eukaryotic Host Cells

Commercially available media such as Ham's F10 (Sigma), Minimal Essential Medium ((MEM), (Sigma), RPMI-1640 (Sigma), and Dulbecco's Modified Eagle Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Detecting Gene Amplification Expression

Gene amplification and/or expression may be measured in a sample directly, for example, by conventional Southern blotting, Northern blotting to quantitate the transcription of mRNA (Thomas, Proc. Natl. Acad. Sci. USA, 77: 5201-5 (1980)), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence CTHRC1 polypeptide or against a synthetic peptide based on the DNA sequences provided herein or against exogenous sequence fused to CTHRC1 DNA and encoding a specific antibody epitope.

### Purification of Anti-CTHRC1 Fusion Protein

Forms of anti-CTHRC1 fusion protein may be recovered from culture medium or from host cell lysates. If membrane-bound, it can be released from the membrane using a suitable detergent solution (e.g., Triton-X 100) or by enzymatic cleavage. Cells employed in expression of anti-CTHRC1 fusion protein can be disrupted by various physical or chemical means, such as freeze-thaw cycling, sonication, mechanical disruption, or cell lysing agents.

It may be desired to purify anti-CTHRC1 fusion protein from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the anti-CTHRC1 fusion protein. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular anti-CTHRC1 fusion protein produced.

When using recombinant techniques, the fusion protein can be produced intracellularly, in the periplasmic space, or directly secreted into the medium. If the fusion protein is produced intracellularly, as a first step, the particulate debris, either host cells or lysed fragments, are removed, for example, by centrifugation or ultrafiltration. Carter et al., Bio/Technology 10: 163-7 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the fusion protein is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The fusion protein composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the fusion protein. Protein A can be used to purify antibodies that are based on human γ1, γ2 or γ4 heavy chains (Lindmark et al., J. Immunol. Meth. 62: 1-13 (1983)). Protein G is recommended for all mouse isotypes and for human 73 (Guss et al., EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the CTHRC1 binding moiety comprises a CH3 domain, the Bakerbond ABX™ resin (J. T. Baker, Phillipsburg, NJ) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the fusion protein to be recovered.

Following any preliminary purification step(s), the mixture comprising the fusion protein of interest and contaminants may be subjected to low pH hydrophobic interaction chromatography using an elution buffer at a pH between about 2.5-4.5, and generally at low salt concentrations (e.g., from about 0-0.25M salt).

### Assays

### Activity Assays

In one aspect, assays are provided for identifying anti-CTHRC1 antibodies thereof having biological activity. Biological activity may include, e.g., the ability to inhibit cell growth or proliferation (e.g., “cell killing” activity), or the ability to induce cell death, including programmed cell death (apoptosis). Antibodies having such biological activity in vivo and/or in vitro are also provided.

In certain embodiments, an anti-CTHRC1 antibody is tested for its ability to inhibit cell growth or proliferation in vitro. Assays for inhibition of cell growth or proliferation are well known in the art. Certain assays for cell proliferation, exemplified by the “cell killing” assays described herein, measure cell viability. One such assay is the CellTiter-Glo™ Luminescent Cell Viability Assay, which is commercially available from Promega (Madison, WI). That assay determines the number of viable cells in culture based on quantitation of ATP present, which is an indication of metabolically active cells. See Crouch et al (1993) J. Immunol. Meth. 160: 81-8, U.S. Pat. No. 6,602,677. The assay may be conducted in 96- or 384-well format, making it amenable to automated high-throughput screening (HTS) (see Cree et al (1995) AntiCancer Drugs 6: 398-404). The assay procedure involves adding a single reagent (CellTiter-Glo® Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera imaging device. The luminescence output is expressed as relative light units (RLU).

Another assay for cell proliferation is the “MTT” assay, a colorimetric assay that measures the oxidation of 3-(4,5-dimethylthiazol-2-yl)-2,5-diphenyltetrazolium bromide to formazan by mitochondrial reductase. Like the CellTiter-Glo™ assay, this assay indicates the number of metabolically active cells present in a cell culture (see, e.g., Mosmann (1983) J. Immunol. Meth. 65:55-63, and Zhang et al. (2005) Cancer Res. 65: 3877-82).

In one aspect, an anti-CTHRC1 antibody is tested for its ability to induce cell death in vitro. Assays for induction of cell death are well known in the art. In some embodiments, such assays measure, e.g., loss of membrane integrity as indicated by uptake of propidium iodide (PI), trypan blue (see Moore et al. Cytotechnology, 17: 1-11 (1995)), or 7AAD. In an exemplary PI uptake assay, cells are cultured in Dulbecco's Modified Eagle Medium (D-MEM):Ham's F-12 (50:50) supplemented with 10% heat-inactivated FBS (Hyclone) and 2 mM L-glutamine. Thus, the assay is performed in the absence of complement and immune effector cells. Cells are seeded at a density of 3×106 per dish in 100×20 mm dishes and allowed to attach overnight. The medium is removed and replaced with fresh medium alone or medium containing various concentrations of the antibody. The cells are incubated for a 3-day time period. Following treatment, monolayers are washed with PBS and detached by trypsinization. Cells are then centrifuged at 1200 rpm for 5 minutes at 4° C., the pellet resuspended in 3 mL cold Ca2+ binding buffer (10 mM Hepes, pH 7.4, 140 mM NaCl, 2.5 mM CaCl₂)) and aliquoted into 35 mm strainer-capped 12×75 mm tubes (1 mL per tube, 3 tubes per treatment group) for removal of cell clumps. Tubes then receive PI (10 g/mL). Samples are analyzed using a FACSCAN™ flow cytometer and FACSCONVERT™ CellQuest software (Becton Dickinson). Antibodies which induce statistically significant levels of cell death as determined by PI uptake are thus identified.

In one aspect, an anti-CTHRC1 antibody is tested for its ability to induce apoptosis (programmed cell death) in vitro. An exemplary assay for antibodies that induce apoptosis is an annexin binding assay. In an exemplary annexin binding assay, cells are cultured and seeded in dishes as discussed in the preceding paragraph. The medium is removed and replaced with fresh medium alone or medium containing 0.001 to 10 μg/mL of the antibody. Following a three-day incubation period, monolayers are washed with PBS and detached by trypsinization. Cells are then centrifuged, resuspended in Ca2+ binding buffer, and aliquoted into tubes as discussed in the preceding paragraph. Tubes then receive labeled annexin (e.g., annexin V-FITC) (1 μg/mL). Samples are analyzed using a FACSCAN™ flow cytometer and FACSCONVERT™ CellQuest software (BD Biosciences). Antibodies that induce statistically significant levels of annexin binding relative to control are thus identified. Another exemplary assay for antibodies that induce apoptosis is a histone DNA ELISA colorimetric assay for detecting internucleosomal degradation of genomic DNA. Such an assay can be performed using, e.g., the Cell Death Detection ELISA kit (Roche, Palo Alto, CA).

Cells for use in any of the above in vitro assays include cells or cell lines that naturally express CTHRC1 or that have been engineered to express CTHRC1. Such cells include tumor cells that overexpress CTHRC1 relative to normal cells of the same tissue origin. Such cells also include cell lines (including tumor cell lines) that express CTHRC1 and cell lines that do not normally express CTHRC1 but have been transfected with nucleic acid encoding CTHRC1.

In one aspect, an anti-CTHRC1 antibody thereof is tested for its ability to inhibit cell growth or proliferation in vivo. In certain embodiments, an anti-CTHRC1 antibody thereof is tested for its ability to inhibit tumor growth in vivo. In vivo model systems, such as xenograft models, can be used for such testing. In an exemplary xenograft system, human tumor cells are introduced into a suitably immunocompromised non-human animal, e.g., a SCID mouse. An antibody of the invention is administered to the animal. The ability of the antibody to inhibit or decrease tumor growth is measured. In certain embodiments of the above xenograft system, the human tumor cells are tumor cells from a human patient. In certain embodiments, the human tumor cells are introduced into a suitably immunocompromised non-human animal by subcutaneous injection or by transplantation into a suitable site, such as a mammary fat pad.

### Binding Assays and Other Assays

In one aspect, an anti-CTHRC1 antibody is tested for its antigen binding activity. For example, in certain embodiments, an anti-CTHRC1 antibody is tested for its ability to bind to CTHRC1 expressed on the surface of a cell. A FACS assay may be used for such testing.

In one aspect, competition assays may be used to identify a monoclonal antibody that competes with a monoclonal antibody comprising the variable domains of any one of SEQ ID NOs: 75-84 or a chimeric antibody comprising the variable domain of the monoclonal antibody comprising the sequences of Table 2 and Table 3, and constant domains from IgG1 or IgG4 for binding to CTHRC1. In certain embodiments, such a competing antibody binds to the same epitope (e.g., a linear or a conformational epitope) that is bound by a monoclonal antibody comprising the variable domains of any one of SEQ ID NOs: 75-84 or a chimeric antibody comprising the variable domain of the monoclonal antibody comprising the sequences of Table 2 and Table 3, and constant domains from IgG1 or IgG4. Exemplary competition assays include, but are not limited to, routine assays such as those provided in Harlow and Lane (1988) Antibodies: A Laboratory Manual ch. 14 (Cold Spring Harbor Laboratory, Cold Spring Harbor, NY). Detailed exemplary methods for mapping an epitope to which an antibody binds are provided in Morris (1996) “Epitope Mapping Protocols,” in Methods in Molecular Biology vol. 66 (Humana Press, Totowa, NJ). Two antibodies are said to bind to the same epitope if each blocks binding of the other by 50% or more.

In one aspect, purified anti-CTHRC1 antibodies can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

### Methods of Treatment

A anti-CTHRC1 fusion protein of the invention may be used in, for example, in vitro, ex vivo, and in vivo therapeutic methods. In one aspect, the invention provides methods for inhibiting cell growth or proliferation, either in vivo or in vitro, the method comprising exposing a cell to anti-CTHRC1 fusion protein or composition thereof of the present disclosure under conditions permissive for binding of the fusion protein to CTHRC1. “Inhibiting cell growth or proliferation” means decreasing a cell's growth or proliferation by at least 10%, 20%, 30%, 40%, 50%, 60%, 70%, 80%, 90%, 95%, or 100%, and includes inducing cell death. In certain embodiments, the cell is a tumor cell. The anti-CTHRC1 fusion protein or composition thereof may additionally or alternatively (i) inhibit tumor metastasis in vivo; (ii) inhibit tumor growth in vivo; (iii) decrease tumor size in vivo; (iv) inhibit tumor vascularization in vivo; (v) exhibit cycotoxic activity activity on tumor cells and cancer associated fibroblasts expressing and/or displaying CTHRC1 in vivo; (vi) exhibit cytostatic activity on tumor cells or cancer associated fibroblasts expressing and/or displaying CTHRC1 in vivo; (vii) enhance infiltration of anti-tumor immune cells in vivo; or (viii) prevent suppression of immune-cells in the tumor microenvironment in vivo.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the fusion proteins of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. In certain embodiments, CAR T cells can be used therapeutically for patients suffering from non-hematological tumors such as solid tumors arising from breast, CNS, and skin malignancies.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile ductcarcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In some embodiments, a method of treating a cell proliferative disorder can include a step of administering to a subject a therapeutically effective amount of a anti-CTHRC1 fusion protein or pharmaceutical composition thereof of any of the foregoing embodiments. In certain embodiments, the cell proliferative disorder is associated with increased expression, display and/or activity of CTHRC1. For example, in certain embodiments, the cell proliferative disorder is associated with increased expression and/or display of CTHRC1 on the surface of a cell, either directly or in a complex. In certain embodiments, the cell proliferative disorder is a tumor or a cancer. In certain embodiments, the fusion protein can be administered at a dose of 0.001 mg/kg to about 100 mg/kg based on the patient's body weight.

In some embodiments, a method of treating cancer can include a step of administering to a subject a therapeutically effective amount of an anti-CTHRC1 fusion protein or pharmaceutical composition thereof of any of the foregoing embodiments. By way of example, but not limitation, the cancer can be selected from the group consisting of breast cancer, lung cancer, colon cancer, ovarian cancer, melanoma cancer, bladder cancer, renal cancer, kidney cancer, liver cancer, head and neck cancer, colorectal cancer, melanoma, pancreatic cancer, gastric carcinoma cancer, esophageal cancer, mesothelioma, prostate cancer, leukemia, lymphomas, and myelomas. In certain embodiments, the cell proliferative disorder is a tumor or a cancer. In certain embodiments, the fusion protein can be administered at a dose of 0.001 mg/kg to about 100 mg/kg based on the patient's body weight.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject).

In any of the foregoing embodiments, the step of administering the anti-CTHRC1 fusion protein or pharmaceutical composition thereof (and any additional therapeutic agent or adjuvant) can be performed by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. By way of further example, but not limitation, the administration can be performed by known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. In addition, the anti-CTHRC1 fusion protein is suitably administered by pulse infusion, particularly with declining doses of the anti-CTHRC1 fusion protein. Dosing can be by any suitable route, e.g., by injections, such as intravenous or subcutaneous injections, depending in part on whether the administration is brief or chronic. In some embodiments, intravenous or subcutaneous administration of the anti-CTHRC1 fusion protein is preferred.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally.

Anti-CTHRC1 fusion proteins of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

As discussed, the anti-CTHRC1 fusion proteins are administered to a human patient, in accordance with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. In some embodiments, intravenous or subcutaneous administration of the anti-CTHRC1 fusion protein is preferred.

The anti-CTHRC1 fusion protein composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The CTHRC1 binding moieties of the invention can be in the different forms encompassed by the definition of “antibody” herein. Thus, the antibodies include full length or intact antibody, antibody fragments, native sequence antibody or amino acid variants, humanized, chimeric or fusion antibodies, and functional fragments thereof. In fusion antibodies an antibody sequence is fused to a heterologous polypeptide sequence. The antibodies can be modified in the Fc region to provide desired effector functions. As discussed in more detail in the sections herein, with the appropriate Fc regions, the naked antibody bound on the cell surface can induce cytotoxicity, e.g., via antibody-dependent cellular cytotoxicity (ADCC) or by recruiting complement in complement dependent cytotoxicity, or some other mechanism. Alternatively, where it is desirable to eliminate or reduce effector function, so as to minimize side effects or therapeutic complications, certain other Fc regions may be used.

In one embodiment, the antibody (i) competes for binding to the same epitope, and/or (ii) binds substantially to the same epitope, as the antibodies of the invention.

Methods of producing the above fusion proteins are described in detail herein.

The present anti-CTHRC1 fusion proteins are useful for treating a CTHRC1-displaying cancer or alleviating one or more symptoms of the cancer in a mammal. The cancers encompass metastatic cancers of any of the cancers described herein. The fusion protein is able to bind to at least a portion of the cancer cells that display CTHRC1 directly or in a complex in the mammal. In a preferred embodiment, the fusion protein is effective to destroy or kill CTHRC1-displaying tumor cells or inhibit the growth of such tumor cells, in vitro or in vivo, upon binding to CTHRC1 epitope on the cell. In other preferred embodiments, the fusion proteins are effective to i) inhibit tumor metastasis in vivo; (ii) inhibit tumor growth in vivo; (iii) decrease tumor size in vivo; (iv) inhibit tumor vascularization in vivo; (v) exhibit cytotoxic activity on tumor cells and cancer associated fibroblasts expressing and/or displaying CTHRC1 in vivo; (vi) exhibit cytostatic activity on a tumor cells or cancer associated fibroblasts expressing and/or displaying CTHRC1 in vivo; (vii) enhance infiltration of anti-tumor immune cells in vivo; or (viii) prevent suppression of immune-cells in the tumor microenvironment in vivo.

The invention provides a composition comprising an anti-CTHRC1 fusion protein of the invention, and a carrier. The invention also provides formulations comprising an anti-CTHRC1 fusion protein of the invention, and a carrier. In one embodiment, the formulation is a therapeutic formulation comprising a pharmaceutically acceptable carrier.

Another aspect of the invention is isolated nucleic acids encoding the anti-CTHRC1 fusion proteins. For example, nucleic acids encoding both the H and L chains and especially the hypervariable region residues, chains which encode the native sequence CTHRC1 binding moiety as well as variants, modifications and humanized versions of the antibody, are encompassed.

The invention also provides methods useful for treating a CTHRC1 polypeptide-displaying cancer or alleviating one or more symptoms of the cancer in a mammal, comprising administering a therapeutically effective amount of an anti-CTHRC1 fusion protein to the mammal. The antibody therapeutic compositions can be administered short term (acute) or chronic, or intermittent as directed by physician. Also provided are methods of inhibiting the growth of, and killing a CTHRC1 polypeptide-displaying cell.

For the prevention or treatment of disease, the dosage and mode of administration will be chosen by the physician according to known criteria. The appropriate dosage of anti-CTHRC1 fusion protein will depend on the type of disease to be treated, as defined above, the severity and course of the disease, whether the anti-CTHRC1 fusion protein is administered for preventive or therapeutic purposes, previous therapy, the patient's clinical history and response to the antibody, and the discretion of the attending physician. The anti-CTHRC1 fusion protein is suitably administered to the patient at one time or over a series of treatments. Preferably, the anti-CTHRC1 fusion protein or pharmaceutical composition thereof is administered by intravenous infusion or by subcutaneous injections. Depending on the type and severity of the disease, about 1 μg/kg to about 100 mg/kg body weight (e.g., about 0.1-30 mg/kg/dose) of anti-CTHRC1 fusion protein can be an initial candidate dosage for administration to the patient, whether, for example, by one or more separate administrations, or by continuous infusion. A dosing regimen can comprise administering an initial loading dose of about 4 mg/kg, followed by a weekly maintenance dose of about 2 mg/kg of the anti-CTHRC1 fusion protein. However, other dosage regimens may be useful. A typical daily dosage might range from about 1 μg/kg to 1000 mg/kg or more, depending on the factors mentioned above. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

In any of the foregoing embodiments, the method of treatment can further include administering an allogenic or autologous CAR-T or NK cell therapy to the subject.

In any of the foregoing embodiments, the subject can be a human or a non-human mammal.

In certain aspects, a pharmaceutical composition of the present disclosure can be used in the preparation of a medicament for the treatment of a cell proliferative disorder, preferably cancer.

### Articles of Manufacture and Kits

Another embodiment of the invention is an article of manufacture containing materials useful for the treatment, prevention and/or diagnosis of CTHRC1-displaying cancer. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating, preventing and/or diagnosing the cancer condition and may have a sterile access port (for example the container may be an intravenous solution bag or a vial having a stopper pierceable by a hypodermic injection needle). At least one active agent in the composition is an anti-CTHRC1 antibody of the invention, or a CAR-modified immune cell of the invention, or a nucleic acid of the invention. Optionally, a composition further comprises a carrier, for example a pharmaceutically acceptable carrier. The label or package insert indicates that the composition is used for treating cancer. The label or package insert will further comprise instructions for administering the antibody composition to the cancer patient. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for CTHRC1-displaying cell killing assays, for purification or immunoprecipitation of CTHRC1 polypeptide from cells. For isolation and purification of CTHRC1 polypeptide, the kit can contain an anti-CTHRC1 antibody coupled to beads (e.g., sepharose beads). Kits can be provided which contain the antibodies for detection and quantitation of CTHRC1 polypeptide in vitro, e.g., in an ELISA or a Western blot. As with the article of manufacture, the kit comprises a container and a label or package insert on or associated with the container. The container holds a composition comprising at least one anti-CTHRC1 antibody of the invention. Additional containers may be included that contain, e.g., diluents and buffers, control antibodies. The label or package insert may provide a description of the composition as well as instructions for the intended in vitro or detection use.

For example, a kit can comprise a first container comprising a composition comprising one or more CTHRC1 antibodies or CAR modified immune cells, such as CAR-T or CAR-NK cells, or CAR macrophages, of the invention; and a second container comprising a buffer. The buffer may be pharmaceutically acceptable.

The following examples are offered for illustrative purposes only and are not intended to limit the scope of the present invention in any way.

All patent, patent application, and literature references cited in the present specification are hereby incorporated by reference in their entirety.

### EXAMPLES

### Example 1: CTHRC1 Expression on Tumor and Stroma in Human Colorectal Cancer Samples by IHC

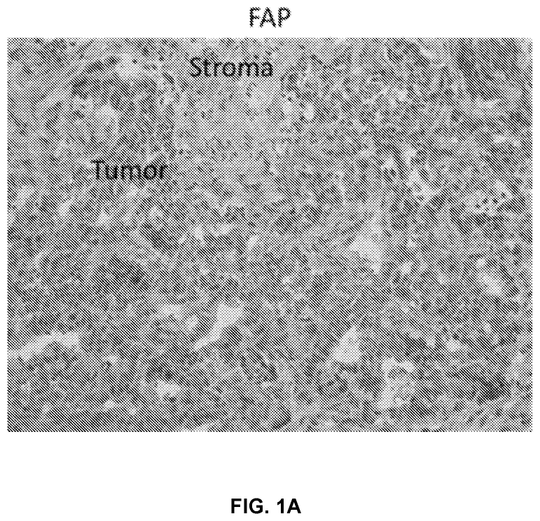

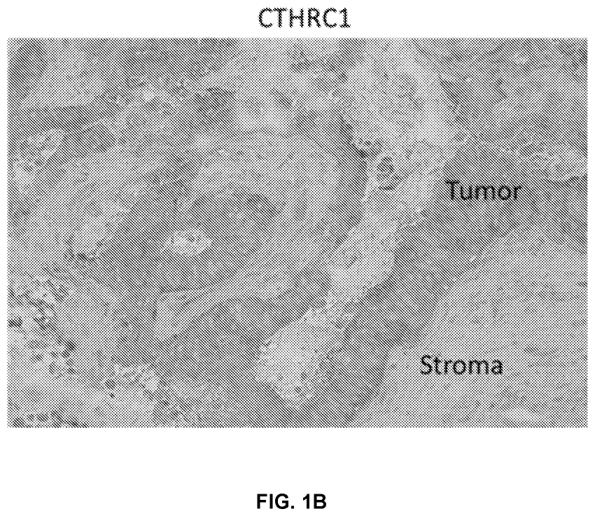

A tissue sample from a colorectal cancer patient was stained with FAP as shown in FIG. 1A. A separate tissue sample for a colorectal cancer patient was assessed by IHC using an anti-CTHRC1, rabbit polyclonal antibody (Abcam, ab 85739) as shown in FIG. 1B. Tissue slides from a subject with stage IV colorectal cancer were obtained from commercial sources and stained with the indicated antibody at the recommended dilution for immunohistochemistry. After incubation with primary antibody, slides were washed and bound mAb detected by common chromogenic methods. As shown in FIG. 1A, tumor stroma is stained with FAP while tumor nests (blue) are devoid of FAP staining. However, as shown FIG. 1B, CTHRC1 localizes in both tumor stroma and tumor nests which comports with scRNA data (not shown).

### Example 2: Membrane Binding of CTHRC1 Antibodies

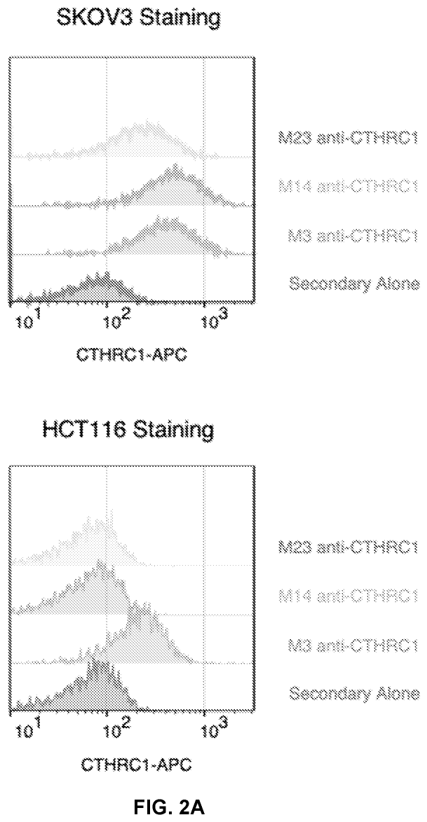

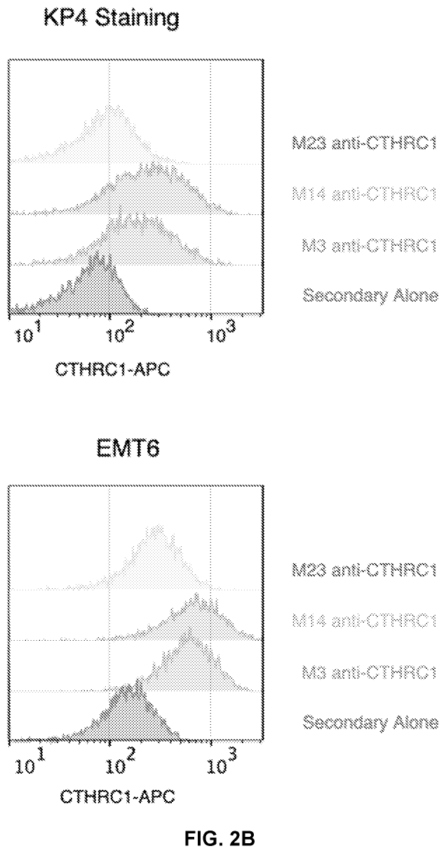

Staining of tumor cell lines by anti-CTHRC1 mAbs was assessed by flow cytometry. Human SKOv3 ovarian cancer, KP4 PDAC, and HCT115 colorectal cancer lines as well as the mouse EMT6 breast cancer line were selected. Cell lines were incubated with 0.25 ug/mL recombinant CTHRC1 to form complexes on the cell surface. Following incubation, cells were washed and anti-CTHRC1 mAbs were added at 10 ug/mL and incubated for 30 minutes. Cells were then washed and incubated with an anti-mouse secondary antibody diluted 1:250 in FACS buffer for 20 minutes. Cells were washed again and resuspended in FACS buffer for analysis on a Sony Cell Analyzer. Results are shown in FIGS. 2A-2D which confirm that anti-CTHRC1 antibodies can bind to the surface of tumor cells.

### Example 3: Induction of T-Cell Proliferation by CTHRC1 scFv 4-1BBL Constructs

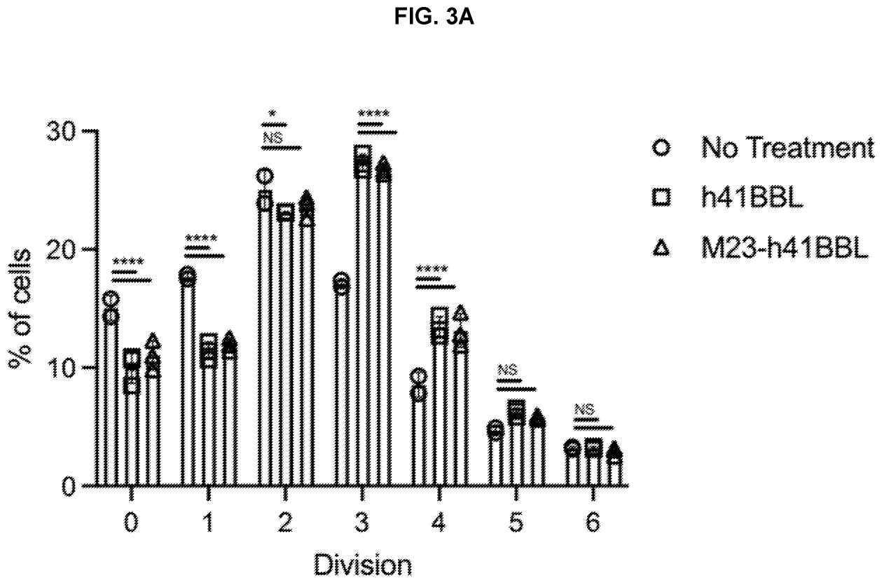

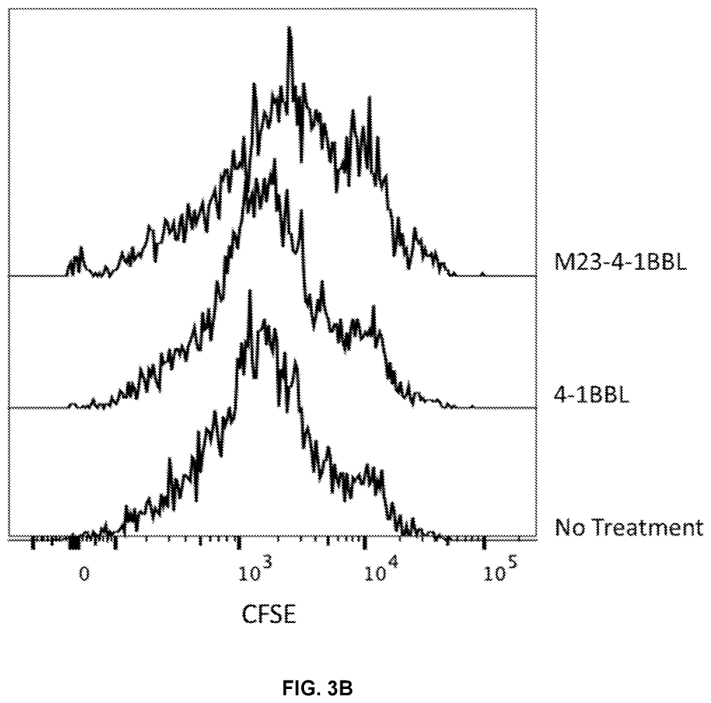

Jurkat T cells were labeled with CFSE, washed, and stimulated with 0.1 ug/mL anti-CD3+5 ug/mL anti-CD28 alone or with recombinant 4-1BBL or M23-4-1BBL at 0.5 ug/mL (3-fold molar excess of ligand vs fusion protein) for 72 hours. Stimuli were removed and cells allowed to remain in culture for an additional 24 hours. CFSE dilution was assessed by flow cytometry on a BD LSR Fortessa X20. Index of proliferation was assessed as CFSE dilution series and degree of proliferation was deemed significant by 2-way ANOVA. Results are shown in FIGS. 3A-3B.

### Example 4: In Vivo Efficacy of CTHRC1 scFv 4-1BBL Constructs

8-10 week old female BALB/c mice were inoculated with 1×10⁵ EMT6 cells suspended in Geltrex subcutaneously. Tumor growth was measured, and mice were randomized to treatment when average tumor volume was 120 mm³.

M23-41BBL (SEQ ID NO: 101), a fusion protein of an anti-CTHRC1 scFv and 4-1BBL, produced recombinantly in HEK cells and purified via binding of a C-terminal His tag using nickel-NTA resin, was administered to each mouse at a dose of 1 mg/kg, 2.5 mg/kg or 5 mg/kg. Tumor growth and survival were assessed three times per week. Dengue-4-1BBL was used as a control.

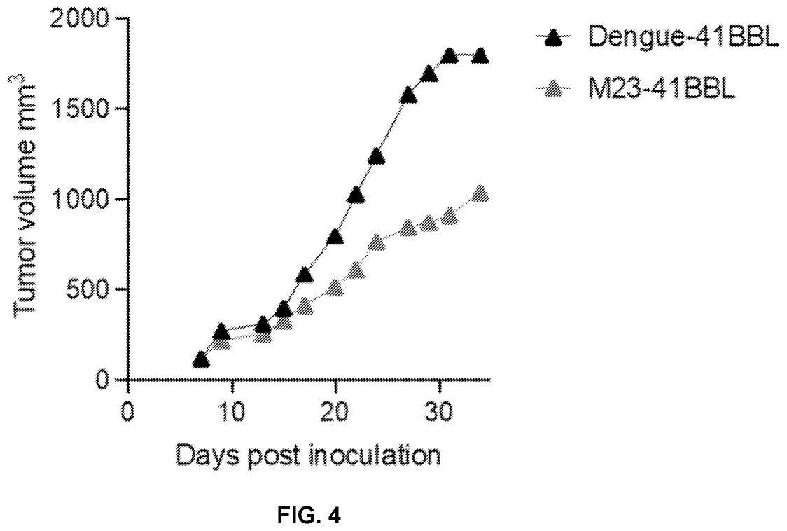

As shown in FIG. 4, tumor volume did not increase as significantly after administration of M23-41BBL versus Dengue-41BBL.

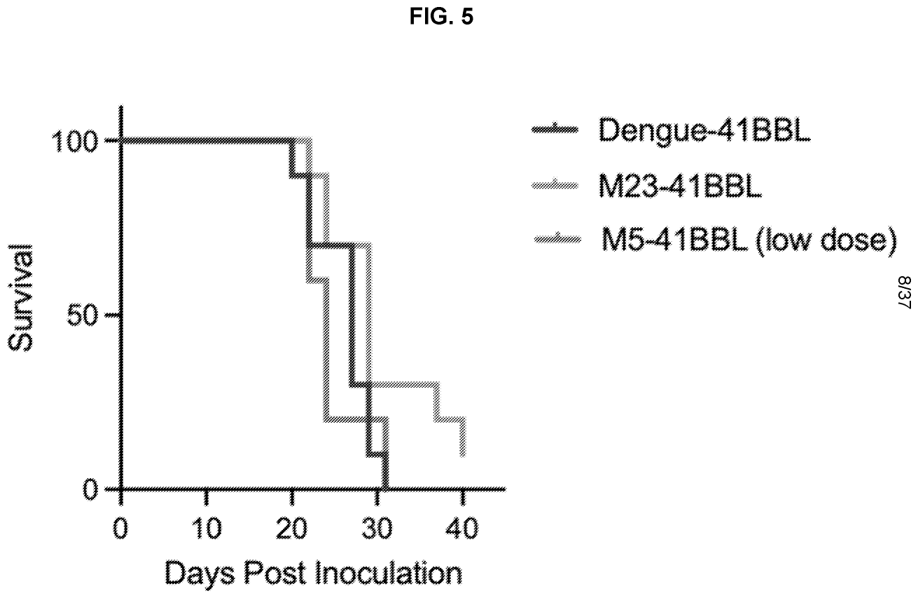

As shown in FIG. 5, mice treated with M23-41BBL had improved survival compared to Dengue-41BBL-treated mice. The M23-41BBL group was dosed at 2.5 mg/kg while M5-41BBL was dosed at 1 mg/kg.

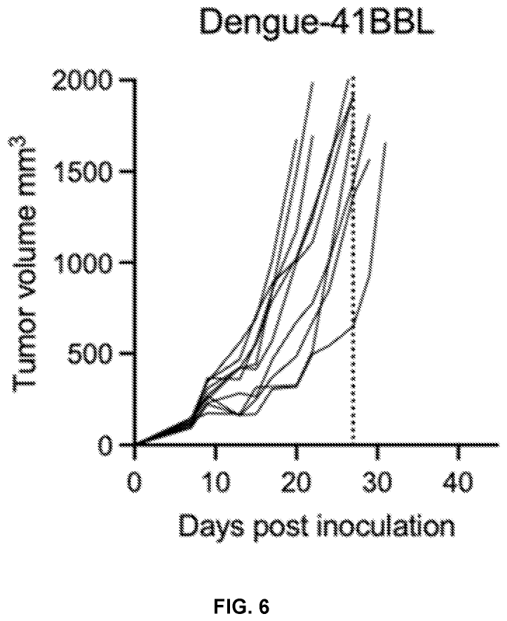

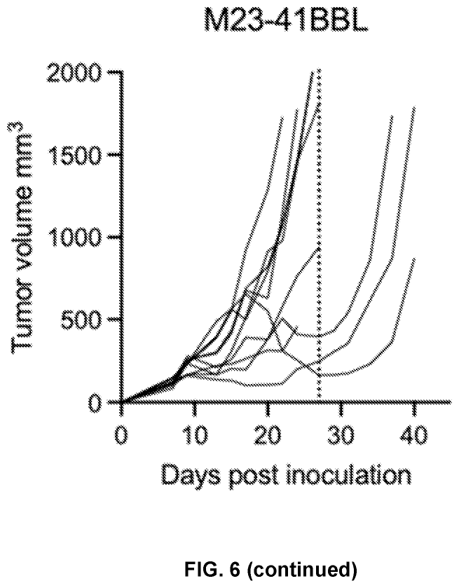

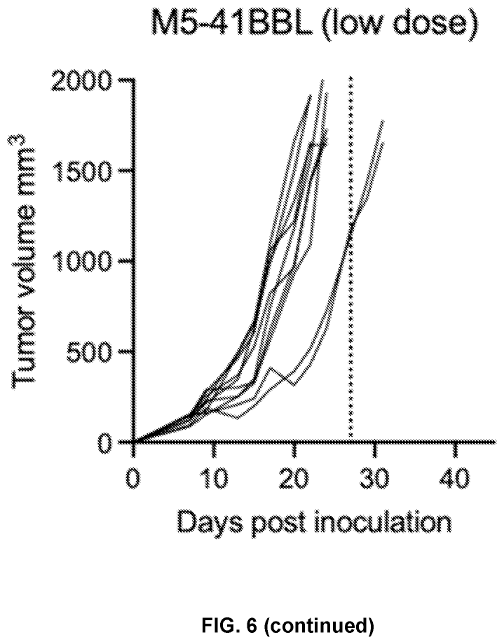

This data is consistent with tumor volume data over time as shown in FIG. 6.

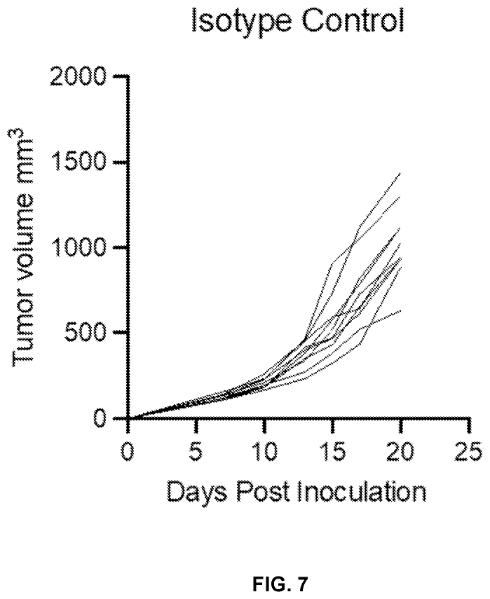

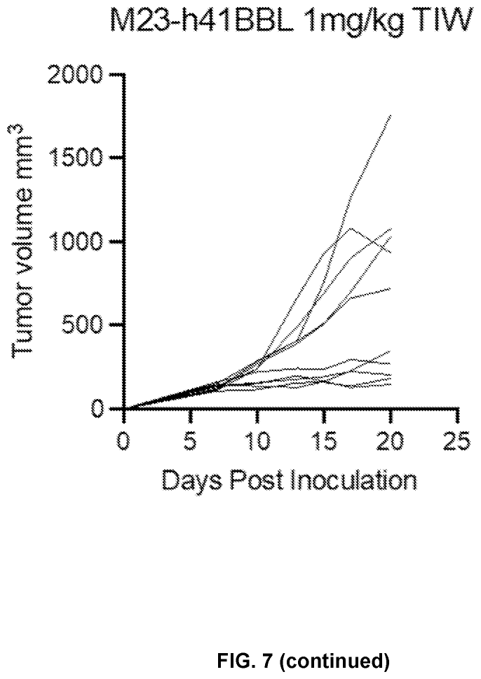

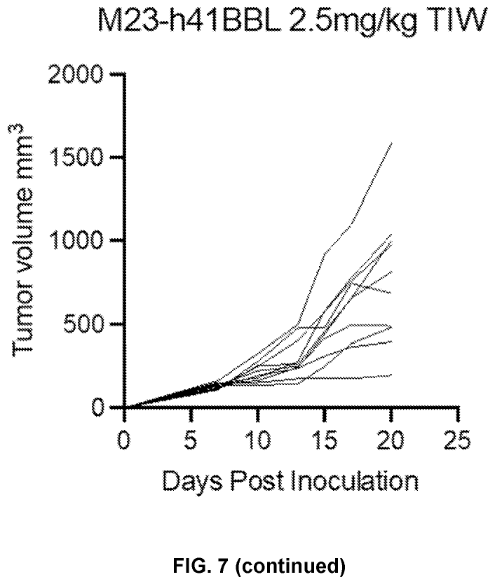

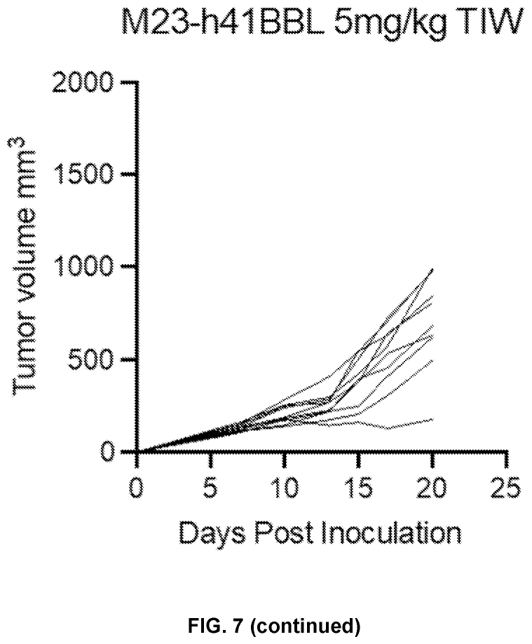

As shown in FIG. 7, low dose (1 mg/kg) treatment with M23-41BBL significantly reduced tumor growth in a subset of mice.

### Example 5: In Vitro T Cell Stimulation Normal PBMCs (Targeted 4-1BBL, OX40L, or GITRL)

CD4 and CD8 T cells are separately isolated from normal healthy donor PBMCs by negative selection using a commercial enrichment kit. Isolated cells are labeled with CFSE and transferred to a tissue culture plate coated with sub-optimal concentration of anti-CD3 antibody+/−CTHRC1-targeted fusion. Following incubation, cell proliferation is assessed by measuring CFSE dilution by Flow Cytometry. Proliferation of both CD4 and CD8 T cells should be induced by OX40L and GITRL fusions. 4-1BBL fusions should predominately stimulate CD8 T cell proliferation and subsets of activated CD4 cells.

### Example 6: In Vitro T and NK Cell Stimulation—Tumor TIL Dissociated Tumor (Targeted 4-1BBL, OX40L, or GITRL)

Dissociated human tumor samples are sourced from a commercial vendor. Cells are incubated in a tissue culture plate+/−CTHRC1-targeted fusion or control. Following incubation, cell proliferation is assessed by measuring Ki-67 expression on individual cell populations by Flow Cytometry. Proliferation of both CD4 and CD8 T cells should be induced by OX40L and GITRL fusions. 4-1BBL fusions should predominately stimulate NK and CD8 T cell proliferation and subsets of activated CD4 cells.

### Example 7: In Vitro NK Cell Stimulation Normal PBMCs (Targeted 4-1BBL)

CD16+NK cells are isolated from normal healthy donor PBMCs by negative selection using a commercial enrichment kit. Isolated cells are labeled with CFSE and transferred to a tissue culture plate+/−CTHRC1-targeted fusion. Following incubation, cell proliferation is assessed by measuring CFSE dilution and upregulation of NK activation markers by Flow Cytometry. Proliferation and upregulation of NK activating receptors should be induced by 4-1BBL fusions.

### Example 8: ADCC Enhancement—Engineered Cell Lines (Targeted 4-1BBL)

CD16+NK cells from normal healthy donors are isolated from PBMCs by negative selection using commercial enrichment kits. NK cells are then plated with target (Her2, CD20, EGFR, etc) expressing tumor cells at various E:T ratios (50:1, 20:1, 10:1, and 5:1) and incubated with a dilution series of target-specific monoclonal antibody capable of effecting ADCC (trastuzumab, rituximab, cetuximab, etc)+/−targeted 4-1BBL. ADCC should be enhanced with the addition of 4-1BBL mediated costimulation.

### Example 9: ADCC Enhancement Primary Tumor and Effector Cells (DTCs and Matched PBMCs) (Targeted 4-1BBL)

Autologous CD16+NK cells from cancer patients are isolated from PBMCs by negative selection using commercial enrichment kits. NK cells are then plated with autologous dissociated tumor cells at various E:T ratios (50:1, 20:1, 10:1, and 5:1) and incubated with a dilution series of target-specific monoclonal antibody capable of effecting ADCC (trastuzumab, rituximab, cetuximab, etc)+/−targeted 4-1BBL. ADCC should be enhanced with the addition of 4-1BBL mediated costimulation.

### Example 10: B Cell Activation and Proliferation In Vitro—Cell Lines Normal B Cells (Targeted CD40L)

B cells are isolated from healthy donor PBMCs by negative selection using a commercially available enrichment kit. Isolated B cells are stimulated with CD40L fusion proteins or negative control, and proliferation, activation, and antibody class switching assessed by flow cytometry. CD40L has been demonstrated to enhance B cell activation, proliferation, and induce antibody class switching.

### Example 11: Stimulation and Activation Differentiation of DCs In Vitro—Primary Cells

Monocytes from normal human PBMCs are differentiated in vitro using standard methodology+/−targeted CD40L or control protein in the presence of CEFT peptide. Flow profiling of DC maturation, activation, and HLA expression are assessed after 7 days and should be enhanced in the CD40L treated conditions.

### Example 12: In Vitro Generation of Antigen-Specific T Cells (DC and T Co-Culture) Tumor Lysate Pulsing and ELISPOT Readout after 2 Weeks

Monocytes from normal human PBMCs are differentiated in vitro using standard methodology+/−targeted CD40L or control protein in the presence of CEFT peptide. After 7 days, autologous T cells are added and cultured for an additional 7 days. At 2 weeks, T cells re-stimulated with CEFT peptide and cytokine responses are assessed via ELISPOT. CD40L fusion proteins should enhance development of antigen-specific memory T cells and result in greater IFNg secretion upon restimulation.

### Example 13: B Cell Activation and Proliferation Tumor TIL (Targeted CD40L)

B cells are isolated from human tumor infiltrating lymphocyte samples by negative selection using a commercially available enrichment kit. Isolated B cells are stimulated with CD40L fusion proteins or negative control, and proliferation, activation, and antibody class switching are assessed by flow cytometry. CD40L has been demonstrated to enhance B cell activation, proliferation, and induce antibody class switching.

### Example 14: In Vivo Efficacy: Dose-Response and Schedule as Monotherapy and in Combination with Checkpoints (aPD-1, aPD-L1, a-CTLA-4, and aLAG3)

### Models in EMT6, B16-OVA, MC38, Etc.

In vivo efficacy is assessed in common syngeneic tumor bearing mice. Naive mice are inoculated with tumor cells s.c. and monitored for tumor growth. When tumors reach ˜150 mm³, mice are randomized to treatment group. Mice are treated with targeted fusion proteins as a monotherapy or in combination with a checkpoint inhibitor. Tumor growth inhibition and survival are assessed for the duration of the study. Fusion proteins as a monotherapy should result in tumor growth inhibition in tested models, and combination with checkpoint inhibitors should result in enhanced efficacy.

### Example 15: In Vivo Efficacy: Combinations Beyond Checkpoints (Cytotoxics)

Similar combinations as experiment 4 but in mice

### Run in Cell-Line Derived Xenografts

In vivo efficacy is assessed in common cell line derived xenograft tumor bearing mice (SCID/nude). Naive mice are inoculated with tumor cells s.c. and monitored for tumor growth. When tumors reach ˜150 mm³, mice are randomized to treatment group. Mice are treated with targeted fusion proteins as a monotherapy or in combination with an ADCC capable mAb (trastuzumab for Her2+ xenografts; cetuximab for EGFR+ xenografts, rituximab for CD20+ xenografts, etc.,). Tumor growth inhibition and survival are assessed for the duration of the study. Fusion proteins as a monotherapy should result in tumor growth inhibition in tested models, and combination with cytotoxic antibodies should result in enhanced efficacy.

### Example 16: In Vivo Efficacy: Combination with Cell Therapies

### NK Cells and CD8 T Cells

### Run in Cell-Line Derived Xenografts

In vivo efficacy is assessed in common cell line derived xenograft tumor bearing mice (SCID/nude). Naive mice are inoculated with tumor cells s.c. and monitored for tumor growth. When tumors reach ˜150 mm³, mice are randomized to treatment group. Mice are infused with CAR-T or CAR-NK cells and treated with targeted fusion proteins or a relevant control protein. Tumor growth inhibition and survival are assessed for the duration of the study. Fusion proteins should enhance anti-tumor activity of CAR-T and CAR-NK cells.

### Example 17: In Vivo Augmentation of Antigen-Specific Immune Responses

OTI Model, Assessment of SIINFEKL Peptide Reactive T Cells when Treated with Targeted Constructs

In vivo activity and development of antigen-specific responses are assessed in OTI mice bearing B16-OVA tumors. Naive mice are inoculated with tumor cells s.c. and monitored for tumor growth. When tumors reach ˜150 mm³, mice are randomized to treatment group. Mice are treated with targeted fusion proteins as a monotherapy. 2-3 weeks following start of dosing, tumors, dLN, and spleen are harvested and assessed for frequency and phenotype of SIINFEKL (OVA peptide sequence) tetramer specific T cells. Fusion proteins should result in heightened induction of OVA-specific T cell responses in treated mice.

### Example 18: PD Assessment Demonstrating In Vivo Activity

### Short-Term (Single Dose or 1 Week Dosing), Assessment of CD4, CD8, B, NK, and DC Activity and Phenotype in Tumor and dLN Following Targeted Ligand Treatment

### Models in EMT6, B16-OVA, MC38, Etc.

In vivo activity and immune cell activation and polarization are assessed in syngeneic tumor-bearing mice. Naive mice are inoculated with tumor cells s.c. and monitored for tumor growth. When tumors reach ˜150 mm³, mice are randomized to treatment group. Mice are treated with targeted fusion proteins as a monotherapy. Following 1 week of dosing, tumors, dLN, and spleen are harvested and assessed for frequency and phenotype of immune cells present in tumors, dLN, and spleens. OX40L, GITRL, and 4-1BBL should stimulate T cell expansion and activation. Additional NK activity should be observed for 4-1BBL, and CD40L should stimulate expansion of macrophages/DCs, B cells, and CD8 T cells.

### Example 19: Assessment of Durability of Response in Cured Mice

Immunocompetent mice cured of syngeneic tumors by treatment with a targeted fusion protein are re-challenged with the same tumor after a 10 week washout period following initial study completion. Tumor growth is measured. Mice previously cured of tumors following fusion protein treatment should spontaneously reject re-inoculated tumors, demonstrating induction of adaptive immune responses and durable anti-tumor immunity during initial treatment.

### Example 20: CTHRC1 Highly Selectively Localizes to Cancer, is Associated with Poor Outcome, and is Most Upregulated on Cancer-Associated Fibroblasts (CAFs) in Immune-Cold Tumor Microenvironments

Without being bound to theory, in the context of cancer, it is expected that inhibiting CTHRC1 may confer therapeutic benefit through blocking CAF and Autocrine pro-survival signalling to cancer cells alongside disrupting immune suppression mediated by CTHRC1. Targeting CTHRC1 with antibodies bound to toxins or that engage immune cells may further drive anti-tumor activity. Data was gathered which supports the idea that CTHRC1 is both selectively upregulated in cancer and contributes to cancer progression.

Binding data (from bio-layer interferometry) for exemplary anti-CTHRC1 antibodies are provided in the table below. anti-CTHRC1 antibodies were screened in the cell adhesion assay to assess for functional activity. Of the 12 clones identified to be selective CTHRC1 binders by ELISA, three clones (CTHRC1S-M5, CTHRC1S-M11 & CTHRC1S-M23) showed functional activity.

| | | BLI | | | | Rat |
|---|---|---|---|---|---|---|
| | | Epitope | Affinity | huCTHRC1 | muCTHRC1 | CTHRC1 |
| Clone | mAb ID | Bin | (nM) | EC50 (nM) | EC50 (nM) | EC50 (nM) |
| CTHRC1S-M5 | AB987 | B | 3.18 | 1.85 | 0.26 | 1.73 |
| CTHRC1S-M23 | AB988 | D | 0.23 | 0.18 | 0.08 | 0.18 |
| CTHRC1S-M11 | AB989 | C | 2.00 | 9.76 | n/a | n/a |
| CTHRC1S-M14 | AB991 | unknown | <0.001 | 0.05 | 0.94 | 0.05 |

CTHRC1 mRNA is a Top Ranked Marker of CAFs in Cancer-Rich, Immune-Cold, Tumour Samples

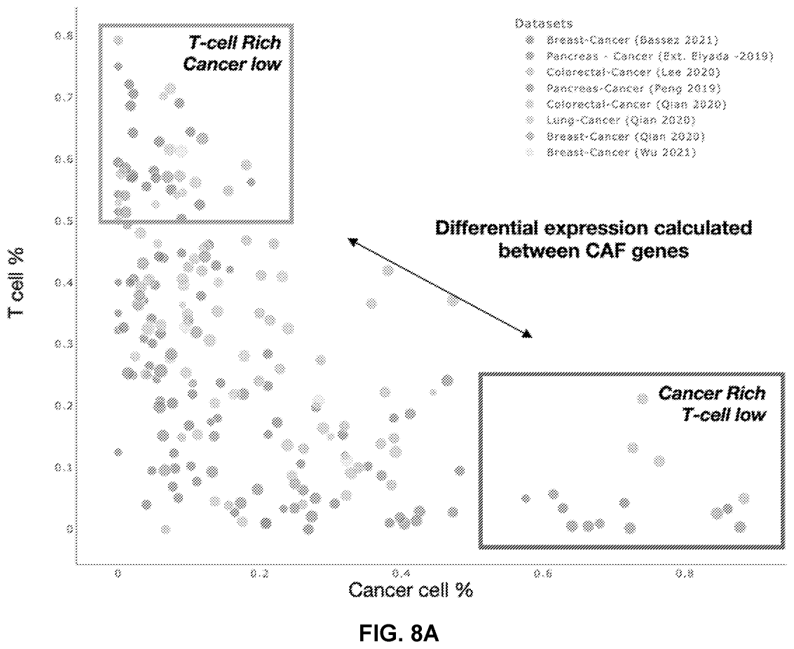

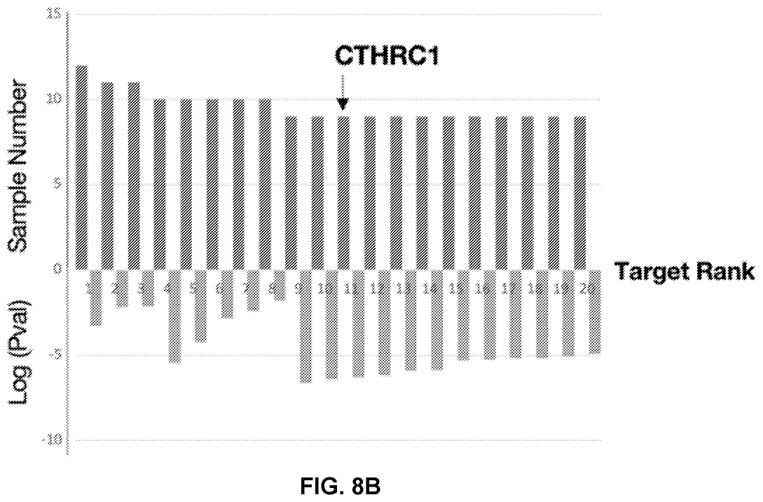

In this example it is demonstrated that CTHRC1 is upregulated specifically in CAFs in cancer rich, immune-cold, samples vs. CAFs in T-cell rich, immune-hot, samples (FIGS. 8A-8B). Cancer rich, T-cell poor tumours, i.e., Immune excluded or Immune deserts, are also associated with poor outcome, treatment resistance, and immune-suppression (Gooden et al. British J. of Cancer, 2011), thus, therapeutic targeting of CTHRC1 represents an opportunity to target these challenging tumor types. Cancer cells transform fibroblasts into CAFs, which in turn promote cancer progression, treatment resistance and immune suppression (Sahai et al. Nat. Rev. Cancer, 2020). Targets that associate closely with CAFs in cancer rich vs. T-cell rich samples are therefore an opportunity for therapeutic intervention in these cancers. To show CTHRC1 is associated with CAFs in these tumor types samples were grouped in the scRNA atlas described in Swechha et al. (bioRxiv 2021) into those that comprise at least 50% cancer cells, and less than 25% T-cells (immune-cold) and those that have over 50% T-cells in the sample and less than 25% cancer cells (immune-hot) (FIG. 8A). Four cancers were included in this analysis, namely, Pancreatic, Lung, Breast, and Colorectal, and are representative of the solid tumours in the scRNA atlas (Swechha et al., bioRxiv, 2021). CAF specific gene expression levels in the Cancer-cell rich, T-cell poor samples were then ranked by the number of samples where expression is significantly greater (P<0.05) than in the T-cell rich, Cancer-cell poor sample; as well as the overall P-value of this finding (Wilcoxon Rank; Navon, Roy, et al., PLoS One, 2009). Finally, targets were filtered down to those upregulated by CAFs but not in other cell types, in all or the majority of samples (Top 500 CAF genes as measured by Wilcoxon Rank). This result demonstrates CTHRC1 is ranked as a target highly localized to CAFs in Cancer-rich, T-cell poor tumour samples (FIG. 8B), and thus an opportunity for therapeutic intervention.

CTHRC1 mRNA is Upregulated in Cancer Vs. Adjacent Tissue and Correlates with Disease Progression

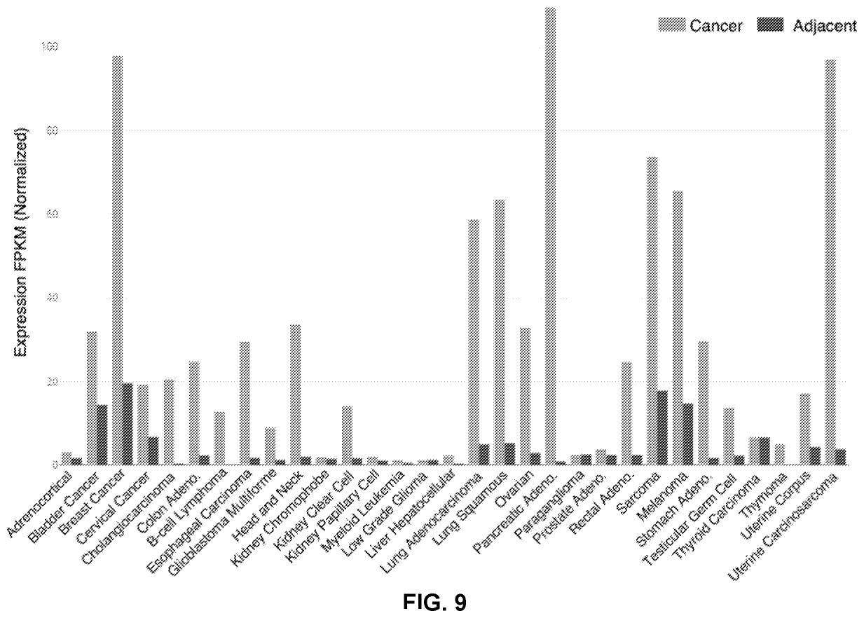

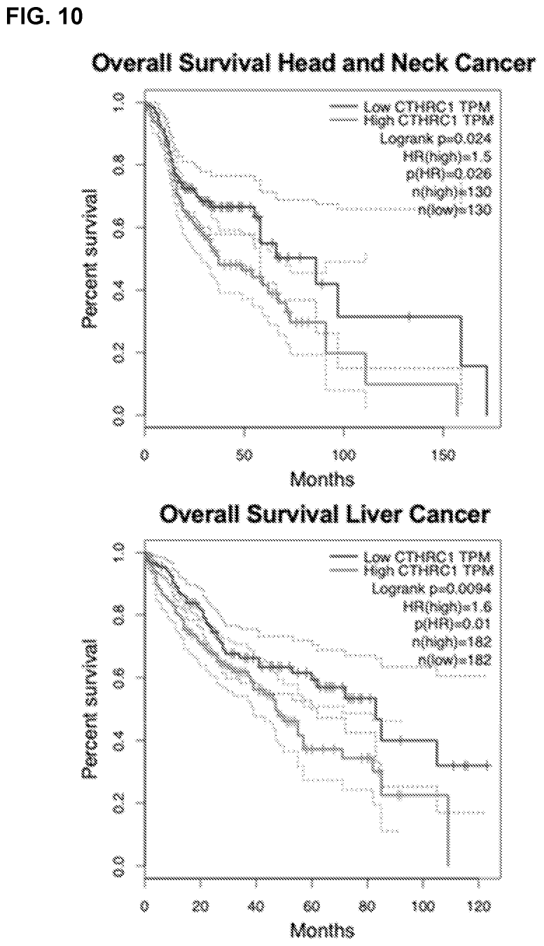

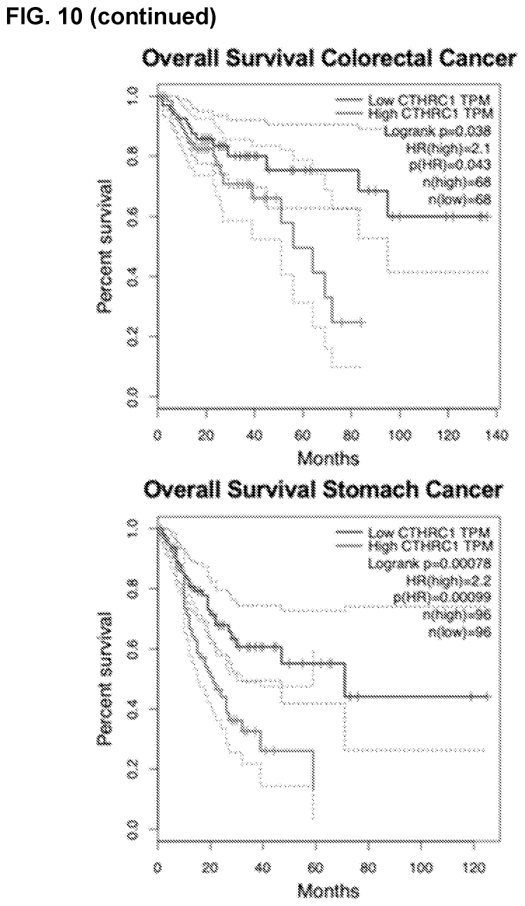

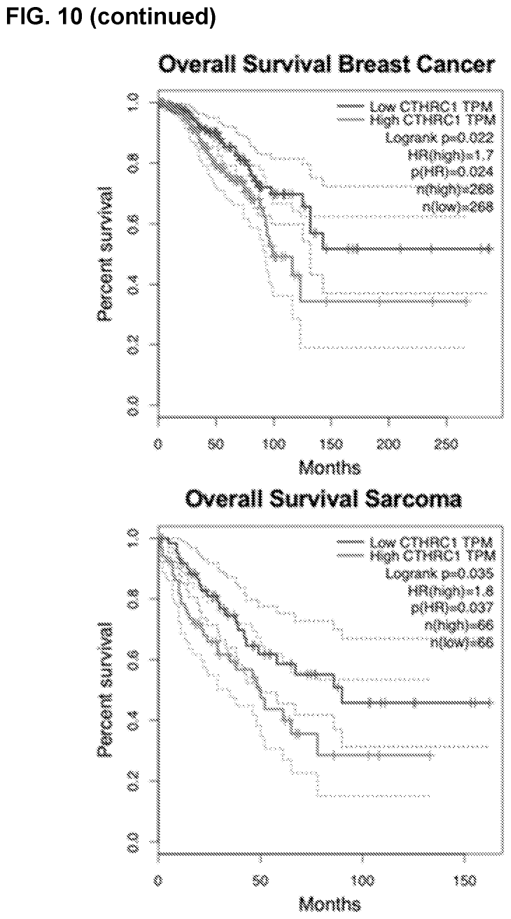

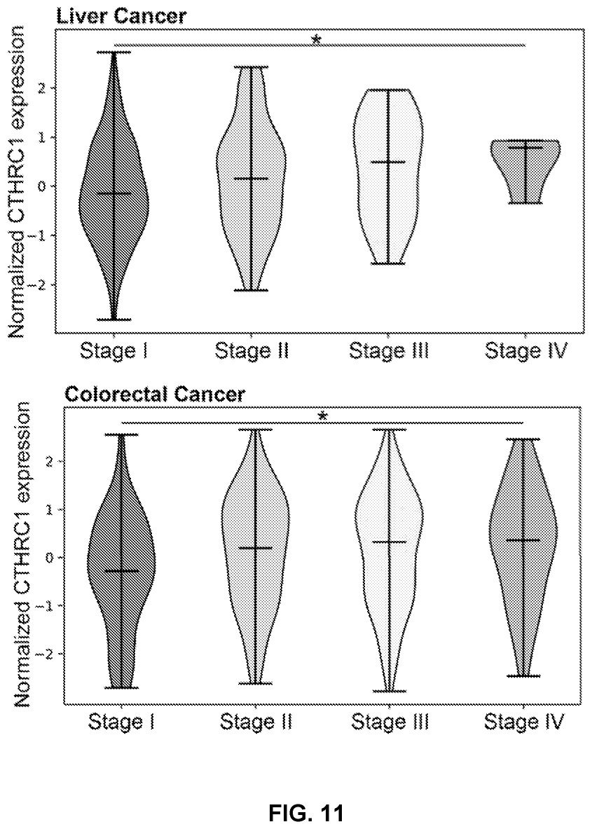

In this Example it is demonstrated that CTHRC1 expression is highly upregulated in many solid tumors. Specifically, an analysis of bulk-scRNA data taken from the cancer genome atlas (TCGA) demonstrates that CTHRC1 is highly upregulated in cancerous tissue samples vs. normal adjacent tissue samples across numerous solid cancers including Breast, Lung, Ovarian, Pancreatic, Sarcoma, Melanoma, and Uterine Carcinosarcomas (FIG. 9). This indicates that within these organs CTHRC1 selectively localizes to cancerous regions. It is also demonstrated that CTHRC1 is a prognostic indicator of survival in many solid cancers based on analysis of TCGA data with the GEPIA online tool (Tang, Z. et al. Nucleic Acids Res, 2017) (FIG. 10). Notably poor survival is seen in patients high for CTHRC1 in liver cancer, stomach cancer, and sarcomas. These cancers are all stromal rich, fibrotic cancers, in line with the above example that shows CTHRC1 localizes to CAFs in cancer-rich immune-poor tumor samples. It is also demonstrated that CTHRC1 expression increases with cancer stage in colorectal cancer and liver cancer (FIG. 11); this shows that targeting CTHRC1 may be valuable in patients with late-stage aggressive cancers and where prognosis is poor.

CTHRC mRNA has a Favorable Expression Profile in Normal Tissue

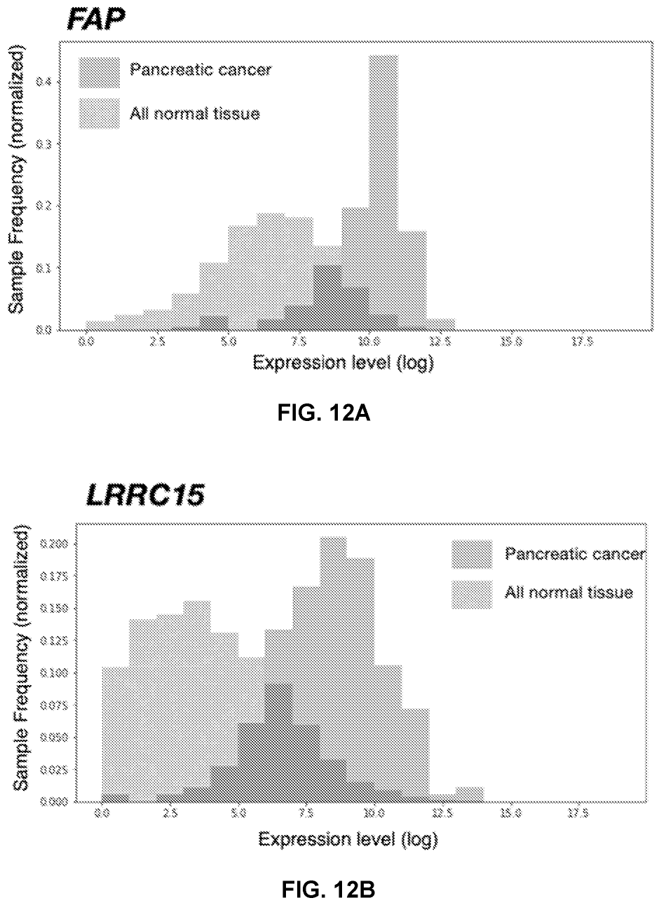

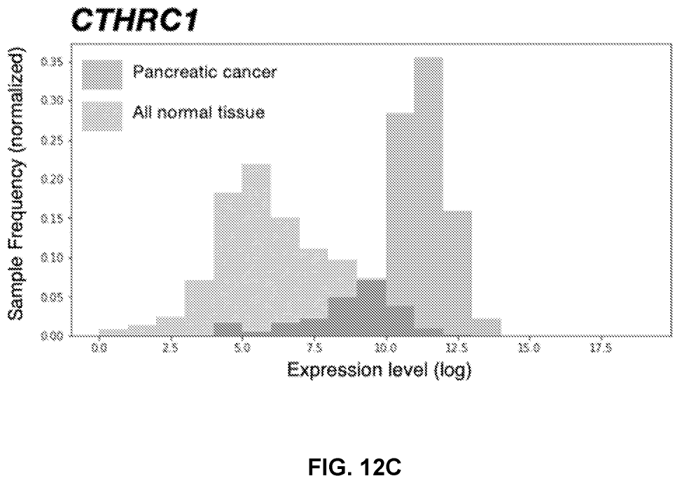

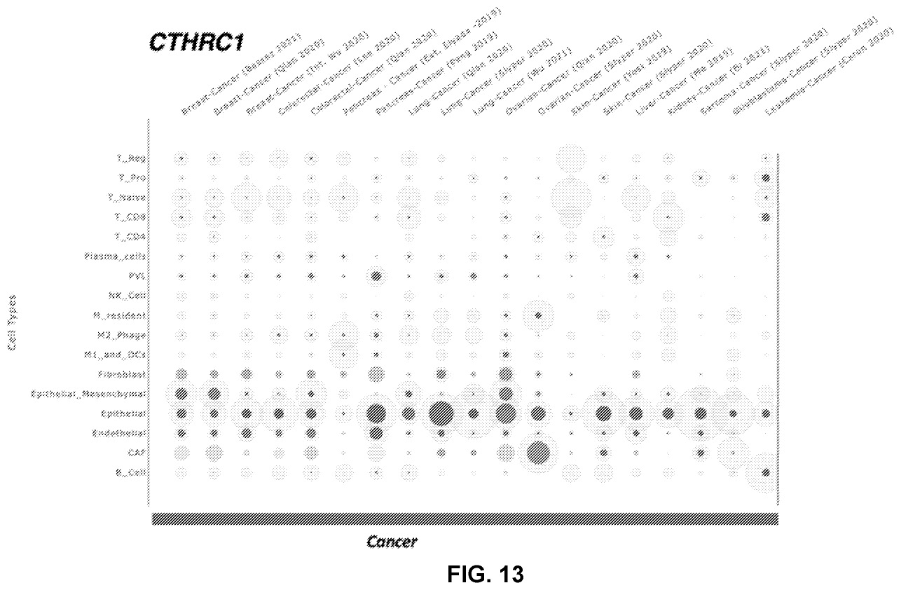

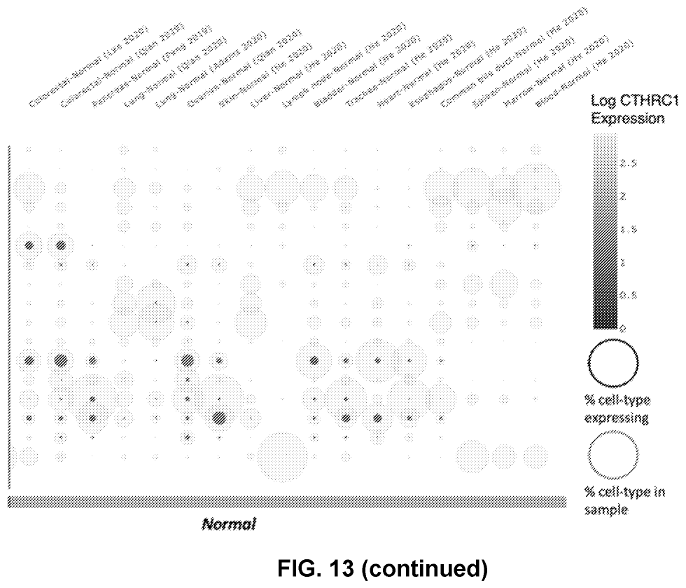

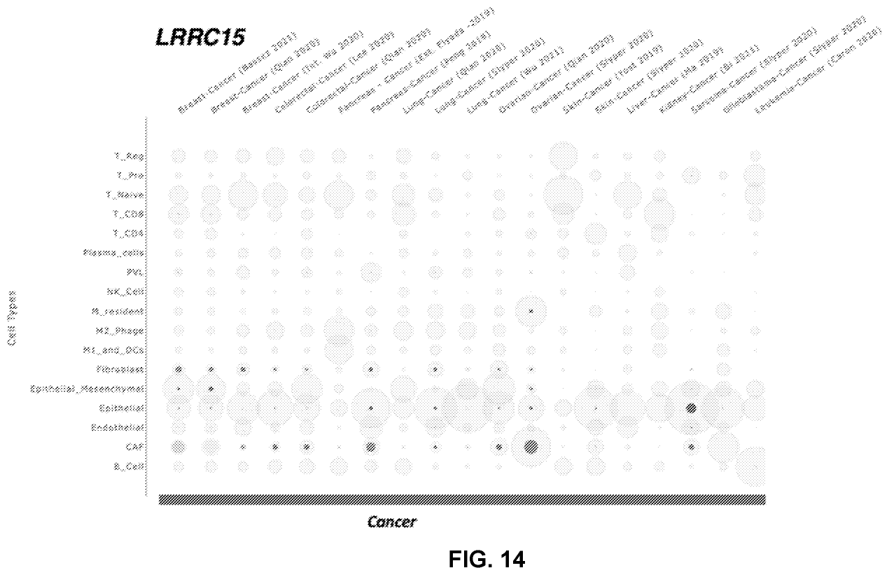

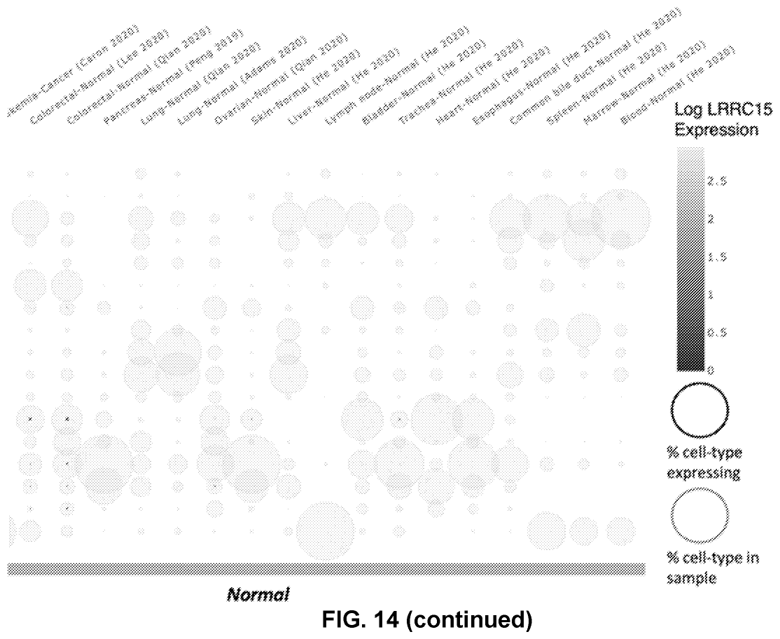

As well as being upregulated in cancer vs. adjacent tissues, this Example also demonstrates that CTHRC1 expression is highly selective to cancer tissue and is expressed at comparatively very low levels in normal healthy tissues in the body. This indicates CTHRC1 targeting is accompanied with a significant therapeutic window and can be used to target payloads to the tumor microenvironment. For example, comparing CTHRC1 bulk-RNA expression in pancreatic cancer samples (TCGA) to CTHRC1 expression in normal tissue samples (GTEX data; both reanalyzed by the UCSC Xena project, Goldman et al., Nat. Biotech, 2020) highlights a significant therapeutic window in almost all pancreatic samples analyzed (FIGS. 12A-12C). At the single-cell level it was seen that CTHRC1 expression localizes to CAFs and epithelial cancer cells in cancer specimens and is not seen in any cell-types in tissue (FIG. 13). This level of localization is comparable if not better than previous mAb targets that have been used to target Antibody Drug Conjugates (ADCs) to the tumour microenvironment and shown safe and non-toxic in clinical trials, such as, LRRC15 (FIG. 14). Of note, in Breast, Ovarian, Pancreatic, and Lung cancers as well as Melanoma we see cancer epithelial cells displaying CTHRC1. Expression is not seen in normal epithelial cells; this indicates a mesenchymal program is switched on in cancer and further implicates CTHRC1 as playing a pro-vs. anti-tumor role, since there is significant evolutionary pressure for cancer cells to down-regulate anti-tumor targets and mechanisms. Overall, this shows CTHRC1 expression is selective enough to cancer vs. normal tissue that mAbs could be used to target payloads to cancer, and that CTHRC1 expression by cancer cells indicates a pro-tumor role for this protein in humans. Collectively, this example therefore shows the value of CTHRC1 mAbs as a method of treating cancer in humans.

### CTHRC1 Protein is Expressed in Cancer-Fibroblast Co-Cultures, and in Mouse Tumours

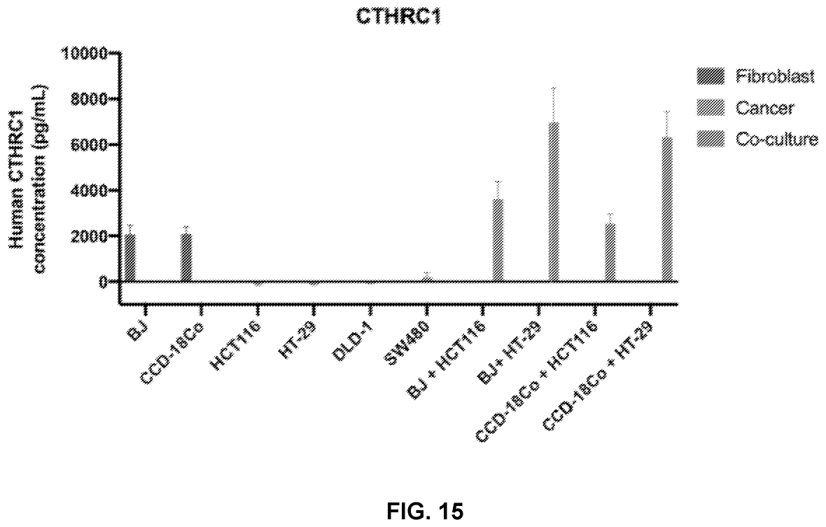

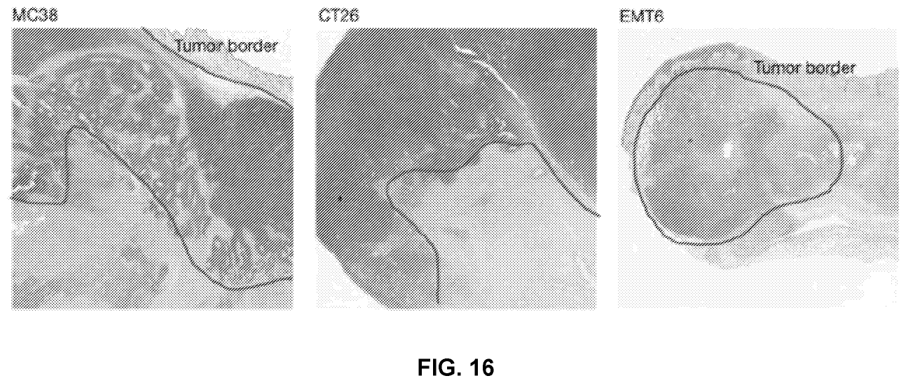

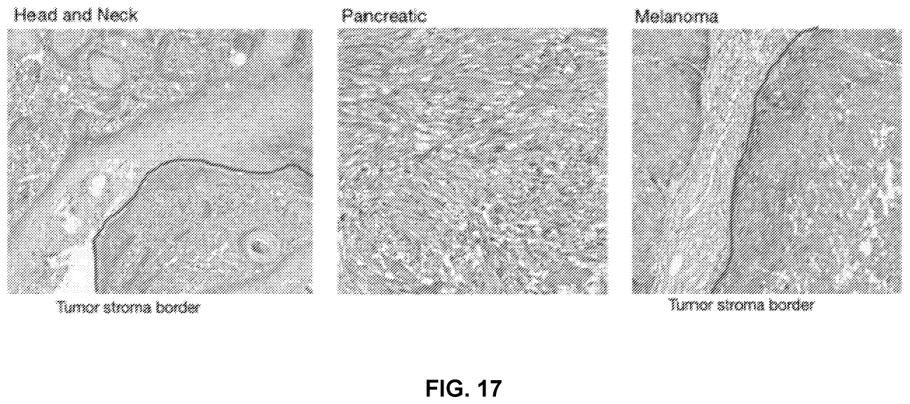

This example also demonstrates that CTHRC1 is upregulated under experimental conditions where fibroblasts are co-cultured with cancer cells versus monocultures of the same cells (FIG. 15), indicating induction of CTHRC1 is dependent on fibroblast-cancer cell interactions, showing specificity to cancer tissue. It was also found in vivo that CTHRC1 protein is selectively expressed in tumor sections using mAbs that selectively bind CTHRC1 (FIG. 16). Finally it was seen that CTHRC1 is expressed on human cancer samples (FIG. 17), either on cancer cells at stromal interfaces (Melanoma and Head and Neck Cancer) or in regions rich in CAFs (Pancreatic Cancer). Overall this analysis shows that at the protein level CTHRC1 expression is seen in cancers similarly to CTHRC1 mRNA.

### Example 21: In Vivo Models

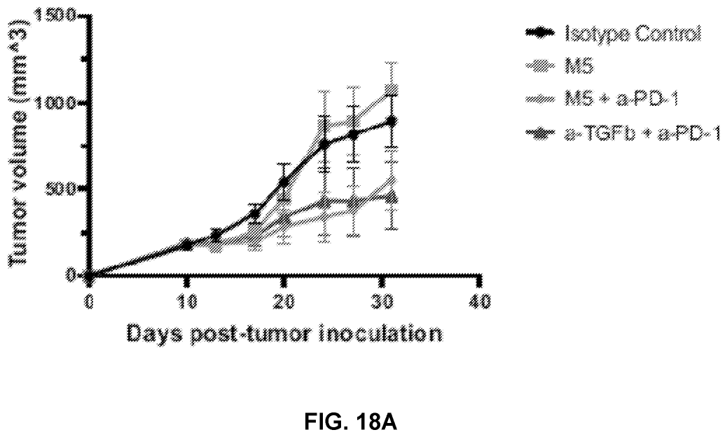

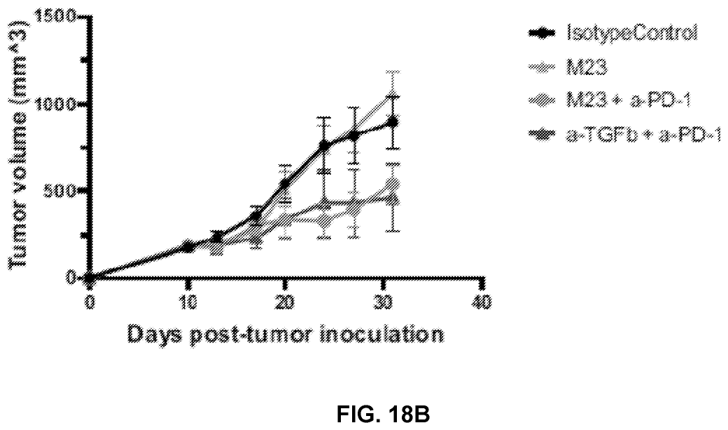

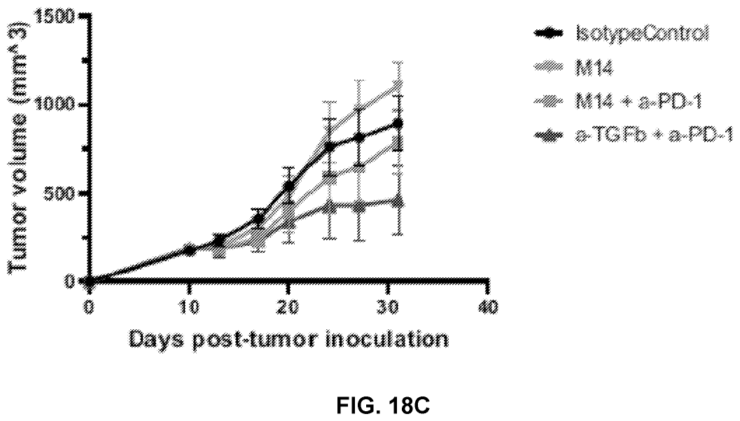

The efficacy of anti-CTHRC1 was tested in syngeneic mouse breast tumor model, EMT6. Briefly, 100,000 EMT6 cells were injected into the mammary fat pad (MFP) of female Balb/c mice. Mice were grouped out according to tumor volume once size reached 120-250 mm³ range, 10 days post inoculation. Following group out, mice were dosed with 2.5 mg/kg anti-CTHRC1 or Isotype control, 5 mg/kg aPD-1, and/or 10 mg/kg a-TGFb (SR) according to group treatment. Tumor volume was assessed twice weekly following caliper measurement and calculated as (length×width²)/2. Initial dose was given intravenous (iv), and remaining doses were administered intraperitoneal (ip) three times a week for three weeks. Mice were euthanized when tumor size exceeds 1500 mm3 or due to tumor ulceration. FIG. 18 illustrates the tumor-growth curves obtained from measurements for 31 days post-tumor cell inoculation. As shown in FIG. 18A, anti-CTHRC1 clone M5 in combination with anti-PD-1 resulted in tumor growth inhibition comparable to that obtained with anti-PD-1 in combination with anti-TGFb. Similar results were obtained for anti-CTHRC1 clone M23 (FIG. 18B) whereas only modest combination activity was observed with anti-CTHRC1 clone M14 in combination with anti-PD-1 (FIG. 18C). Control data are identical for FIGS. 18A-C. Data are plotted as mean+/−standard deviation for each data point across 9 mice per group.

### Example 22: In Vivo Model

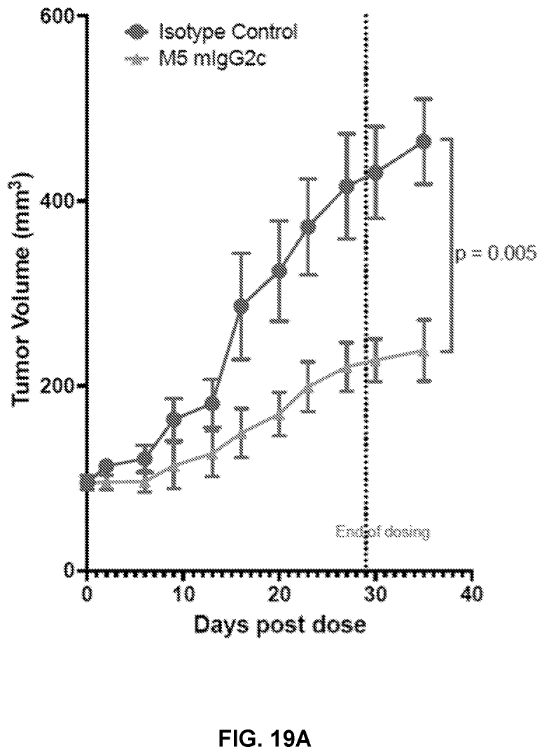

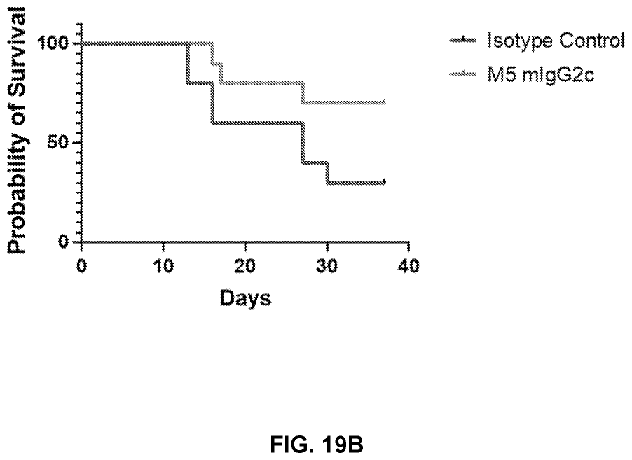

Female C57BL/6J mice were inoculated s.c. with Pan02 cells in Matrigel. Tumors were measured, and mice were randomized to treatment group when tumors reached an average volume of 100 mm³. Treatment with 10 mg/kg of isotype control or anti-CTHRC1 mAb (clone M5) began 24 hours post-randomization and continued for 3 doses/week for the indicated treatment duration. Mice were monitored for tumor growth (FIG. 19A) and overall survival (FIG. 19B). Tumor grown inhibition was deemed significantly significant by ANOVA. As shown in FIG. 19A, anti-CTHRC1 clone M5 resulted in tumor growth inhibition relative to isotype control. Data are plotted as mean tumor volume+/−standard deviation for each data point across 9 mice per group. As shown in FIG. 19B, anti-CTHRC1 clone M5 resulted in improved survival relative to isotype control.

### Example 23: Anti-CTHRC1 Pretreatment Results in Recruitment of CD8 T Cells in the Tumor Microenvironment

Balb/c mice were inoculated orthotopically with EMT6 tumor cells in Matrigel. When tumors reached an average volume of 200 mm³, animals were assigned to treatment group. Mice were treated with isotype or anti-CTHRC1 mAb (M5 clone) at 10 mg/kg for 1 week (3 doses).

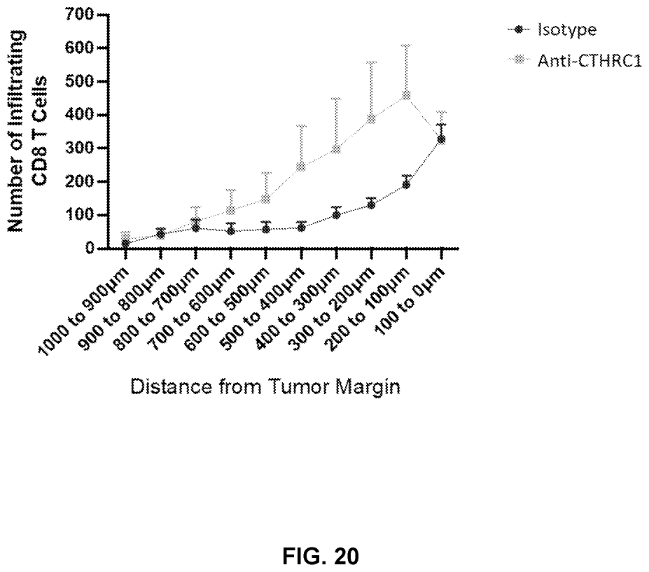

Following dosing, tumors were isolated and processed to slides. Slides were stained with an anti-CD8 antibody, and level of CD8 infiltration into tumor nests was quantified by HALO image analysis software. Data were plotted as number of infiltrating CD8 T cells versus distance from tumor margin (FIG. 20). As shown in FIG. 21A, pretreatment of cells with an anti-CTHRC1 antibody resulted in improved CD8 T cell recruitment in the tumor microenvironment. These results suggest that preconditioning treatment with an anti-CTHRC1 antibody of the invention can result in CD8 T cell recruitment that could be advantageous for cellular immunotherapies such as CAR-T cell therapy.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

SEQUENCE LISTING

| SEQ ID NO | SEQUENCE |
|---|---|
| 1 | MEYASDASLDPEAPWPPAPRARACRVLPWALVAGLLLLLLLAAAC |
| (human 4- | AVFLACPWAVSGARASPGSAASPRLREGPELSPDDPAGLLDLRQGM |
| 1BBL, full | FAQLVAQNVLLIDGPLSWYSDPGLAGVSLTGGLSYKEDTKELVVAK |
| length, Uniprot | AGVYYVFFQLELRRVVAGEGSGSVSLALHLQPLRSAAGAAALALT |
| P41273) | VDLPPASSEARNSAFGFQGRLLHLSAGQRLGVHLHTEARARHAWQ |
| | LTQGATVLGLFRVTPEIPAGLPSPRSE |
| 2 | ACPWAVSGARASPGSAASPRLREGPELSPDDPAGLLDLRQGMFAQL |
| (human 4- | VAQNVLLIDGPLSWYSDPGLAGVSLTGGLSYKEDTKELVVAKAGV |
| 1BBL, | YYVFFQLELRRVVAGEGSGSVSLALHLQPLRSAAGAAALALTVDLP |
| ectodomain, aa | PASSEARNSAFGFQGRLLHLSAGQRLGVHLHTEARARHAWQLTQG |
| 50-254) | ATVLGLFRVTPEIPAGLPSPRSE |
| 3 | PWAVSGARASPGSAASPRLREGPELSPDDPAGLLDLRQGMFAQLVA |
| (human 4- | QNVLLIDGPLSWYSDPGLAGVSLTGGLSYKEDTKELVVAKAGVYY |
| 1BBL, aa 52- | VFFQLELRRVVAGEGSGSVSLALHLQPLRSAAGAAALALTVDLPPA |
| 254) | SSEARNSAFGFQGRLLHLSAGQRLGVHLHTEARARHAWQLTQGAT |
| | VLGLFRVTPEIPAGLPSPRSE |
| 4 | PWAVSGARASPGSAASPRLREGPELSPDDPAGLLDLRQGMFAQLVA |
| (human 4- | QNVLLIDGPLSWYSDPGLAGVSLTGGLSYKEDTKELVVAKAGVYY |
| 1BBL, aa 52- | VFFQLELRRVVAGEGSGSVSLALHLQPLRSAAGAAALALTVDLPPA |
| 248) | SSEARNSAFGFQGRLLHLSAGQRLGVHLHTEARARHAWQLTQGAT |
| | VLGLFRVTPEIPAGL |
| 5 | REGPELSPDDPAGLLDLRQGMFAQLVAQNVLLIDGPLSWYSDPGLA |
| (human 4- | GVSLTGGLSYKEDTKELVVAKAGVYYVFFQLELRRVVAGEGSGSV |
| 1BBL, aa 71- | SLALHLQPLRSAAGAAALALTVDLPPASSEARNSAFGFQGRLLHLS |
| 254) | AGQRLGVHLHTEARARHAWQLTQGATVLGLFRVTPEIPAGLPSPRS |
| | E |
| 6 | REGPELSPDDPAGLLDLRQGMFAQLVAQNVLLIDGPLSWYSDPGLA |
| (human, 4- | GVSLTGGLSYKEDTKELVVAKAGVYYVFFQLELRRVVAGEGSGSV |
| 1BBL, aa 71- | SLALHLQPLRSAAGAAALALTVDLPPASSEARNSAFGFQGRLLHLS |
| 248) | AGQRLGVHLHTEARARHAWQLTQGATVLGLFRVTPEIPAGL |
| 7 | DPAGLLDLRQGMFAQLVAQNVLLIDGPLSWYSDPGLAGVSLTGGL |
| (human, 4- | SYKEDTKELVVAKAGVYYVFFQLELRRVVAGEGSGSVSLALHLQP |
| 1BBL, aa 80- | LRSAAGAAALALTVDLPPASSEARNSAFGFQGRLLHLSAGQRLGVH |
| 254) | LHTEARARHAWQLTQGATVLGLFRVTPEIPAGLPSPRSE |
| 8 | DPAGLLDLRQGMFAQLVAQNVLLIDGPLSWYSDPGLAGVSLTGGL |
| (human, 4- | SYKEDTKELVVAKAGVYYVFFQLELRRVVAGEGSGSVSLALHLQP |
| 1BBL, aa 80- | LRSAAGAAALALTVDLPPASSEARNSAFGFQGRLLHLSAGQRLGVH |
| 248) | LHTEARARHAWQLTQGATVLGLFRVTPEIPAGL |
| 9 | LDLRQGMFAQLVAQNVLLIDGPLSWYSDPGLAGVSLTGGLSYKED |
| (human, 4- | TKELVVAKAGVYYVFFQLELRRVVAGEGSGSVSLALHLQPLRSAA |
| 1BBL, aa 85- | GAAALALTVDLPPASSEARNSAFGFQGRLLHLSAGQRLGVHLHTEA |
| 254) | RARHAWQLTQGATVLGLFRVTPEIPAGLPSPRSE |
| 10 | LDLRQGMFAQLVAQNVLLIDGPLSWYSDPGLAGVSLTGGLSYKED |
| (human, 4- | TKELVVAKAGVYYVFFQLELRRVVAGEGSGSVSLALHLQPLRSAA |
| 1BBL, aa 85- | GAAALALTVDLPPASSEARNSAFGFQGRLLHLSAGQRLGVHLHTEA |
| 248) | RARHAWQLTQGATVLGLFRVTPEIPAGL |
| 11 | MERVQPLEENVGNAARPRFERNKLLLVASVIQGLGLLLCFTYICLHF |
| (human OX40L | SALQVSHRYPRIQSIKVQFTEYKKEKGFILTSQKEDEIMKVQNNSVII |
| (CD252), full | NCDGFYLISLKGYFSQEVNISLHYQKDEEPLFQLKKVRSVNSLMVAS |
| length) | LTYKDKVYLNVTTDNTSLDDFHVNGGELILIHQNPGEFCVL |
| 12 | QVSHRYPRIQSIKVQFTEYKKEKGFILTSQKEDEIMKVQNNSVIINCD |
| (human OX40L | GFYLISLKGYFSQEVNISLHYQKDEEPLFQLKKVRSVNSLMVASLTY |
| (CD252), | KDKVYLNVTTDNTSLDDFHVNGGELILIHQNPGEFCVL |
| ectodomain, aa | |
| 51-183) | |
| 13 | VSHRYPRIQSIKVQFTEYKKEKGFILTSQKEDEIMKVQNNSVIINCDG |
| (human OX40L | FYLISLKGYFSQEVNISLHYQKDEEPLFQLKKVRSVNSLMVASLTYK |
| (CD252), aa 52- | DKVYLNVTTDNTSLDDFHVNGGELILIHQNPGEFCVL |
| 183) | |
| 14 | MIETYNQTSPRSAATGLPISMKIFMYLLTVFLITQMIGSALFAVYLHR |
| (human CD40L, | RLDKIEDERNLHEDFVFMKTIQRCNTGERSLSLLNCEEIKSQFEGFV |
| full length) | KDIMLNKEETKKENSFEMQKGDQNPQIAAHVISEASSKTTSVLQWA |
| | EKGYYTMSNNLVTLENGKQLTVKRQGLYYIYAQVTFCSNREASSQ |
| | APFIASLCLKSPGRFERILLRAANTHSSAKPCGQQSIHLGGVFELQPG |
| | ASVFVNVTDPSQVSHGTGFTSFGLLKL |
| 15 | HRRLDKIEDERNLHEDFVFMKTIQRCNTGERSLSLLNCEEIKSQFEGF |
| (human, | VKDIMLNKEETKKENSFEMQKGDQNPQIAAHVISEASSKTTSVLQW |
| CD40L, | AEKGYYTMSNNLVTLENGKQLTVKRQGLYYIYAQVTFCSNREASS |
| ectodomain, aa | QAPFIASLCLKSPGRFERILLRAANTHSSAKPCGQQSIHLGGVFELQP |
| 47-261) | GASVFVNVTDPSQVSHGTGFTSFGLLKL |
| 16 | LQWAEKGYYTMSNNLVTLENGKQLTVKRQGLYYIYAQVTFCSNRE |
| (human CD40L, | ASSQAPFIASLCLKSPGRFERILLRAANTHSSAKPCGQQSIHLGGVFE |
| aa 138-261) | LQPGASVFVNVTDPSQVSHGTGFTSFGLLKL |
| 17 | MTLHPSPITCEFLFSTALISPKMCLSHLENMPLSHSRTQGAQRSSWK |
| (human GITRL, | LWLFCSIVMLLFLCSFSWLIFIFLQLETAKEPCMAKFGPLPSKWQMA |
| full length) | SSEPPCVNKVSDWKLEILQNGLYLIYGQVAPNANYNDVAPFEVRLY |
| | KNKDMIQTLINKSKIQNVGGTYELHVGDTIDLIFNSEHQVLKNNTY |
| | WGIILLANPQFIS |
| 18 | QLETAKEPCMAKFGPLPSKWQMASSEPPCVNKVSDWKLEILQNGL |
| (human GITRL, | YLIYGQVAPNANYNDVAPFEVRLYKNKDMIQTLINKSKIQNVGGT |
| ectodomain, 72- | YELHVGDTIDLIFNSEHQVLKNNTYWGIILLANPQFIS |
| 199) | |
| 19 | MPEEGSGCSVRRRPYGCVLRAALVPLVAGLVICLVVCIQRFAQAQQ |
| (human CD70, | QLPLESLGWDVAELQLNHTGPQQDPRLYWQGGPALGRSFLHGPEL |
| full length) | DKGQLRIHRDGIYMVHIQVTLAICSSTTASRHHPTTLAVGICSPASRS |
| | ISLLRLSFHQGCTIASQRLTPLARGDTLCTNLTGTLLPSRNTDETFFG |
| | VQWVRP |
| 20 | QRFAQAQQQLPLESLGWDVAELQLNHTGPQQDPRLYWQGGPALG |
| (human CD70, | RSFLHGPELDKGQLRIHRDGIYMVHIQVTLAICSSTTASRHHPTTLA |
| ectodomain, aa | VGICSPASRSISLLRLSFHQGCTIASQRLTPLARGDTLCTNLTGTLLPS |
| 39-193) | RNTDETFFGVQWVRP |
| 21 | MEESVVRPSVFVVDGQTDIPFTRLGRSHRRQSCSVARVGLGLLLLL |
| (human LIGHT, | MGAGLAVQGWFLLQLHWRLGEMVTRLPDGPAGSWEQLIQERRSH |
| full length) | EVNPAAHLTGANSSLTGSGGPLLWETQLGLAFLRGLSYHDGALVVT |
| | KAGYYYIYSKVQLGGVGCPLGLASTITHGLYKRTPRYPEELELLVSQ |
| | QSPCGRATSSSRVWWDSSFLGGVVHLEAGEKVVVRVLDERLVRLR |
| | DGTRSYFGAFMV |
| 22 | LQLHWRLGEMVTRLPDGPAGSWEQLIQERRSHEVNPAAHLTGANS |
| (human LIGHT, | SLTGSGGPLLWETQLGLAFLRGLSYHDGALVVTKAGYYYIYSKVQL |
| ectodomain, aa | GGVGCPLGLASTITHGLYKRTPRYPEELELLVSQQSPCGRATSSSRV |
| 59-240) | WWDSSFLGGVVHLEAGEKVVVRVLDERLVRLRDGTRSYFGAFMV |
| 23 | DGPAGSWEQLIQERRSHEVNPAAHLTGANSSLTGSGGPLLWETQLG |
| (human LIGHT, | LAFLRGLSYHDGALVVTKAGYYYIYSKVQLGGVGCPLGLASTITHG |
| ectodomain, aa | LYKRTPRYPEELELLVSQQSPCGRATSSSRVWWDSSFLGGVVHLEA |
| 74-240) | GEKVVVRVLDERLVRLRDGTRSYFGAFMV |
| 24 | MDPGLQQALNGMAPPGDTAMHVPAGSVASHLGTTSRSYFYLTTAT |
| (human CD30L, | LALCLVFTVATIMVLVVQRTDSIPNSPDNVPLKGGNCSEDLLCILKR |
| full length) | APFKKSWAYLQVAKHLNKTKLSWNKDGILHGVRYQDGNLVIQFPG |
| | LYFIICQLQFLVQCPNNSVDLKLELLINKHIKKQALVTVCESGMQTK |
| | HVYQNLSQFLLDYLQVNTTISVNVDTFQYIDTSTFPLENVLSIFLYSN |
| | SD |
| 25 | QRTDSIPNSPDNVPLKGGNCSEDLLCILKRAPFKKSWAYLQVAKHL |
| (human CD30L, | NKTKLSWNKDGILHGVRYQDGNLVIQFPGLYFIICQLQFLVQCPNNS |
| ectodomain, aa | VDLKLELLINKHIKKQALVTVCESGMQTKHVYQNLSQFLLDYLQV |
| 63-234) | NTTISVNVDTFQYIDTSTFPLENVLSIFLYSNSD |
| 26 | MAMMEVQGGPSLGQTCVLIVIFTVLLQSLCVAVTYVYFTNELKQM |
| (human TRAIL, | QDKYSKSGIACFLKEDDSYWDPNDEESMNSPCWQVKWQLRQLVR |
| from Uniprot | KMILRTSEETISTVQEKQQNISPLVRERGPQRVAAHITGTRGRSNTLS |
| P50591, full | SPNSKNEKALGRKINSWESSRSGHSFLSNLHLRNGELVIHEKGFYYI |
| length) | YSQTYFRFQEEIKENTKNDKQMVQYIYKYTSYPDPILLMKSARNSC |
| | WSKDAEYGLYSIYQGGIFELKENDRIFVSVTNEHLIDMDHEASFFGA |
| | FLVG |
| 27 | TNELKQMQDKYSKSGIACFLKEDDSYWDPNDEESMNSPCWQVKW |
| (human TRAIL, | QLRQLVRKMILRTSEETISTVQEKQQNISPLVRERGPQRVAAHITGT |
| ectodomain, aa | RGRSNTLSSPNSKNEKALGRKINSWESSRSGHSFLSNLHLRNGELVI |
| 39-281) | HEKGFYYIYSQTYFRFQEEIKENTKNDKQMVQYIYKYTSYPDPILLM |
| | KSARNSCWSKDAEYGLYSIYQGGIFELKENDRIFVSVTNEHLIDMDH |
| | EASFFGAFLVG |
| 28 | MAEDLGLSFGETASVEMLPEHGSCRPKARSSSARWALTCCLVLLPF |
| (human TL1A, | LAGLTTYLLVSQLRAQGEACVQFQALKGQEFAPSHQQVYAPLRAD |
| full length) | GDKPRAHLTVVRQTPTQHFKNQFPALHWEHELGLAFTKNRMNYTN |
| | KELLIPESGDYFIYSQVTFRGMTSECSEIRQAGRPNKPDSITVVITKVT |
| | DSYPEPTQLLMGTKSVCEVGSNWFQPIYLGAMFSLQEGDKLMVNV |
| | SDISLVDYTKEDKTFFGAFLL |
| 29 | DKPRAHLTVVRQTPTQHFKNQFPALHWEHELGLAFTKNRMNYTNK |
| (human TL1A, | FLLIPESGDYFIYSQVTFRGMTSECSEIRQAGRPNKPDSITVVITKVTD |
| ectodomain, aa | SYPEPTQLLMGTKSVCEVGSNWFQPIYLGAMFSLQEGDKLMVNVS |
| 93-251) | DISLVDYTKEDKTFFGAFLL |
| 30 | MRLGSPGLLFLLFSSLRADTQEKEVRAMVGSDVELSCACPEGSRFD |
| (human ICOSL, | LNDVYVYWQTSESKTVVTYHIPQNSSLENVDSRYRNRALMSPAGM |
| from Uniprot | LRGDFSLRLFNVTPQDEQKFHCLVLSQSLGFQEVLSVEVTLHVAAN |
| O75144, full | FSVPVVSAPHSPSQDELTFTCTSINGYPRPNVYWINKTDNSLLDQAL |
| length) | QNDTVFLNMRGLYDVVSVLRIARTPSVNIGCCIENVLLQQNLTVGS |
| | QTGNDIGERDKITENPVSTGEKNAATWSILAVLCLLVVVAVAIGWV |
| | CRDRCLQHSYAGAWAVSPETELTGHV |
| 31 | DTQEKEVRAMVGSDVELSCACPEGSRFDLNDVYVYWQTSESKTVV |
| (human ICOSL, | TYHIPQNSSLENVDSRYRNRALMSPAGMLRGDFSLRLFNVTPQDEQ |
| ectodomain, aa | KFHCLVLSQSLGFQEVLSVEVTLHVAANFSVPVVSAPHSPSQDELTF |
| 19-256) | TCTSINGYPRPNVYWINKTDNSLLDQALQNDTVFLNMRGLYDVVS |
| | VLRIARTPSVNIGCCIENVLLQQNLTVGSQTGNDIGERDKITENPVST |
| | GEKNAAT |
| 32 | MGHTRRQGTSPSKCPYLNFFQLLVLAGLSHFCSGVIHVTKEVKEVA |
| (human CD80, | TLSCGHNVSVEELAQTRIYWQKEKKMVLTMMSGDMNIWPEYKNR |
| from Uniprot | TIFDITNNLSIVILALRPSDEGTYECVVLKYEKDAFKREHLAEVTLSV |
| P33681, full | KADFPTPSISDFEIPTSNIRRIICSTSGGFPEPHLSWLENGEELNAINTT |
| length) | VSQDPETELYAVSSKLDFNMTTNHSFMCLIKYGHLRVNQTFNWNT |
| | TKQEHFPDNLLPSWAITLISVNGIFVICCLTYCFAPRCRERRRNERLR |
| | RESVRPV |
| 33 | VIHVTKEVKEVATLSCGHNVSVEELAQTRIYWQKEKKMVLTMMSG |
| (human CD80, | DMNIWPEYKNRTIFDITNNLSIVILALRPSDEGTYECVVLKYEKDAF |
| ectodomain, aa | KREHLAEVTLSVKADFPTPSISDFEIPTSNIRRIICSTSGGFPEPHLSWL |
| 35-242) | ENGEELNAINTTVSQDPETELYAVSSKLDFNMTTNHSFMCLIKYGH |
| | LRVNQTFNWNTTKQEHFPDN |
| 34 | MDPQCTMGLSNILFVMAFLLSGAAPLKIQAYFNETADLPCQFANSQ |
| (human CD86, | NQSLSELVVFWQDQENLVLNEVYLGKEKFDSVHSKYMGRTSFDSD |
| from Uniprot | SWTLRLHNLQIKDKGLYQCIIHHKKPTGMIRIHQMNSELSVLANFSQ |
| P42081, full | PEIVPISNITENVYINLTCSSIHGYPEPKKMSVLLRTKNSTIEYDGVMQ |
| length) | KSQDNVTELYDVSISLSVSFPDVTSNMTIFCILETDKTRLLSSPFSIEL |
| | EDPQPPPDHIPWITAVLPTVIICVMVFCLILWKWKKKKRPRNSYKCG |
| | TNTMEREESEQTKKREKIHIPERSDEAQRVFKSSKTSSCDKSDTCF |
| 35 | APLKIQAYFNETADLPCQFANSQNQSLSELVVFWQDQENLVLNEVY |
| (human CD86, | LGKEKFDSVHSKYMGRTSFDSDSWTLRLHNLQIKDKGLYQCIIHHK |
| ectodomain, aa | KPTGMIRIHQMNSELSVLANFSQPEIVPISNITENVYINLTCSSIHGYP |
| 24-247) | EPKKMSVLLRTKNSTIEYDGVMQKSQDNVTELYDVSISLSVSFPDVT |
| | SNMTIFCILETDKTRLLSSPFSIELEDPQPPPDHIP |
| 36 | MSKEPLILWLMIEFWWLYLTPVTSETVVTEVLGHRVTLPCLYSSWS |
| (human TIM-4, | HNSNSMCWGKDQCPYSGCKEALIRTDGMRVTSRKSAKYRLQGTIP |
| from Uniprot | RGDVSLTILNPSESDSGVYCCRIEVPGWFNDVKINVRLNLQRASTTT |
| Q96H15-1, full | HRTATTTTRRTTTTSPTTTRQMTTTPAALPTTVVTTPDLTTGTPLQM |
| length) | TTIAVFTTANTCLSLTPSTLPEEATGLLTPEPSKEGPILTAESETVLPS |
| | DSWSSVESTSADTVLLTSKESKVWDLPSTSHVSMWKTSDSVSSPQP |
| | GASDTAVPEQNKTTKTGQMDGIPMSMKNEMPISQLLMIIAPSLGFV |
| | LFALFVAFLLRGKLMETYCSQKHTRLDYIGDSKNVLNDVQHGRED |
| | EDGLFTL |
| 37 | MDPKGLLSLTFVLFLSLAFGASYGTGGRMMNCPKILRQLGSKVLLP |
| (human | LTYERINKSMNKSIHIVVTMAKSLENSVENKIVSLDPSEAGPPRYLG |
| SLAMF1, from | DRYKFYLENLTLGIRESRKEDEGWYLMTLEKNVSVQRFCLQLRLYE |
| Uniprot | QVSTPEIKVLNKTQENGTCTLILGCTVEKGDHVAYSWSEKAGTHPL |
| Q13291, full | NPANSSHLLSLTLGPQHADNIYICTVSNPISNNSQTFSPWPGCRTDPS |
| length) | ETKPWAVYAGLLGGVIMILIMVVILQLRRRGKTNHYQTTVEKKSLT |
| | IYAQVQKPGPLQKKLDSFPAQDPCTTIYVAATEPVPESVQETNSITV |
| | YASVTLPES |
| 38 | ASYGTGGRMMNCPKILRQLGSKVLLPLTYERINKSMNKSIHIVVTM |
| (human | AKSLENSVENKIVSLDPSEAGPPRYLGDRYKFYLENLTLGIRESRKE |
| SLAMF1, | DEGWYLMTLEKNVSVQRFCLQLRLYEQVSTPEIKVLNKTQENGTCT |
| ectodomain, aa | LILGCTVEKGDHVAYSWSEKAGTHPLNPANSSHLLSLTLGPQHADN |
| 21-237) | IYICTVSNPISNNSQTFSPWPGCRTDPSETKP |
| 39 | MVAGSDAGRALGVLSVVCLLHCFGFISCFSQQIYGVVYGNVTFHVP |
| (human CD58, | SNVPLKEVLWKKQKDKVAELENSEFRAFSSFKNRVYLDTVSGSLTI |
| from Uniprot | YNLTSSDEDEYEMESPNITDTMKFFLYVLESLPSPTLTCALTNGSIEV |
| P19256, full | QCMIPEHYNSHRGLIMYSWDCPMEQCKRNSTSIYFKMENDLPQKIQ |
| length) | CTLSNPLFNTTSSIILTTCIPSSGHSRHRYALIPIPLAVITTCIVLYMNGI |
| | LKCDRKPDRTNSN |
| 40 | FSQQIYGVVYGNVTFHVPSNVPLKEVLWKKQKDKVAELENSEFRA |
| (human CD58, | FSSFKNRVYLDTVSGSLTIYNLTSSDEDEYEMESPNITDTMKFFLYV |
| ectodomain, aa | |
| 29-120) | |
| 41 | MLWLFQSLLFVFCFGPGNVVSQSSLTPLMVNGILGESVTLPLEFPAG |
| (human | EKVNFITWLFNETSLAFIVPHETKSPEIHVTNPKQGKRLNFTQSYSLQ |
| SLAMF6, from | LSNLKMEDTGSYRAQISTKTSAKLSSYTLRILRQLRNIQVINHSQLF |
| Uniprot | QNMTCELHLTCSVEDADDNVSFRWEALGNTLSSQPNLTVSWDPRIS |
| Q96DU3, full | SEQDYTCIAENAVSNLSFSVSAQKLCEDVKIQYTDTKMILFMVSGICI |
| length) | VFGFIILLLLVLRKRRDSLSLSTQRTQGPAESARNLEYVSVSPTNNTV |
| | YASVTHSNRETEIWTPRENDTITIYSTINHSKESKPTFSRATALDNVV |
| 42 | QSSLTPLMVNGILGESVTLPLEFPAGEKVNFITWLFNETSLAFIVPHE |
| (human | TKSPEIHVTNPKQGKRLNFTQSYSLQLSNLKMEDTGSYRAQISTKTS |
| SLAMF6, | AKLSSYTLRILRQLRNIQVTNHSQLFQNMTCELHLTCSVEDADDNV |
| ectodomain, aa | SFRWEALGNTLSSQPNLTVSWDPRISSEQDYTCIAENAVSNLSFSVS |
| 22-225) | AQKLCEDVKIQYTDTK |
| 43 | MVMRPLWSLLLWEALLPITVTGAQVLSKVGGSVLLVAARPPGFQV |
| (human | REAIWRSLWPSEELLATFFRGSLETLYHSRFLGRAQLHSNLSLELGP |
| SLAMF8, from | LESGDSGNFSVLMVDTRGQPWTQTLQLKVYDAVPRPVVQVFIAVE |
| Uniprot | RDAQPSKTCQVFLSCWAPNISEITYSWRRETTMDFGMEPHSLFTDG |
| Q9P0V8) | QVLSISLGPGDRDVAYSCIVSNPVSWDLATVTPWDSCHHEAAPGKA |
| | SYKDVLLVVVPVSLLLMLVTLFSAWHWCPCSGKKKKDVHADRVG |
| | PETENPLVQDLP |
| 44 | AQVLSKVGGSVLLVAARPPGFQVREAIWRSLWPSEELLATFFRGSL |
| (human | ETLYHSRFLGRAQLHSNLSLELGPLESGDSGNFSVLMVDTRGQPWT |
| SLAMF8, | QTLQLKVYDAVPRPVVQVFIAVERDAQPSKTCQVFLSCWAPNISEIT |
| ectodomain, aa | YSWRRETTMDFGMEPHSLFTDGQVLSISLGPGDRDVAYSCIVSNPV |
| 23-234) | SWDLATVTPWDSCHHEAAPGKASYKDV |
| 85 | MRPQGPAASPQRLRGLLLLLLLQLPAPSSASEIPKGKQKAQLRQREV |
| (Human | VDLYNGMCLQGPAGVPGRDGSPGANGIPGTPGIPGRDGFKGEKGEC |
| Collagen triple | LRESFEESWTPNYKQCSWSSLNYGIDLGKIAECTFTKMRSNSALRVL |
| helix repeat- | FSGSLRLKCRNACCQRWYFTFNGAECSGPLPIEAIIYLDQGSPEMNS |
| containing | TINIHRTSSVEGLCEGIGAGLVDVAIWVGTCSDYPKGDASTGWNSV |
| protein 1 | SRIIIEELPK |
| Isoform 1 Gene | |
| (CTHRC1), | |
| Uniprot | |
| Accession | |
| number | |
| Q96CG8-1) | |
| 86 | MWPPGRSITVKLREKTVSRKLEMNGPSAFQGLICGKYNGMCLQGP |
| (Human | AGVPGRDGSPGANGIPGTPGIPGRDGFKGEKGECLRESFEESWTPNY |
| Collagen triple | KQCSWSSLNYGIDLGKIAECTFTKMRSNSALRVLFSGSLRLKCRNA |
| helix repeat- | CCQRWYFTFNGAECSGPLPIEAIIYLDQGSPEMNSTINIHRTSSVEGL |
| containing | CEGIGAGLVDVAIWVGTCSDYPKGDASTGWNSVSRIIIEELPIYML |
| protein 1 | |
| Isoform 2 Gene | |
| (CTHRC1), | |
| Uniprot | |
| Accession | |
| number | |
| Q96CG8-2) | |
| 87 | MWPPGRSITVKLREKTVSRKLEMNGPSAFQGLICGKYNGMCLQGP |
| (Human | AGVPGRDGSPGANGIPGTPGIPGRDGFKGEKGECLRESFEESWTPNY |
| Collagen triple | KQCSWSSLNYGIDLGKIAECTFTKMRSNSALRVLFSGSLRLKCRNA |
| helix repeat- | CCQRWYFTFNGAECSGPLPIEAIIYLDQGSPEMNSTINIHRTSSVEGL |
| containing | CEGIGAGLVDVAIWVGTCSDYPKGDASTGWNSVSRIIIEELPK |
| protein 1 | |
| Isoform 3 | |
| Gene | |
| (CTHRC1) | |
| Uniprot | |
| Accession | |
| number | |
| Q96CG8-3) | |
| 88 | GAGGTGCAGTTGGTAGAAAGCGGCGGTGGACTCGTGACCCCTGG |
| (AB987 heavy | TGGGTCTCTTCGCTTATCTTGCGCTGCTTCCGGCTTCACTTTTTCT |
| chain | ACGTACAGTATGAATTGGGTTCGACAGGCACCCGGCAAGGGCCT |
| polynucleotide) | GGAATGGGTGTCCAGTATCTCGGGATCCTCAGGACATATTTACTA |
| | CGCTGATAGCGTGAGGGGGCGGTTCACAATCAGCCGTGATAATG |
| | CCAAGAACTCTCTCTATCTACAAATGAACAGTCTGAGAGCAGAG |
| | GACACAGCCGTCTACTACTGTGCCTCAATGCACTACTATTATTAT |
| | CTGGACGTCTGGGGGAAAGGTACTACCGTTACCGTGAGCTCC |
| 89 | CAGGCAGTGGTGACTCAGGAATCCGCTCTTACAACTTCCCCCGG |
| (AB987 light | CGAAACCGTAACATTAACCTGCAGATCAAGTACCGGAACCGTGA |
| chain | CTACATCTAACTATGTCACCTGGGTCCAAGAGAAGCCTGATCAC |
| polynucleotide) | CTGTTCACCGGCTTGATTGGAGCCACAAATAACCGGGCCCCGGG |
| | TGTTCCAGCGAGGTTCAGCGGATCTCTCATCGGTGACAAAGCTG |
| | CCCTCACAATCACCGGGGCTCAGACTGAGGACGAGGCAATATAC |
| | TTTTGTGCCCTGTGGTACAGCAATCATTGGGTTTTTGGTGGGGGC |
| | ACTAAGCTGACGGTGCTA |
| 90 | CAGGTCCAGCCTGTGCAATCAGGAGCCGAAGTTAAGAAGCCCGG |
| (AB988 heavy | GGCTAGCGTTAAAGTGAGTTGTAAAGCTTCCGGTTACACTTTTAC |
| chain | ATCTTATGATATCAACTGGGTGCGCCAGGCGACTGGCCAGGGAC |
| polynucleotide) | TCGAGTGGATGGGCTGGATGAATCCAAATTCCGGTAACACCGGC |
| | TACGCCCAAAAGTTCCAGGGACGGGTGACCATGACCAGGAACAC |
| | ATCCATTTCGACCGCATACATGGAGCTGAGCAGTTTGAGATCCG |
| | AAGATACCGCTGTATATTATTGCGCAATTACAGGCACCACTGCC |
| | GGGACGTTCGACATCTGGGGGCAGGGTACTATGGTGACAGTCTC |
| | TCAGGACATATCAATGATCAGCTCT |
| 91 | GACATCCAGCTCACTCAATCGCCTTCCTTTCTGTCAGCTTCTGTC |
| (AB988 light | GGAGCCAGGGTGACTACCACCTGTCGGGCTAGTCAGGGAATAAG |
| chain | CTCTTACTTGGCATGGTACGAGCAGAAACCAGGGAAGGCACCAA |
| polynucleotide) | AGCTACTGATCTATGCGGCCTCCACTCTGCAGTCAGGTGTGCCTA |
| | GCAGATTCTCTGGCAGCGGGTCTGGCACGGAGTTTACACTTACA |
| | ATTTCCAGCCTCCAACCCGAAGATAGTGCCACATACTATTGCCAG |
| | CAGCACAACTCCTATCCCTGGACCTTCGGCCAGGGTACCAAGGT |
| | TGAAATTAAA |
| 92 | CAGGTGACATTGAGAGAATCCGGCCCCGCACTGGTCAAGCCCAC |
| (AB989 heavy | CCAGACTCTAACATTAACCTGCACTTTTTCTGGGTTCAGTCTGTC |
| chain | GACCAACGTGATGTGCGTCTCATGGATAAGGCAACCTCCAGGTA |
| polynucleotide) | AAGCTCTGGAGTGGCTCGCCCTTATTGACTGGGATGATGACAAA |
| | TATTACAGCACCAGCCTGAAGACGCGGCTCACCATCTCAAAGGA |
| | CACTAGTAAAAACCAGGTGGTTTTGACGATGACCAATATGGATC |
| | CTGTAGACACAGCCACTTACTATTGTGCTCGCACAGGAATCACA |
| | GGGACTCACTACTACTATTACTATATGGATGTGTGGGGCAAGGG |
| | AACAACCGTGACCGTTTCTTCC |
| 93 | GCCATCCAGATGACACAAAGCCCATCCAGTCTGTCTGCCAGCGT |
| (AB989 light | TGGAGACCGGGTGACTATAACCTGTAGAGCATCACAGGGCATCC |
| chain | GCAACGATCTGGGGTGGTATCAGCAGAAGCCAGGTAAAGCACCC |
| polynucleotide) | AAGTTGCTCATTTATGCCGCTTCGTCTCTTCAGTCTGGAGTCCCTT |
| | CCAGGTTTTCCGGCAGTGGTTTCGGCACAGACTTTACTCTAACCA |
| | TTTCAAGCCTGCAACCCGAAGATTTCGCTACCTACTACTGCCTCC |
| | AGGACTATAATTACCCTTGGACGTTCGGACAGGGGACAAAGGTG |
| | GAGATCAAA |
| 94 | GAGGTGCAGCTGGTTGAATCTGGCGGAGGGCTGGTCCAACCCGG |
| (AB990 heavy | AGGCTCCCTGCGCCTCAGTTGCGCTGCATCCGGCTTTACTTTCTC |
| chain | TAACTGTGCTATGAACTGGGTGAGACAGGCCCCTGGTAAAGGTC |
| polynucleotide) | TCGAATGGGTGAGCGTGATTAGCGGCAGCGGCGGAGGTACATAT |
| | TATGCCGATTCCGTAAAAGGGCGATTCACCATCTCACGGGACAA |
| | TTCAAAGAATACCTTGTACCTTCAGATGAACTCTCTAAGGGCAG |
| | AGGACACAGCCGTGTACTATTGCGCCAAGGCTAATTGGAACTAC |
| | GCGTACTTTGATTTCTGGGGACAGGGGACTTTAGTCACCGTTAGT |
| | TCG |
| 95 | GAAATCGTGATGACTCAGTCTCCAGCCACTCTCTCTGTTTCACCC |
| (AB990 light | GGAGAGCGTGCTACACTGTCCTGCAGAGCATCCCAACGAGTGTC |
| chain | CAGCAATCTAGCCTGGTACCAGCAGAAGCCAGGGCAGGCGCCTC |
| polynucleotide) | GCCTGCTTATCTATGGGGCTTCTACACGGGCTACCGGTATTCCCG |
| | CAAGGTTCAGCGGCTCAGGAAGTGGCACCGAGTTTACACTGACC |
| | ATAAACAGTTTGCAGAGCGCCGACTTTGCCGTCTTCTTTTGTCAG |
| | CAGTATGATATCTGGCCTATGTACACGTTCGGCCAAGGTACCAA |
| | ACTCGAAATTAAG |
| 96 | GAGGTGCAGGTTGTCGAATCGGGAGGCGGGCTGGTGCAGCCCGG |
| (AB991 heavy | TGGGAGTCTGAGACTCTCCTGCGCTGCATCAAGGTTCACCTTTAG |
| chain | CAATTACTGGATGTCCTGGGTGCGTCAAGCTCCTGGTAGGGGGC |
| polynucleotide) | TGGAATGGGTCGCCAATATTAAGCAGGATGGTAGCGAGAAGTAC |
| | TATGTCGACTCTGTAAAAGGCCGGTTTACAATCTCCCGAGACAA |
| | CGCCAAAAACAGTCTTTATCTACAGATGAACAGCTTGCGCGCAG |
| | AGGACACTGCCGTGTACTACTGTGCGCGGGAGGGAGGCTATTGC |
| | TCTGATACCAGATGTTTCTTCTATATGGATGTGTGGGGAAAGGGC |
| | ACCACTGTGACAGTTTCTTCA |
| 97 | GACATTCAAATGACTCAATCTCCATCCACTCTATCCGCATCAGTG |
| (AB991 light | GGCGATCGGGTGACCATCACATGTAGAGCTTCCCAGAGCATTAT |
| chain | ATCTTGGTTGGCGTGGTACCAGCAGAAACCTGGCAAGGCTCCCA |
| polynucleotide) | AGCTTCTGATCTATAAGGCCTCATCTCTCGAAGGTGGAGTCCCCA |
| | GCCGCTTTAGCGGGTCCGGGAGTGGTACCGACTTTACGCTGACA |
| | ATCAGTTCGCTGCAGCCTGATGACTTCGCCACCTATTACTGCCAG |
| | CAGTACAACAGCTATTCTAGGACCTTCGGACAGGGCACAACTGT |
| | TGAGATCAAA |
| 98 | GGGGS |
| (Linker) | |
| 99 | GSG |
| (Linker) | |
| 100 | GGGGSGGGGSGGGGS |
| (Linker, ours) | |
| 101 | QVQPVQSGAEVKKPGASVKVSCKASGYTFTSYDINWVRQATGQGL |
| (M23-41BBL) | EWMGWMNPNSGNTGYAQKFQGRVTMTRNTSISTAYMELSSLRSE |
| | DTAVYYCAITGTTAGTFDIWGQGTMVTVSSGGGGSGGGGSGGGGS |
| | DIQLTQSPSFLSASVGARVTTTCRASQGISSYLAWYEQKPGKAPKLL |
| | IYAASTLQSGVPSRFSGSGSGTEFTLTISSLQPEDSATYYCQQHNSYP |
| | WTFGQGTKVEIKGGGGSGGGGSGGGGS |
| | REGPELSPDDPAGLLDLRQGMFAQLVAQNVLLIDGPLSWYSDPGLA |
| | GVSLTGGLSYKEDTKELVVAKAGVYYVFFQLELRRVVAGEGSGSV |
| | SLALHLQPLRSAAGAAALALTVDLPPASSEARNSAFGFQGRLLHLS |
| | AGQRLGVHLHTEARARHAWQLTQGATVLGLFRVTPEIPAGLPSPRS |
| | E |
| 102 | QVQPVQSGAEVKKPGASVKVSCKASGYTFTSYDINWVRQATGQGL |
| (M23 scFv) | EWMGWMNPNSGNTGYAQKFQGRVTMTRNTSISTAYMELSSLRSE |
| | DTAVYYCAITGTTAGTFDIWGQGTMVTVSSGGGGSGGGGSGGGGS |
| | DIQLTQSPSFLSASVGARVTTTCRASQGISSYLAWYEQKPGKAPKLL |
| | IYAASTLQSGVPSRFSGSGSGTEFTLTISSLQPEDSATYYCQQHNSYP |
| | WTFGQGTKVEIK |
| 103 | DIQLTQSPSFLSASVGARVTTTCRASQGISSYLAWYEQKPGKAPKLL |
| (M23-41BBL, | IYAASTLQSGVPSRFSGSGSGTEFTLTISSLQPEDSATYYCQQHNSYP |
| alternate, VH | WTFGQGTKVEIKGGGGSGGGGSGGGGSQVQPVQSGAEVKKPGASV |
| and VL | KVSCKASGYTFTSYDINWVRQATGQGLEWMGWMNPNSGNTGYA |
| reversed) | QKFQGRVTMTRNTSISTAYMELSSLRSEDTAVYYCAITGTTAGTFDI |
| | WGQGTMVTVSSGGGGGGGGSGGGGS |
| | REGPELSPDDPAGLLDLRQGMFAQLVAQNVLLIDGPLSWYSDPGLA |
| | GVSLTGGLSYKEDTKELVVAKAGVYYVFFQLELRRVVAGEGSGSV |
| | SLALHLQPLRSAAGAAALALTVDLPPASSEARNSAFGFQGRLLHLS |
| | AGQRLGVHLHTEARARHAWQLTQGATVLGLFRVTPEIPAGLPSPRS |
| | E |
| 104 | DIQLTQSPSFLSASVGARVTTTCRASQGISSYLAWYEQKPGKAPKLL |
| (M23 alternate | IYAASTLQSGVPSRFSGSGSGTEFTLTISSLQPEDSATYYCQQHNSYP |
| scFv) | WTFGQGTKVEIKGGGGSGGGGSGGGGSQVQPVQSGAEVKKPGASV |
| | KVSCKASGYTFTSYDINWVRQATGQGLEWMGWMNPNSGNTGYA |
| | QKFQGRVTMTRNTSISTAYMELSSLRSEDTAVYYCAITGTTAGTFDI |
| | WGQGTMVTVSS |
| 105 | EVQLVESGGGLVTPGGSLRLSCAASGFTFSTYSMNWVRQAPGKGLE |
| (M5-41BBL) | WVSSISGSSGHIYYADSVRGRFTISRDNAKNSLYLQMNSLRAEDTA |
| | VYYCASMHYYYYLDVWGKGTTVTVSSGGGGSGGGGSGGGGS |
| | QAVVTQESALTTSPGETVTLTCRSSTGTVTTSNYVTWVQEKPDHLF |
| | TGLIGATNNRAPGVPARFSGSLIGDKAALTITGAQTEDEAIYFCALW |
| | YSNHWVFGGGTKLTVLGGGGSGGGGSGGGGS |
| | REGPELSPDDPAGLLDLRQGMFAQLVAQNVLLIDGPLSWYSDPGLA |
| | GVSLTGGLSYKEDTKELVVAKAGVYYVFFQLELRRVVAGEGSGSV |
| | SLALHLQPLRSAAGAAALALTVDLPPASSEARNSAFGFQGRLLHLS |
| | AGQRLGVHLHTEARARHAWQLTQGATVLGLFRVTPEIPAGLPSPRS |
| | E |
| 106 | EVQLVESGGGLVTPGGSLRLSCAASGFTFSTYSMNWVRQAPGKGLE |
| (M5 scFv) | WVSSISGSSGHIYYADSVRGRFTISRDNAKNSLYLQMNSLRAEDTA |
| | VYYCASMHYYYYLDVWGKGTTVTVSSGGGGSGGGGSGGGGS |
| | QAVVTQESALTTSPGETVTLTCRSSTGTVTTSNYVTWVQEKPDHLF |
| | TGLIGATNNRAPGVPARFSGSLIGDKAALTITGAQTEDEAIYFCALW |
| | YSNHWVFGGGTKLTVL |
| 107 | QAVVTQESALTTSPGETVTLTCRSSTGTVTTSNYVTWVQEKPDHLF |
| (M5-41BBL | TGLIGATNNRAPGVPARFSGSLIGDKAALTITGAQTEDEAIYFCALW |
| alternate | YSNHWVFGGGTKLTVL |
| construct) | GGGGSGGGGSGGGGSEVQLVESGGGLVTPGGSLRLSCAASGFTFST |
| | YSMNWVRQAPGKGLEWVSSISGSSGHIYYADSVRGRFTISRDNAKN |
| | SLYLQMNSLRAEDTAVYYCASMHYYYYLDVWGKGTTVTVSSGGG |
| | GSGGGGSGGGGS |
| | REGPELSPDDPAGLLDLRQGMFAQLVAQNVLLIDGPLSWYSDPGLA |
| | GVSLTGGLSYKEDTKELVVAKAGVYYVFFQLELRRVVAGEGSGSV |
| | SLALHLQPLRSAAGAAALALTVDLPPASSEARNSAFGFQGRLLHLS |
| | AGQRLGVHLHTEARARHAWQLTQGATVLGLFRVTPEIPAGLPSPRS |
| | E |
| 108 | QAVVTQESALTTSPGETVTLTCRSSTGTVTTSNYVTWVQEKPDHLF |
| (M5 alternate | TGLIGATNNRAPGVPARFSGSLIGDKAALTITGAQTEDEAIYFCALW |
| scFv) | YSNHWVFGGGTKLTVL |
| | GGGGSGGGGSGGGGSEVQLVESGGGLVTPGGSLRLSCAASGFTFST |
| | YSMNWVRQAPGKGLEWVSSISGSSGHIYYADSVRGRFTISRDNAKN |
| | SLYLQMNSLRAEDTAVYYCASMHYYYYLDVWGKGTTVTVSS |

1. An anti-collagen triple helix repeat containing 1 (anti-CTHRC1) fusion protein, comprising:
(a) a first domain comprising a CTHRC1 binding moiety; and
(b) a second domain comprising a T cell engager; preferably wherein the T cell engager comprises a ligand for a receptor expressed on a T cell.

2. The anti-CTHRC1 fusion protein of claim 1, wherein the T cell engager comprises a ligand for a costimulatory receptor expressed on a T cell, or a functional fragment, variant or mutant thereof; preferably wherein the costimulatory receptor is a member of the tumor necrosis superfamily (TNFSF).

3. The anti-CTHRC1 fusion protein of claim 2, wherein the costimulatory receptor is selected from the group consisting of 4-1BB (CD137), OX40 (CD134), CD40, CD27, CD28, herpesvirus entry mediator (HVEM), ICOS, CD2, and GITR.

4. The anti-CTHRC1 fusion protein of claim 2, wherein:
(a) the costimulatory receptor is 4-1BB (CD137); preferably wherein the ligand comprises an amino acid sequence having at least 80% identity to any one of SEQ ID NOs: 1-10, or a functional fragment thereof;
(b) the costimulatory receptor is OX40 (CD134): wherein the ligand comprises an amino acid sequence having at least 80% identity to any one of SEQ ID NOs: 11-13, or a functional fragment thereof;
(c) the costimulatory receptor is CD40; wherein the ligand comprises an amino acid sequence having at least 80% identity to any one of SEQ ID NOs: 14-16, or a functional fragment thereof;
(d) the costimulatory receptor is GITR; wherein the ligand comprises an amino acid sequence having at least 80% identity to any one of SEQ ID NOs: 17-18, or a functional fragment thereof;
(e) the costimulatory receptor is CD27; wherein the ligand comprises an amino acid sequence having at least 80% identity to any one of SEQ ID NOs: 19-20, or a functional fragment thereof;
(f) the costimulatory receptor is HVEM: wherein the ligand comprises an amino acid sequence having at least 80% identity to any one of SEQ ID NOs: 21-23, or a functional fragment thereof;
(g) the costimulatory receptor is ICOS: wherein the ligand comprises an amino acid sequence having at least 80% identity to any one of SEQ ID NOs: 30-31:
(h) the costimulatory receptor is CD28: wherein the ligand comprises an amino acid sequence having at least 80% identity to any one of SEQ ID NOs: 32-35, or a functional fragment thereof; or
(i) the costimulatory receptor is CD2: wherein the ligand comprises an amino acid sequence having at least 80% identity to any one of SEQ ID NOs: 39-40, or a functional fragment thereof.

5-12. (canceled)

13. The anti-CTHRC1 fusion protein of claim 1, further comprising a linker positioned between the CTHRC1 binding moiety and the ligand; preferably wherein the linker is selected from the group consisting of (G₄S)ₙ or (GSG)ₙ, where n is 1 to 4.

14. The anti-CTHRC1 fusion protein of claim 1, wherein the CTHRC1 binding moiety comprises an anti-CTHRC1 antibody which binds to CTHRC1; preferably wherein the CTHRC1 antibody binds to CTHRC1 with a binding affinity of less than 10 nM.

15. The anti-CTHRC1 fusion protein of claim 14, wherein the anti-CTHRC1 antibody comprises a heavy chain variable region comprising a CDR1 sequence comprising the sequence of SEQ ID NO: 46; a CDR2 sequence comprising the sequence of SEQ ID NO: 51; and a CDR3 sequence comprising the sequence of SEQ ID NO: 56; and a light chain variable region comprising a CDR1 sequence comprising the sequence of SEQ ID NO: 61; a CDR2 sequence comprising the sequence of SEQ ID NO: 66; and a CDR3 sequence comprising the sequence of SEQ ID NO: 71.

16. The anti-CTHRC1 fusion protein of claim 14, wherein the anti-CTHRC1 antibody comprises a heavy chain variable region comprising SEQ ID NO: 77 and a light chain variable region comprising SEQ ID NO: 78.

17-22. (canceled)

23. The anti-CTHRC1 fusion protein of claim 14, wherein the anti-CTHRC1 antibody comprises a heavy chain variable region comprising a CDR1 sequence selected from the group consisting of SEQ ID NOs: 45-49; a CDR2 sequence selected from the group consisting of SEQ ID NOs: 50-54; and a CDR3 sequence selected from the group consisting of SEQ ID NOs: 55-59.

24. The anti-CTHRC1 fusion protein of claim 14, wherein the anti-CTHRC1 antibody comprises a light chain variable region comprising a CDR1 sequence selected from the group consisting of SEQ ID NOs: 60-64; a CDR2 sequence selected from the group consisting of SEQ ID NOs: 65-69; and a CDR3 sequence selected from the group consisting of SEQ ID NOs: 70-74.

25. The anti-CTHRC1 fusion protein of claim 14, wherein the anti-CTHRC1 antibody comprises:
(a) a heavy chain variable region comprising SEQ ID NO: 75 and a light chain variable region comprising SEQ ID NO: 76;
(b) a heavy chain variable region comprising SEQ ID NO: 79 and a light chain variable region comprising SEQ ID NO: 80:
(c) a heavy chain variable region comprising SEQ ID NO: 81 and a light chain variable region comprising SEQ ID NO: 82: or
(d) a heavy chain variable region comprising SEQ ID NO: 83 and a light chain variable region comprising SEQ ID NO: 84.

26-28. (canceled)

29. The anti-CTHRC1 fusion protein of claim 14, wherein the anti-CTHRC1 antibody is a chimeric, humanized, or human antibody.

30. The anti-CTHRC1 fusion protein of claim 14, wherein the anti-CTHRC1 antibody is a monoclonal antibody.

31. The anti-CTHRC1 fusion protein of claim 14, wherein the anti-CTHRC1 antibody is an antibody fragment.

32. The anti-CTHRC1 fusion protein of claim 14, wherein the anti-CTHRC1 antibody comprises a single-chain antibody.

33. The anti-CTHRC1 fusion protein of claim 14, wherein the anti-CTHRC1 antibody is a heavy-chain only antibody (single domain antibody).

34. A method for activating T cells in a tumor microenvironment, comprising contacting a tumor with the anti-CTHRC1 fusion protein of claim 1.

35. A method of inhibiting the growth of a cell displaying a CTHRC1 tumor epitope, comprising contacting said cell with the anti-CTHRC1 fusion protein of claim 1 to the subject.

36. A method of treating a subject having cancer, comprising administering the anti-CTHRC1 fusion protein of claim 1.

37. The method of claim 36, wherein the cancer is selected from the group consisting of breast cancer, lung cancer, colon cancer, ovarian cancer, melanoma cancer, bladder cancer, renal cancer, kidney cancer, liver cancer, head and neck cancer, colorectal cancer, pancreatic cancer, gastric carcinoma cancer, esophageal cancer, mesothelioma, prostate cancer, leukemia, lymphomas, and myelomas.

38. The method of claim 36, further comprising administering an allogenic or autologous T cell therapy in combination with the anti-CTHRC1 fusion protein of claim 1.

39. A pharmaceutical composition comprising the anti-CTHRC1 fusion protein of claim 1 and a pharmaceutically acceptable carrier.

40. (canceled)
